(19) United States
(12) Patent Application Publication
(10) Pub. No.: US 2026/0242432 A1
(43) Pub. Date: Aug. 20, 2026

(54) MODIFIED FC POLYPEPTIDES WITH ENHANCED SIALYLATION

(71) Applicant: Nuvig Therapeutics, Inc., Menlo Park, CA (US)

(72) Inventors: Pamela B. Conley, Palo Alto, CA (US); Greg P. Coffey, San Carlos, CA (US); Mark J. Karbarz, Burlingame, CA (US)

(21) Appl. No.: 19/110,756

(22) PCT Filed: Sep. 13, 2023
(86) PCT No.: PCT/US2023/074097

§ 371 Date: Mar. 11, 2025

Related U.S. Application Data
(60) Provisional application No. 63/375,490, filed on Sep. 13, 2022.
(60) Provisional application No. 63/512,013, filed on Jul. 5, 2023.

(51) Int. Cl. C07K 14/47; A61K 38/00; A61K 45/06; A61P 37/06; C12N 9/10; C12N 15/85

(52) U.S. Cl. CPC C07K 14/47; A61K 45/06; A61P 37/06; C12N 9/1081; C12N 15/85; C12Y 204/99001; A61K 38/00

(57) ABSTRACT

The present disclosure relates to compositions containing highly sialylated immunoglobulin Fc polypeptides having an amino acid substitution from phenylalanine to an aliphatic amino acid residue, such as alanine, at amino acid residue 241 of the Fc heavy chain. Further disclosed are methods of manufacturing the highly sialylated Fc polypeptides in a recombinant expression system. Also disclosed are methods of using the disclosed compositions for the treatment of an inflammatory disease or disorder.

### CROSS-REFERENCE TO RELATED APPLICATIONS

This application claims the benefit of and priority to U.S. Provisional Patent Application Nos. 63/375,490 (filed on Sep. 13, 2022) and 63/512,013 (filed on Jul. 5, 2023), the disclosures of each of which are incorporated by reference herein in their entirety for all purposes.

### SEQUENCE LISTING

The present specification makes reference to a Sequence listing which has been submitted electronically in XML format and is hereby incorporated by reference in its entirety. Said XML file, created on Sep. 12, 2023, is named NVG_003WO_ST26.xml and is 8 kb in size.

### BACKGROUND

Inflammatory disorders, including autoimmune diseases, are disorders involving abnormal activation and subsequent migration of white blood cells to affected areas of the body. These conditions encompass a wide range of ailments that affect the lives of millions of people throughout the world. Although various treatments are presently available, many possess significant side effects or are insufficiently effective in alleviating symptoms.

Immunoglobulin G (IgG) has long been appreciated to mediate both pro- and anti-inflammatory activities through interactions mediated by its fragment crystallizable (Fc) region. While Fc-FcγR interactions are responsible for the pro-inflammatory properties of immune complexes and cytotoxic antibodies, intravenous gamma globulin (IVIG) and its constituent Fc fragments are anti-inflammatory and are widely used to suppress inflammation in disease states.

Sialylation of recombinant therapeutic glycoproteins, such as Fc polypeptides, is generally performed in mammalian cell lines capable of reproducing mammalian or mammalian-like glycosylation profiles.

### SUMMARY

The present disclosure provides compositions comprising Fc polypeptides having a high degree of sialylation at their N-glycan and methods of producing and using the same.

Disclosed herein, in certain embodiments, are populations of modified Fc polypeptides, each modified Fc polypeptide having (i) an amino acid sequence at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) identical to the sequence of SEQ ID NO: 2 and (ii) an aliphatic amino acid residue (e.g., alanine, glycine, isoleucine, leucine, proline, valine, and methionine) at position 241 (numbered according to Kabat; corresponding to amino acid residue 32 of SEQ ID NO: 2), comprising: at least 60% (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the modified Fc polypeptides having a sialic acid (SA) moiety attached to an N-glycan of the Fc polypeptide via an α(2,6) linkage. In some embodiments, the aliphatic amino acid at position 241 is an alanine (Ala; F241A). In some embodiments, the aliphatic amino acid at position 241 is a glycine (Gly; F241G). In some embodiments, the aliphatic amino acid at position 241 is an isoleucine (Ile; F241I). In some embodiments, the aliphatic amino acid at position 241 is a leucine (Leu; F241L). In some embodiments, the aliphatic amino acid at position 241 is a proline (Pro; F241P). In some embodiments, the aliphatic amino acid at position 241 is a valine (Val; F241V). In some embodiments, the aliphatic amino acid at position 241 is a methionine (Met; F241M). In some embodiments, the N-glycan is attached to the asparagine (Asn) at amino acid residue 297 of the polypeptide (Asn297; numbered according to Kabat; corresponding to amino acid residue 88 of SEQ ID NO: 2). In some embodiments, at least 70% (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the Fc polypeptides comprise the SA moiety attached to the N-glycan of the Fc polypeptides via the α(2,6) linkage. In some embodiments, at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) of the Fc polypeptides comprise the SA moiety attached to the N-glycan of the Fc polypeptides via the α(2,6) linkage. In some embodiments, at least 90% (e.g., at least 90%, 91%, 92%, 93%, 94%, 95%, 96%, 97%, 98%, 99%, or 100%) of the Fc polypeptides comprise the SA moiety attached to the N-glycan of the Fc polypeptides via the α(2,6) linkage. In some embodiments, the N-glycan of the Fc polypeptides is mono-sialylated or di-sialylated. In some embodiments, at least 30% (e.g., at least 30%, 40%, 50%, 60%, 70%, 80%, 90%, 95%, 99%, or more) of the Fc polypeptides comprise mono-sialylated N-glycans comprising a SA moiety attached via the α(2,6) linkage. In some embodiments, at least 30% (e.g., at least 30%, 40%, 50%, 60%, 70%, 80%, 90%, 95%, 99%, or more) of the Fc polypeptides comprise di-sialylated N-glycans comprising two SA moieties attached via the α(2,6) linkage. In some embodiments, about 90% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) of the Fc polypeptides comprise di-sialylated N-glycans comprising two SA moieties attached via the α(2,6) linkage. In some embodiments, at least about 60% (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the Fc polypeptides comprise a galactose moiety. In some embodiments, at least about 70% (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the Fc polypeptides comprise a galactose moiety. In some embodiments, at least about 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) of the Fc polypeptides comprise a galactose moiety. In some embodiments, at least about 90% (e.g., at least 90%, 91%, 92%, 93%, 94%, 95%, 96%, 97%, 98%, 99%, or 100%) of the Fc polypeptides comprise a galactose moiety. In some embodiments, about 100% of the Fc polypeptides comprise a galactose moiety. In some embodiments, the galactose moiety is attached to an α(1,3) arm and/or α(1,6) arm of the N-glycan. In some embodiments, the galactose moiety is a branched galactose moiety.

Disclosed herein, in certain embodiments, are populations of modified Fc polypeptides, each modified Fc polypeptide having (i) an amino acid sequence at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) identical to the sequence of SEQ ID NO: 2 and (ii) an aliphatic amino acid residue (e.g., alanine, glycine, isoleucine, leucine, proline, valine, and methionine) at position 241 (numbered according to Kabat; corresponding to amino acid residue 32 of SEQ ID NO: 2), comprising: about 40% (e.g., 30%, 31%, 32%, 33%, 34%, 35%, 36%, 37%, 38%, 39% 40%, 41%, 42%, 43%, 44%, 45%, 46%, 47%, 48%, 49%, and 50%) of the modified Fc polypeptides having a SA moiety attached to the N-glycan of the Fc polypeptide via an α(2,3) linkage. In some embodiments, the aliphatic amino acid at position 241 is an alanine (Ala; F241A). In some embodiments, the aliphatic amino acid at position 241 is a glycine (Gly; F241G). In some embodiments, the aliphatic amino acid at position 241 is an isoleucine (Ile; F241I). In some embodiments, the aliphatic amino acid at position 241 is a leucine (Leu; F241L). In some embodiments, the aliphatic amino acid at position 241 is a proline (Pro; F241P). In some embodiments, the aliphatic amino acid at position 241 is a valine (Val; F241V). In some embodiments, the aliphatic amino acid at position 241 is a methionine (Met; F241M). In some embodiments, the N-glycan is attached to the asparagine (Asn) at amino acid residue 297 of the polypeptide (Asn297; numbered according to Kabat; corresponding to amino acid residue 88 of SEQ ID NO: 2). In some embodiments, the Fc polypeptides are IgG1. In some embodiments, the Fc polypeptides are IgG3.

Disclosed herein, in certain embodiments, are pharmaceutical compositions, comprising: (a) a population of modified Fc polypeptides of any of the foregoing embodiments, and (b) a pharmaceutically acceptable carrier, diluent, or excipient. In some embodiments, the pharmaceutically acceptable carrier, diluent, or excipient is selected from the group consisting of: a stabilizer, buffer, surfactant, filler, solvent, tonicity or osmolarity adjusting agent, antioxidant, adjuvant, and antimicrobial agent.

Disclosed herein, in certain embodiments, are methods of treating an inflammatory disease or condition in a subject (e.g., a human) in need thereof, comprising: administering to the subject a therapeutically effective amount of a population of modified Fc polypeptides of any of the foregoing embodiments. In some embodiments, the inflammatory disease or condition is an autoimmune disease or condition. In some embodiments, the inflammatory disease or condition is arthritis. In some embodiments, the inflammatory disease or condition is immune thrombocytopenia (ITP). In some embodiments, the population or the pharmaceutical composition has a half-life of at least 3.5 days (e.g., at least 3.5, 4, 4.5, 5, 6, 7, 8, 9, 10, or more days) following administration of the population or the pharmaceutical composition to the subject. In some embodiments, the population or the pharmaceutical composition has a half-life of at least 4 days (e.g., at least 4, 4.5, 5, 6, 7, 8, 9, 10, or more days) following administration of the population or the pharmaceutical composition to the subject. In some embodiments, the population or the pharmaceutical composition is cleared from the circulation of the subject at a rate of no greater than 45 mL/day/kg (e.g., no greater than 45, 40, 35, 30, 25, 20 mL/day/kg, or less). In some embodiments, the population or the pharmaceutical composition is cleared from the circulation of the subject at a rate of no greater than 40 mL/day/kg (e.g., no greater than 40, 35, 30, 25, 20 mL/day/kg, or less). In some embodiments, the population or the pharmaceutical composition is cleared from the circulation of the subject at a rate of no greater than 35 mL/day/kg (e.g., no greater than 35, 30, 25, 20 mL/day/kg, or less). In some embodiments, the population or the pharmaceutical composition is cleared from the circulation of the subject at a rate of no greater than 30 mL/day/kg (e.g., no greater than 30, 25, 20 mL/day/kg, or less). In some embodiments, the population or the pharmaceutical composition is cleared from the circulation of the subject at a rate of no greater than 25 mL/day/kg (e.g., no greater than 25, 25, 20 mL/day/kg, or less). In some embodiments, the concentration of the Fc polypeptide over time (AUC) is at least 480 day×mg/mL between 1 and 35 days (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days) following administration of the population or the pharmaceutical composition to the subject. In some embodiments, the AUC is at least 500 day×mg/mL, at least 550 day×mg/mL, at least 600 day×mg/mL, at least 650 day×mg/mL, at least 700 day×mg/mL, at least 750 day×mg/mL, at least 800 day×mg/mL, or at least 850 day×mg/mL between 1 and 35 days (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days) following administration of the population or the pharmaceutical composition to the subject. In some embodiments, the method further comprises administration of an additional therapeutic agent to the subject. In some embodiments, the additional therapeutic agent is administered to the subject prior to, concurrently with, or subsequent to administration of the composition or the pharmaceutical composition. In some embodiments, the additional therapeutic agent is a second modified Fc polypeptide comprising one or more amino acid substitutions selected from the group consisting of M252Y, S254T, T256E, H433K, and N434F. In some embodiments, the second modified Fc polypeptide comprises amino acid substitutions M252Y, S254T, T256E, H433K, and N434F. In some embodiments, the second modified Fc polypeptide has an amino acid sequence set forth as SEQ ID NO: 5. In some embodiments, the second modified Fc polypeptide is administered at a dose of 1 mg/kg to 20 mg/kg (e.g., 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg). In some embodiments, the second modified Fc polypeptide is administered at a dose of 10 mg/kg. In some embodiments, the second modified Fc polypeptide is administered once weekly for four weeks. In some embodiments, the additional therapeutic agent is selected from the group consisting of an anti-inflammatory agent, an immune-suppressive agent, an analgesic, a disease-modifying antirheumatic drug (DMARD), a counterirritant, a platelet-boosting drug, a thrombopoietin receptor (TPOR) agonist, physical therapy, and surgery. In some embodiments, the anti-inflammatory agent is selected from the group consisting of non-steroidal anti-inflammatory drug (NSAID), corticosteroid, anti-inflammatory antibody or an antigen-binding fragment thereof, anti-inflammatory cytokine, kinase inhibitor, and intravenous immunoglobulin (IVIG).

Disclosed herein, in certain embodiments, are nucleic acid expression vectors, comprising: (a) a first expression cassette, comprising: a first mammalian promoter operably linked to a polynucleotide encoding a modified Fc polypeptide having: (i) an amino acid sequence at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) identical to the sequence of SEQ ID NO: 2 and (ii) an aliphatic amino acid residue (e.g., alanine, glycine, isoleucine, leucine, proline, valine, and methionine) at position 241 (numbered according to Kabat; corresponding to amino acid residue 32 of SEQ ID NO: 2); and (b) a second expression cassette, comprising: a second mammalian promoter operably linked to a polynucleotide encoding a beta-galactoside alpha-2,6-sialyltransferase 1 (ST6GAL1) enzyme. In some embodiments, the aliphatic amino acid at position 241 is an Ala (F241A). In some embodiments, the aliphatic amino acid at position 241 is an alanine (Ala; F241A). In some embodiments, the aliphatic amino acid at position 241 is a glycine (Gly; F241G). In some embodiments, the aliphatic amino acid at position 241 is an isoleucine (Ile; F241I). In some embodiments, the aliphatic amino acid at position 241 is a leucine (Leu; F241L). In some embodiments, the aliphatic amino acid at position 241 is a proline (Pro; F241P). In some embodiments, the aliphatic amino acid at position 241 is a valine (Val; F241V). In some embodiments, the aliphatic amino acid at position 241 is a methionine (Met; F241M). In some embodiments, the second expression cassette further comprises a polynucleotide encoding a beta-1,4-galactosyltransferase 1 (B4GALT1) enzyme. In some embodiments, the polynucleotide encoding the B4GALT1 enzyme is operatively linked to the second promoter. In some embodiments, the second expression cassette further comprises an internal ribosome entry site (IRES) sequence positioned between the polynucleotide encoding the ST6GAL1 enzyme and the polynucleotide encoding the B4GALT1 enzyme. In some embodiments, the first promoter and the second promoter are each independently selected from the group consisting of a murine cytomegalovirus (CMV) promoter, elongation factor 1α (EF1α) promoter, eukaryotic elongation factor 2 (EEF2) promoter, glyceraldehyde 3-phosphate dehydrogenase (GAPDH) promoter, phosphoglycerate kinase (PGK) promoter, actin promoter, and ubiquitin promoter.

Disclosed herein, in certain embodiments, are mammalian host cells, comprising: an expression vector of the foregoing embodiments. In some embodiments, the mammalian host cell is a Chinese hamster ovary (CHO) cell. In some embodiments, the mammalian host cell is a human embryonic kidney 293 (HEK293) cell.

Disclosed herein, in certain embodiments, are methods of manufacturing a population of modified Fc polypeptides, each modified Fc polypeptide having: (i) an amino acid sequence having at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) identity to SEQ ID NO: 2, and (ii) an aliphatic amino acid residue (e.g., alanine, glycine, isoleucine, leucine, proline, valine, and methionine) at position 241 (numbered according to Kabat; corresponding to amino acid residue 32 of SEQ ID NO: 2), the method comprising: (a) culturing a mammalian host cell of the foregoing embodiments under conditions and for a time that induces expression of the first expression cassette and the second expression cassette, thus producing the population of modified Fc polypeptides; and (b) purifying the population of modified Fc polypeptides. In some embodiments, the N-glycan is attached to the asparagine (Asn) at amino acid residue 297 of the polypeptide (Asn297; numbered according to Kabat; corresponding to amino acid residue 88 of SEQ ID NO: 2). In some embodiments, at least 70% (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the Fc polypeptides comprise the SA moiety attached to the N-glycan of the Fc polypeptides via the α(2,6) linkage. In some embodiments, at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) of the Fc polypeptides comprise the SA moiety attached to the N-glycan of the Fc polypeptides via the α(2,6) linkage. In some embodiments, at least 90% (e.g., at least 90%, 91%, 92%, 93%, 94%, 95%, 96%, 97%, 98%, 99%, or 100%) of the Fc polypeptides comprise the SA moiety attached to the N-glycan of the Fc polypeptides via the α(2,6) linkage. In some embodiments, the first promoter and the second promoter are each independently selected from the group consisting of a murine CMV promoter, EF1α promoter, EEF2 promoter, GAPDH promoter, PGK promoter, actin promoter, and ubiquitin promoter. In some embodiments, the first expression cassette and/or the second expression cassette each independently comprise one or more regulatory sequences selected from the group consisting of a 5′ untranslated region (UTR), 3′ UTR, enhancer, insulator, intron, RNA export element, polyadenylation signal, internal ribosome entry site (IRES), and transcription terminator. In some embodiments, the N-glycan of the Fc polypeptides is mono-sialylated or di-sialylated. In some embodiments, at least 30% (e.g., at least 30%, 40%, 50%, 60%, 70%, 80%, 90%, 95%, 99%, or more) of the Fc polypeptides comprise mono-sialylated N-glycans comprising a SA moiety attached via the α(2,6) linkage. In some embodiments, at least 30% (e.g., at least 30%, 40%, 50%, 60%, 70%, 80%, 90%, 95%, 99%, or more) of the Fc polypeptides comprise di-sialylated N-glycans comprising two SA moieties attached via the α(2,6) linkage. In some embodiments, about 90% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) of the Fc polypeptides comprise di-sialylated N-glycans comprising two SA moieties attached via the α(2,6) linkage. In some embodiments, at least about 60% (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the Fc polypeptides comprise a galactose moiety. In some embodiments, at least about 70% (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the Fc polypeptides comprise a galactose moiety. In some embodiments, at least about 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) of the Fc polypeptides comprise a galactose moiety. In some embodiments, at least about 90% (e.g., at least 90%, 91%, 92%, 93%, 94%, 95%, 96%, 97%, 98%, 99%, or 100%) of the Fc polypeptides comprise a galactose moiety. In some embodiments, about 100% of the Fc polypeptides comprise a galactose moiety. In some embodiments, the galactose moiety is attached to an α(1,3) arm and/or α(1,6) arm of the N-glycan. In some embodiments, the galactose moiety is a branched galactose moiety. In some embodiments, the mammalian host cell comprises ST6GAL1 and B4GALT1 at a ratio of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20 (mol:mol). In some embodiments, the mammalian host cell is in a population of mammalian host cells in which at least 50%, at least 60%, at least 70%, at least 80%, at least 90%, or more of the mammalian host cells remain viable between 10 days and 20 days (e.g., 10, 11, 12, 13, 14, 15, 16, 17, 18, 19, or 20 days) following the start of step (a). In some embodiments, the method further comprises contacting the mammalian host cell with an additive that enhances sialylation of the Fc polypeptides. In some embodiments, the additive is selected from the group consisting of uridine, manganese, copper, dexamethasone, hydrocortisone, N-acetyl mannosamine, tetraacetylated ManNAc, N-azidoacetyl D-mannosamine, 1,3,4-O-Bu₃ManNAc, α(2,3)-Dehydro-2-deoxy-N-acetylneuraminic acid (DANA), siastatin B, fetuin, and glycerol. In some embodiments, glycosylation of the N-glycan is determined using HPLC, MS, or a combination thereof. In some embodiments, the HPLC is hydrophilic interaction liquid chromatography (HILIC).

Disclosed herein, in certain embodiments, are population or pharmaceutical compositions (as disclosed here) for use as a medicament.

Disclosed herein, in certain embodiments, are population or pharmaceutical compositions (as disclosed here) for in the treatment of an inflammatory disease or condition, e.g., an autoimmune disease or condition, in a subject in need thereof. For example, the inflammatory disease or condition may be arthritis or immune thrombocytopenia (ITP).

In some embodiments, the population or the pharmaceutical composition has a half-life of at least 3.5 or at least 4 days following administration of the population or the pharmaceutical composition to a subject.

In some embodiments, the population or the pharmaceutical composition, following administration to a subject, is cleared from the circulation of the subject at a rate of no greater than 45 mL/day/kg, no greater than 40 mL/day/kg, no greater than 35 mL/day/kg, no greater than 30 mL/day/kg, or no greater than 25 mL/day/kg.

In some embodiments, the concentration of the Fc polypeptide over time (AUC) is at least 480 day×mg/mL between 1 and 35 days following administration of the population or the pharmaceutical composition to a subject, e.g., at least 500 day×mg/mL, at least 550 day×mg/mL, at least 600 day×mg/mL, at least 650 day×mg/mL, at least 700 day×mg/mL, at least 750 day×mg/mL, at least 800 day×mg/mL, or at least 850 day×mg/mL between 1 and 35 days following administration of the population or the pharmaceutical composition to a subject.

In some embodiments, an additional therapeutic agent is administered to the subject. In some embodiments, the additional therapeutic agent is administered to the subject prior to, concurrently with, or subsequent to administration of the population or the pharmaceutical composition.

In some embodiments, the additional therapeutic agent is a second modified Fc polypeptide comprising one or more amino acid substitutions selected from the group consisting of M252Y, S254T, T256E, H433K, and N434F. In some embodiments, the second modified Fc polypeptide comprises amino acid substitutions M252Y, S254T, T256E, H433K, and N434F. In some embodiments, the second modified Fc polypeptide has an amino acid sequence set forth as SEQ ID NO: 5.

In some embodiments, the second modified Fc polypeptide is administered to a subject at a dose of 1 mg/kg to 20 mg/kg, e.g., at a dose of 10 mg/kg. In some embodiments, the second modified Fc polypeptide is administered to a subject once weekly for four weeks.

In some embodiments, the additional therapeutic agent is selected from the group consisting of an anti-inflammatory agent, an immune-suppressive agent, an analgesic, a disease-modifying antirheumatic drug (DMARD), a counterirritant, a platelet-boosting drug, a thrombopoietin receptor (TPOR) agonist, physical therapy, and surgery.

In some embodiments, the anti-inflammatory agent is selected from the group consisting of non-steroidal anti-inflammatory drug (NSAID), corticosteroid, anti-inflammatory antibody or an antigen-binding fragment thereof, anti-inflammatory cytokine, kinase inhibitor, and intravenous immunoglobulin (IVIG).

### Definitions

Unless defined otherwise, all technical and scientific terms used herein have the same meaning as is commonly understood by one of skill in the art to which the claimed subject matter belongs. Generally, nomenclatures utilized in connection with and techniques of immunology, oncology, cell and tissue culture, molecular biology, and protein chemistry described herein are those well-known and commonly used in the art. It is to be understood that the foregoing general description and the following detailed description are exemplary and explanatory only and are not restrictive of any subject matter claimed. The section headings used herein are for organizational purposes only and are not to be construed as limiting the subject matter described.

As used herein, singular forms “a,” “and,” and “the” include plural referents unless the context clearly indicates otherwise. Thus, e.g., reference to “a polypeptide” includes a plurality of polypeptides.

As used herein, all numerical values or numerical ranges include whole integers within or encompassing such ranges and fractions of the values or the integers within or encompassing ranges unless the context clearly indicates otherwise. Thus, e.g., reference to a range of 90-100%, includes 91%, 92%, 93%, 94%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20 fold, etc., as well as 1.1, 1.2, 1.3, 1.4, 1.5 fold, etc., 2.1, 2.2, 2.3, 2.4, 2.5 fold, etc., and so forth.

“About” a number, as used herein, refers to range including the number and ranging from 10% below that number to 10% above that number. “About” a range refers to 10% below the lower limit of the range, spanning to 10% above the upper limit of the range.

As used herein, “administration” refers to providing or giving a subject a therapeutic agent (e.g., a modified Fc polypeptide of the disclosure or a composition containing the same) by any effective route. Exemplary routes of administration are described in the sections that follow.

The term “effective amount” as used herein, refers to that amount of Fc polypeptides or compositions of the disclosure that is sufficient to induce a disclosed effect, e.g., to effect treatment, prognosis, or diagnosis of a disease (e.g., inflammatory disease or disorder), as described herein, when administered to a subject. Therapeutically effective amounts of the compositions provided herein, when used alone or in combination, will vary depending upon the relative activity of the disclosed compositions and combinations (e.g., in treating, reducing, or ameliorating a disease or disorder described herein) and depending upon the subject and disease condition being treated, the weight and age of the subject, the severity of the disease condition, the manner of administration, and the like.

As used herein, the terms “Fc polypeptide,” “Fc peptide,” “Fc fragment,” “Fc region,” and “Fc domain” are interchangeably used to define a C-terminal region of an immunoglobulin heavy chain. The “Fc polypeptide” is a native sequence Fc region or a variant Fc region, in some embodiments. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof.

An “isolated” polypeptide or protein or a population thereof refers to a polypeptide or protein or a population thereof that has been separated from other proteins, lipids, and nucleic acids with which it is naturally associated. The polypeptide/protein or a population thereof constitutes at least 10% (i.e., any percentage between 10% and 100%, e.g., 20%, 30%, 40%, 50%, 60%, 70%, 80%, 85%, 90%, 95%, and 99%) by dry weight of the purified preparation, in some embodiments. Purity can be measured by any appropriate standard method, for example, by column chromatography, polyacrylamide gel electrophoresis, high-performance liquid chromatography (HPLC), size exclusion chromatography (SEC), or mass spectrometry (MS) analysis. An isolated polypeptide/protein or a population thereof described herein produced by recombinant DNA techniques or by chemical methods, in some embodiments. The isolated polypeptide includes SEQ ID NO: 2, in some embodiments.

A “native” or “parental” Fc region comprises an amino acid sequence identical to the amino acid sequence of an Fc region found in nature. A “variant” or “modified” Fc region includes an amino acid sequence which differs from that of a native sequence Fc region by virtue of at least one amino acid modification, such as an amino acid substitution (e.g., F241A). In some embodiments, a modified Fc region has at least one amino acid substitution compared to a native sequence Fc region or to the Fc region of a parent polypeptide, e.g., from about one to about ten amino acid substitutions. The modified Fc region described herein will, in some embodiments, possess at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) sequence identity with the native sequence Fc region and/or with an Fc region of a parent polypeptide.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Appropriate parameters for aligning sequences, including any algorithms needed to achieve maximal alignment over the full length of the sequences being compared, are determined by any suitable means. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutically acceptable” refers to those compounds, materials, compositions, and/or dosage forms, which are suitable for contact with the tissues of a subject, such as a mammal (e.g., a human) without excessive toxicity, irritation, allergic response, and other problem complications commensurate with a reasonable benefit/risk ratio.

The term “polypeptide” refers to a chain of amino acids. The polypeptides are not limited to a specific length of the product. Peptides, oligopeptides, and proteins are included within the definition of polypeptide, and such terms are used interchangeably herein unless specifically indicated otherwise. This term also encompasses chains of peptides with post-expression modifications, e.g., glycosylation, acetylation, phosphorylation, and the like, as well as other modifications known in the art, both naturally occurring and non-naturally occurring. In some embodiments, a polypeptide is an entire protein, or a fragment thereof.

The terms “recipient,” “individual,” “subject,” “host,” and “patient,” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys, etc. In some embodiments, the mammal is a human. None of the terms require the supervision of a medical professional.

The term “therapeutically effective amount” generally refers to an amount of a disclosed composition effective to “treat” a disease or disorder in a subject or mammal. In some embodiments, a composition described herein is administered to a subject in an amount that is effective for producing some desired therapeutic effect by inhibiting a disease or disorder as described herein at a reasonable benefit/risk ratio applicable to any medical treatment. A therapeutically effective amount is an amount that achieves at least partially a desired therapeutic or prophylactic effect in an organ or tissue. The amount of a therapeutic agent necessary to bring about prevention and/or therapeutic treatment of a disease or disorder is not fixed per se. In some embodiments, the amount of the therapeutic agent administered varies with the type and extensiveness of the disease, and the size of the mammal suffering from the disease or disorder. When used in conjunction with therapeutic methods involving administration of a therapeutic agent after the subject presents symptoms of a disease or disorder, the term “therapeutically effective” means that, after treatment, one or more signs or symptoms of the disease or disorder is ameliorated or eliminated.

An effective response of the present disclosure is achieved when the subject experiences partial or total alleviation or reduction of signs or symptoms of illness and, in the case of the treatment of a disease (e.g., an inflammatory disease or disorder), specifically includes, without limitation, amelioration of symptoms, prolongation of progression, cure, remission, prolongation of survival, or other objective responses. In some embodiments, the expected progression-free survival times are measured in months to years, depending on prognostic factors including the number of relapses, stage of disease, and other factors. Prolonging survival includes without limitation times of at least 1 month (mo.), about at least 2 mos., about at least 3 mos., about at least 4 mos., about at least 6 mos., about at least 1 year, about at least 2 years, about at least 3 years, etc. Overall survival is also measured, e.g., in months to years. Alternatively, an effective response, in some embodiments, is that a subject's symptoms remain static. Further indications of treatment of indications are described in more detail below.

In some embodiments, administration of a therapeutic agent in a prophylactic method occurs prior to the manifestation of symptoms of an undesired disease or disorder, such that the disease or disorder is prevented or, alternatively, delayed in its progression. Thus, when used in conjunction with prophylactic methods, the term “therapeutically effective” means that, after treatment, a smaller number of subjects (on average) develop the undesired disease or disorder or progress in severity of symptoms.

As used herein, the terms “treatment,” “treating,” and the like, in some cases, refer to administering an agent, or carrying out a procedure, for the purposes of obtaining an effect. The effect may be prophylactic in terms of completely or partially preventing a disease or symptom thereof and/or is therapeutic in terms of effecting a partial or complete cure for a disease and/or symptoms of the disease. “Treatment,” as used herein, includes treatment of a disease or disorder (e.g., an inflammatory disease or disorder) in a mammal, particularly in a human, and includes: (a) preventing the disease or a symptom of a disease from occurring in a subject which is predisposed to the disease but has not yet been diagnosed as having it (e.g., including diseases associated with or caused by a primary disease; (b) inhibiting the disease, i.e., arresting its development; and (c) relieving the disease, i.e., causing regression of the disease. The term treating includes any indicia of success in the treatment, amelioration, or prevention of a disease or disorder, including any objective or subjective parameter such as abatement, remission, diminishing of symptoms or making the disease condition more tolerable to the patient, slowing in the rate of degeneration or decline, or making the final point of degeneration less debilitating. The treatment or amelioration of symptoms is based on one or more objective or subjective parameters, including the results of an examination by a physician. Accordingly, the term “treating” includes the administration of the agents or compositions of the present disclosure to prevent or delay, to alleviate, or to arrest or inhibit development of the symptoms or conditions associated with diseases. The term “therapeutic effect” refers to the reduction, elimination, or prevention of the disease, symptoms of the disease, or side effects of the disease in the subject. A subject is “treated” for a disease or disorder if, after receiving a therapeutic amount of a therapeutic agent or composition of the present disclosure, the patient shows observable and/or measurable change in a parameter or symptom of the disease or disorder.

As used herein, the term “vector” includes a nucleic acid vector, e.g., a DNA vector, such as a plasmid, an RNA vector, virus, or other suitable replicon (e.g., viral vector). A variety of vectors have been developed for the delivery of polynucleotides encoding exogenous proteins into a prokaryotic or eukaryotic cell. Expression vectors suitable for use with the compositions and methods described herein contain an expression cassette comprising a polynucleotide sequence as well as, e.g., additional sequence elements used for the expression of proteins and, optionally, the integration of these polynucleotide sequences into the genome of a host cell. Certain vectors that can be used for the expression one or more (e.g., 1, 2, 3, or more) recombinant polypeptides, as described herein, include plasmids that contain regulatory sequences, such as promoter and enhancer elements which direct gene transcription. Other useful vectors for expression of disclosed polypeptides contain polynucleotide sequences that enhance the rate of translation of these genes or improve the stability or nuclear export of the mRNA that results from gene transcription. These sequence elements may include, e.g., 5′ and 3′ untranslated regions (UTRs), an internal ribosomal entry site (IRES), and a polyadenylation signal site in order to direct efficient transcription of the transgene carried on the expression vector. The expression vectors suitable for use with the compositions and methods described herein may also contain a polynucleotide encoding a marker for selection of cells that contain such a vector. An example of a suitable selection marker is a glutamine synthetase (GS) gene. Additional examples of a suitable marker are genes that encode resistance to antibiotics, such as ampicillin, chloramphenicol, kanamycin, nourseothricin, zeocin, nourseothricin, carbenicillin, tetracycline, streptomycin, and spectinomycin.

### BRIEF DESCRIPTION OF THE DRAWINGS

FIG. 1 shows illustrations and nomenclature of representative bi-antennary N-glycan structures found on Asparagine 297 (Asn297) of the heavy chain of an Fc polypeptide of the disclosure. Exemplified glycoforms attach to a heavy chain constant region 2 (CH2) domain of the Fc polypeptide via a covalent bond at the terminal end on the bottom end of each glycan structure. All subsequent descriptions of N-glycans are provided as linear or branched sequences of carbohydrates that start at Asn297 (e.g., Asn297→Carbohydrate 1→Carbohydrate 2, etc.). Carbohydrates closest to Asn297 are considered proximal, those further from Asn297 are considered distal, and those furthest from Asn297 are considered terminal. The G0F glycoform (i.e., core glycan) includes an N-acetylglucosamine (GlcNAc) and mannose (Man) core that is modified by a fucosyl (Fuc) residue at the most proximal GlcNAc moiety. The G0 glycoform is identical to the G0F glycoform with the exception that the Fuc moiety of the core glycan is absent. One branch of the N-glycan is referred to as the α(1,6) arm, whereas the other branch is referred to as the α(1,3) arm. The Man5 high mannose glycoform contains a branched glycan structure with 5 mannose moieties attached to the core glycan. The G1F glycoform includes the core glycan with a single terminal galactose (Gal) attached to either the α(1,6) or α(1,3) arm of the branched N-glycan. The G1 glycoform is identical to the G1F glycoform with the exception that the Fuc moiety of the core glycan is absent. The G2F glycoform includes the core glycan with a terminal Gal attached to each of the α(1,6) and α(1,3) arms of the branched N-glycan. The G2 glycoform is identical to the G2F glycoform with the exception that the Fuc moiety of the core glycan is absent. The G2FSA glycoform contains the core G2F glycan structure with a single terminal sialic acid (SA) moiety attached to one of the Gal moieties. The G2FSA2 glycoform contains the core G2F glycan structure with a terminal SA moiety attached to each of the Gal moieties.

FIGS. 2A-2B are bar graphs showing levels of α(2,3) sialylation following transient transfection of CHO cells with a wild-type (WT) and mutated Fc polypeptide (IgG1 isotype) having an alanine (A) substituted for phenylalanine (F) at amino acid position 241 (F241), corresponding to amino acid residue 32 of SEQ ID NO: 2. WT Fc contains predominantly glycans terminating in GlcNAc (G0 or G0F; 49%) or in GlcNAc containing a single Gal (G1 or G1F; 39%), but little to no SA-containing glycoforms (<1.0%; FIG. 2A). F241A mutation results in a dramatic enhancement in the addition of Gal to the Fc domain, with 22% of the molecules containing mono-2,3 SA (G2FSA) and 19% of molecules containing di-2,3 SA (G2FSA2; FIG. 2B).

FIGS. 3A-3D are bar graphs showing N-glycan analysis of the F241A Fc mutant recombinantly expressed by stable transfection of Chinese hamster ovary (CHO) cells using 1 L culture pools. Bar graph showing N-glycan analysis of the F241A Fc mutant expressed solo (Pool 1; FIG. 3A). Bar graph showing N-glycan analysis of the F241A Fc mutant expressed in combination with beta-galactoside alpha-2,6-sialyltransferase 1 (ST6GAL1; Pool 2; FIG. 3B). Bar graph showing N-glycan analysis of the F241A Fc mutant expressed in combination with ST6GAL1 and a beta-1,4-galactosyltransferase 1 (B4GALT1; Pool 3; FIG. 3C). Bar graph showing N-glycan analysis of the F241A Fc mutant expressed in combination with an siRNA targeting the SA transporter, solute carrier family 35 member A1 (SLC35A1 KD; Pool 4; FIG. 3D). Pool 1 is 18% mono-α(2,3) sialylated, 5% di-α(2,3) sialylated, and 47% with one or two terminal Gal. Pool 2 is 30% mono-α(2,6) sialylated, 27% di-α(2,6) sialylated, and 7% with one or two terminal Gal. Pool 3 is 3% mono-α(2,6) sialylated, 81% di-α(2,6) sialylated, and 0% with terminal Gal. Pool 4 is 0% sialylated and 84% with one or two terminal Gal.

FIGS. 4A-4C show blots providing independent verification of glycan structure of Pools 1-4 (P1-P4) from the 1 L stable transfection CHO cultures. Image showing Coomasie staining of each of P1-P4 (FIG. 4A). Blot showing each of P1-P4 stained with biotinylated Sambucus nigra (SNA) lectin to detect α(2,6) SA (FIG. 4B). P1-P4 stained with Maackia amurensis (MAL I) lectin to detect terminal galactose (FIG. 4C).

FIGS. 5A-5F show plots depicting effects of sialylation on F241A Fc mutant exposure in mice, in vivo. These experiments were conducted with F241A Fc material produced by stable transfection of 10 mL CHO cell culture pools. Pool 1 material (F241A alone) contained 15% mono-2,3 sialylation and 20% di-2,3 sialylation. Pool 2 material (F241A+ST6GAL1) contained 31% mono-2,6 sialylation and 35% di-2,6 sialylation. Pool 3 material (F241A+ST6GAL1+B4GALT1) contained 3% mono-2,6 sialylation and 90% di-2,6 sialylation. Pool 4 material (F241A+SLC35A1 KD) contained 0% mono-sialylation and 0% di-sialylation. Plot showing serum concentration (g/mL) of the F241A Fc mutant over time in JAX-014565 mice (mouse FcRn homozygous knock-out, human FcRn hemizygous, Tg32 strain) following intravenous (IV) bolus administration of Pool 2 (F241A+ST6GAL1), Pool 3 (F241A+ST6GAL1+B4GALT1), and Pool 4 (F241A+SLC35A1 KD) with a logarithmically-scaled y-axis (FIG. 5A). Same data as in FIG. 5A shown with a linearly-scaled y-axis (FIG. 5B). The greatest exposure, as measured by area under the curve (AUC), was achieved with the 93% α(2,6) sialylated Pool 3 material. Plot showing serum concentration (g/mL) of the F241A Fc mutant over time in JAX-014565 mice following intravenous (IV) administration of Pool 1 (F241A only), Pool 3 (F241A+ST6GAL1+B4GALT1), and an IgG1 Fc domain (efgartigimod (EFG); Argenx) bearing the ABDEG mutations (M252Y/S254T/T256E/H433K/N434F) to enhance FcRn binding (FIG. 5C) shown with a logarithmically-scaled y-axis. Same data as in FIG. 5C shown with a linearly-scaled y-axis (FIG. 5D). The 93% α(2,6) sialylated Fc-F241A (Pool 3) exhibited the greatest exposure in these human FcRn mice. The ERG Fc domain had the lowest AUC, similar to non-sialylated F241A as shown in FIGS. 5A-5B (Pool 4). Plot showing mean concentration (g/mL) of the F241A Fc mutant over time in male and female CD1 mice following intravenous (IV) administration of Pool 1 (F241A 2,3 sial) or Pool 3 (F241A 2,6 sial)(FIG. 5E). There was no appreciable difference in exposure between male and female mice receiving either α(2,3) sialylated (open and closed triangles) or α(2,6) sialylated (open and closed circles) Fc-F241A. However, the α(2,6) sialylated Fc pool had greater exposure relative to the α(2,3) sialylated pool, although this difference is likely attributable to the extent of sialyation as opposed to the α(2,3) versus α(2,6) linkage. Bar graph showing effects of repeat dosing (100 mg/kg weekly for 4 weeks) of α(2,3) sialylated and α(2,6) sialylated F241A Fc mutant (FIG. 5F) in CD1 mice. Greater exposure at both Cₘᵢₙ (Pre-dose, Day 21) and 24 hours following the 4^{th} dose (24 h Post dose, Day 22) was achieved in male and female CD1 mice following dosing with the α(2,6) sialylated Fc-F241A relative to the α(2,3) sialylated Fc-F241A.

FIGS. 6A-6F show plots depicting therapeutic effects of sialylated F241A Fc polypeptides in a murine model of arthritis. Plot showing clinical scores of K/BxN transgenic mice treated with phosphate buffered saline (PBS), 1 g/kg intravenous immunoglobulin (IVIG), or 50 mg/kg of (41%) α(2,3) sialylated F241A material generated by transient transfection(FIG. 6A). *=difference in clinical score between IVIG and α(2,3) sialylated F241A Fc at day 6 (p=0.002); **=difference in clinical score between IVIG and α(2,3) sialylated F241A Fc at day 7 (p=0.04); for all other days, there was no significant difference between IVIG and α(2,3) sialylated F241A Fc polypeptides. Peak inflammation was achieved by day 7 in the PBS-treated mice. Therefore, clinical scores on study days 7 and 8 are also shown separately in FIGS. 6B-6C, respectively, as box plots represented as mean and standard error of the mean. Both 1 g/kg IVIG and 50 mg/kg F241A Fc significantly suppressed inflammation relative to PBS. These data suggest that F241A is approximately 20-fold more potent than IVIG in the K/BxN serum transfer model of arthritis. Plot showing clinical scores of K/BxN mice treated with PBS, 1 g/kg intravenous immunoglobulin (IVIG), 50 mg/kg of 23% α(2,3) sialylated F241A pool 1 material, or 50 mg/kg of 84% α(2,6) sialylated F241A pool 3 material generated from 1 L stable CHO cultures (FIG. 6D; asterisks represent statistically significant differences as compared to IVIG; p<0.05). IVIG demonstrated significantly greater anti-inflammatory activity than F241A α(2,3) sialylated Fc from day 5 and beyond, as determined by unpaired t-test (p<0.01). By comparison, IVIG was only significantly different than F241A α(2,6) sialylated Fc from days 8 and beyond (unpaired t-test, p<0.05). At the peak of inflammation for PBS-treated animals, days 6 and 7, the α(2,6) sialylated F241A Fc resulted in significantly less inflammation relative to the α(2,3) sialylated F241A Fc (FIGS. 6E-6F).

FIG. 7 shows box-and-whisker plots demonstrating therapeutic efficacy of α(2,3) sialylated F241A in a mouse model of immune thrombocytopenic purpura (ITP) before and after treatment with a 6A6-IgG2a anti-mouse platelet antibody, as indicated by platelet count over time. The 2,3 sialylated Fc-F241A material was equally efficacious to IVIG at 10-fold lower concentration.

FIGS. 8A-8E show plots demonstrating half-life and bioavailability of different F241A Fc glycoforms. (FIG. 8A) Female humanized FcRn mice (n=6 per group) were dosed once with 20 mg/kg of wild-type (WT) Fc or one of the four preparations of F241A Fc: (1) F241A Fc alone; (2) F241A Fc+ST6GAL1; (3) F241A Fc+B4GALT1+ST6GAL1; or (4) F241A Fc+SLC35A1 siRNA. Following dosing, serum concentration of hIgG Fc was measured via ELISA at 1, 3, and 7 days as well as 2, 3, and 5 weeks until serum concentration fell below the detection threshold of 1 μg/ml. From the resulting data, half-life, area under the curve from the time of dosing to the last measurable concentration (AUClast), and clearance rate was calculated for each Fc (see also Table 4). Data are plotted as means with standard deviations. Statistics are an Ordinary One-Way ANOVA with Tukey's multiple comparisons. Correlations between the percent of sialylation on FcF241A and clinical scores (FIG. 8B), half-life (FIG. 8C), AUClast (FIG. 8D), and clearance (FIG. 8E). The trendline in red was generated by a simple linear regression and corresponding R2 and p-values are also shown. The day 7 clinical score correlation was generated from the data obtained in WT mice, while the other correlations were generated from the data in Tg32 mice.

FIGS. 9A-9J are illustrations and plots demonstrating protective effects of the F241A/B4ST6 Fc polypeptide and Fc^{Abdeg} polypeptide (SEQ ID NO: 5) in vivo. (FIG. 9A) Sequence alignment of WT Fc (SEQ ID NO: 1) and Fc^{Abdeg} (SEQ ID NO: 5), with thin boxes designating the locations of the Abdeg mutations, and the thicker box designating the location of the N297 glycosylation site. (FIG. 9B) Illustration of human IgG Fc structure with positions of the Abdeg mutations (empty hexagons) and N297 (striped hexagon) marked, showing their relative distance from each other and different positions on the exterior versus interior of the Fc structure. (FIG. 9C and FIG. 9D) WT Fc, F241A/B4ST6 Fc, and Fc^{Abdeg} were loaded on to anti-human Fc biosensors and binding kinetics to mouse (FIG. 9C) and human FcRn (FIG. 9D) were examined via surface plasmon resonance (SPR). Dissociation constants (KDs) are plotted as μM (solid bars) and association constants (KAs) are plotted as Ms⁻¹ (hatched bars). (FIG. 9E and FIG. 9F) Female WT C57BL/6 (FIG. 9E) and humanized homozygous FcRn (Tg32) mice (FIG. 9F) were administered one dose of 50 mg/kg or 100 mg/kg of F241A/B4ST6 Fc or 10 mg/kg of Fc^{Abdeg} and serum mouse IgG was measured via ELISA out to day 7 post-dose. (FIG. 9G) SIGN-R1^{-/-} and hDC-SIGN⁺ murine bone marrow-derived macrophages (BMDMs) had their Fcγ receptors blocked then were incubated with PBS, F241A/B4ST6 Fc, or Fc^{Abdeg} and binding was detected by FACS using an anti-human IgG Fc antibody. Plot shows the percentage of cells bound by the added Fc. (FIG. 911-9J) Female WT C57BL/6 mice (n=5 per group) and female and male SIGN-R1^{-/-} mice (n=5 per group) were administered arthritogenic K/BxN serum alongside PBS, 1 g/kg IVIG, 50 mg/kg F241A/B4ST6 Fc, or 10 mg/kg Fc^{Abdeg} in a preventative manner, and joint swelling was clinically scored for 10 days. (FIG. 911) Clinical scores of joint inflammation shown across 10 days in female WT C57BL6 mice (n=5 mice per group). (FIG. 9I) Clinical scores of joint inflammation shown across 10 days in female SIGN-R1^{-/-} mice (n=5 mice per group). (FIG. 9J) Day 7 clinical scores of joint inflammation for each group, representing the peak of disease, are plotted. Bar graphs are plotted as means with standard deviations. Statistical significance was assessed using an ordinary one-way ANOVA with Tukey's multiple comparisons.

FIGS. 10A-10H are illustrations and plots showing combinatorial prophylactic and therapeutic effects of F24l/B4ST6 Fc and Fc^{Abdeg} in a murine model of arthritis in vivo. (FIG. 10A) Female WT C57BL/6 mice (n=5) were administered arthritogenic K/BxN serum on day 0, followed by PBS, 1 g/kg IVIG, 50 mg/kg F241A/B4ST6 Fc, or 10 mg/kg Fc^{Abdeg} in a therapeutic manner on day 2. Joint swelling was clinically scored for 12 days post-K/BxN. (FIG. 10B) Clinical scores of joint inflammation shown across 12 days in female WT C57BL6 mice (n=4 or 5 mice per group). (FIG. 10C) Bar graph showing day 8 (peak of disease) clinical scores for each of the aforementioned groups. (FIG. 10D) Female WT C57BL/6 mice (n=4-5) were administered 50 mg/kg F241A/B4ST6 Fc alone or combined with increasing doses of Fc^{Abdeg} (1-10 mg/kg). After 10 days, serum human IgG Fc was detected via ELISA. (FIG. 10E) Female WT C57BL/6 mice (n=3-5) were administered arthritogenic K/BxN serum alongside PBS, 1 g/kg IVIG, 50 mg/kg F241A/B4ST6 Fc, 1 mg/kg Fc^{Abdeg} or a combination of F241A/B4ST6 Fe and Fc^{Abdeg} in a prophylactic manner, and joint swelling was clinically scored for 10 days. Clinical scores of joint inflammation are shown across 10 days in female WT C57BL6 mice (n=3-5 mice per group). (FIG. 10F) Day 6 clinical scores for each group, representing the peak of disease, are plotted. (FIG. 10G) Female WT C57BL/6 mice (n=5) were administered arthritogenic K/BxN serum on day 0, followed by PBS, 1 g/kg IVIG, 50 mg/kg F241A/B4ST6 Fc, 1 mg/kg Fc^{Abdeg} or a combination of F241A/B4ST6 Fc and Fc^{Abdeg} in a therapeutic manner on day 2. Joint swelling was clinically scored for 12 days post-K/BxN. Clinical scores of joint inflammation are shown across 12 days in female WT C57BL6 mice (n=5 mice per group) (FIG. 10H) Day 9 clinical scores for each group, representing the peak of disease, are plotted. Bar graphs are plotted as means with standard deviations. Statistical significance was assessed using an ordinary one-way ANOVA with Tukey's multiple comparisons.

FIGS. 11A and 11B depict skin blistering scores and FcγRIIB expression in peripheral neutrophils. FIG. 11A downward triangles indicate days treatments were administered (day 0 (3 hours prior to administration of pathogenic autoantibody) and day 3). The percent of total skin area affected by blisters (y-axis±SEM) is plotted over days post disease induction (x-axis). Open circles, squares, and upright triangles depict treatment with PBS, 100 mg/kg Fc^{F241A}, or 10 mg/kg efgartigimod, respectively. Four animals were used per group. FIG. 11B depicts FcgRIIB expression on the surface of peripheral neutrophils (MFI SEM; n=4 per group) in mice treated with PBS, Fc^{F241A}, or efgartigimod. Statistical significance was determined by two-way ANOVA, where Fc^{F241A} uniquely resulted in a significant (p<0.001) increase in FcγRIIB expression on study day 5 relative to PBS.

FIGS. 12A-12C show the results of fluorescence-activated cell sorting (FACS) analysis of the kinetics of FcγRIIB upregulation on B cells and neutrophils. Shown in FIGS. 12A, 12B, and 12C are FACS plots assessing FcγRIIB expression (MFI±SEM; n=4 per group) on the surfaces of peripheral naïve B cells, mature B cells, and neutrophils, respectively, in mice treated with PBS, NVG-2089, or efgartigimod. Data are plotted over days post epidermolysis bullosa acquisita (EBA) induction on the x-axis. Statistical significance was determined by two-way ANOVA using multiple comparisons.

FIG. 13 shows images of tissue sections from hematoxylin and eosin (H&E) stained ear tissue biopsies. Skin samples from areas of the mouse ear affected by blisters were biopsied and H&E stained for infiltrating immune cells. The top, middle, and bottom rows depict images from mice treated with vehicle control, Fc^{F241A}, and efgartigimod, respectively. The images are annotated to reveal cartilage, blood, dead skin, and blisters.

FIGS. 14A and 14B shows results of experiments to assess changes in immune cell populations present in skin biopsies. The average percent (+SEM) of neutrophils (FIG. 14A) and CD62L high expressing monocytes (FIG. 14B) present in the ear skin lesions are shown on the y-axis, plotted over days after epidermolysis bullosa acquisita (EBA) induction on the x-axis. Statistical significance was determined by two-way ANOVA using multiple comparisons.

FIG. 15 is a graph which shows body weight of mice (mean+SEM normalized to study day 0) (shown on the y-axis) after experimental autoimmune encephalomyelitis (EAE) induction plotted against days following treatment initiation (shown on the x-axis) for each treatment group (n=7). Treatment groups are as shown in the figure legend. Statistical significance relative to vehicle control group is indicated by asterisks and determined using a two-way ANOVA.

FIG. 16A is a graph that shows clinical scores (mean+SEM) (shown on the y-axis) plotted against days following treatment initiation (shown on the x-axis) for each treatment group (n=7). FIG. 16B is a bar graph which shows the mean AUC of clinical scores (+SEM) over the entire time-course of the study. Treatment groups are defined in the figure legend. Statistical significance relative to vehicle control group is indicated by asterisks and determined using a two-way or one-way ANOVA, as indicated.

### DETAILED DESCRIPTION

Inflammatory disorders, including autoimmune diseases, are disorders involving abnormal activation and subsequent migration of white blood cells to affected areas of the body. These conditions encompass a wide range of ailments that affect the lives of millions of people throughout the world. Although various treatments are presently available, many possess significant side effects or are insufficiently effective in alleviating symptoms.

Immunoglobulin G (IgG) has long been appreciated to mediate both pro- and anti-inflammatory activities through interactions mediated by its fragment crystallizable (Fc) region. While Fc-FcγR interactions are responsible for the pro-inflammatory properties of immune complexes and cytotoxic antibodies, intravenous gamma globulin (IVIG) and its constituent Fc fragments are anti-inflammatory and are widely used to suppress inflammation in disease states. It has been proposed that glycosylation of IgG is crucial for regulation of its cytotoxicity and inflammatory potential. For example, prior studies have demonstrated that anti-inflammatory activity of IVIG is a property of the Fc fragment and its linked N-glycan sialic acid (SA) moieties, indicating a combined requirement for a specific polypeptide backbone and glycosylation profile for its anti-inflammatory effect.

Sialylation of recombinant therapeutic glycoproteins, such as Fc polypeptides, is generally performed in mammalian cell lines capable of reproducing mammalian or mammalian-like glycosylation profiles. Addition of terminal SA residues on the N-glycan of the Fc heavy chain, particularly at Asparagine 297 (Asn297), has been shown to impact various in vivo parameters of the Fc polypeptide, including absorption, blood half-life and clearance, as well as its immunogenic or immunosuppressive properties. Glycoengineering of Fc polypeptides with high levels of sialylation has faced significant obstacles, including limited and non-homogenous sialylation of pools of said polypeptides.

Disclosed herein, in some embodiments, are modified and highly sialylated Fc polypeptides having an amino acid substitution from phenylalanine (F) to an aliphatic amino acid residue (e.g., alanine, glycine, isoleucine, leucine, proline, valine, and methionine) at amino acid position 241 of the Fc heavy chain (F241A; numbered according to Kabat). Additionally disclosed are methods of manufacturing the modified Fc polypeptides using a recombinant expression system that includes one or more (e.g., 1, 2, 3, or more) nucleic acid expression vectors and mammalian host cells. Furthermore, the present disclosure provides methods for treating an inflammatory disease or disorder (e.g., an autoimmune disorder, such as arthritis or idiopathic thrombocytopenic purpura (ITP)) using a therapeutic agent or composition disclosed herein. A subject with the inflammatory disease or disorder is treated in accord with the methods disclosed herein by administering the therapeutic agent or composition to the subject by any acceptable route.

### Immunoglobulin and Fc Glycosylation

IgG is a glycoprotein composed of two identical heavy chains and two light chains which are composed of variable and constant domains. IgG contains a single, N-linked glycan at asparagine 297 (Asn297) in the CH2 domain on each of its two heavy chains. The covalently-linked, complex carbohydrate is composed of a core biantennary penta-saccharide containing N-acetylglucosamine (GlcNAc) and mannose (Man). Further modification of the core carbohydrate structure is observed in serum antibodies with the presence of fucose (Fuc), branching GlcNAc, galactose (Gal) and variably present terminal sialic acid (SA) moieties. Over 40 different glycoforms have thus been detected to be covalently attached to this single glycosylation site. Glycosylation of IgG has been shown to be essential for binding to all FcγRs by maintaining an open conformation of the two heavy chains. It is believed that this IgG glycosylation for FcγR binding accounts for the inability of de-glycosylated IgG antibodies to mediate in vivo triggered inflammatory responses, such as antibody-dependent cellular cytotoxicity (ADCC), phagocytosis, and release of inflammatory mediators. That individual IgG glycoforms may contribute to modulation of inflammatory responses has been suggested by the altered affinities for individual FcγRs reported for IgG antibodies containing or lacking Fuc and its consequential effects on cytotoxicity. A link between autoimmune states and specific glycosylation patterns of IgG antibodies has been observed in patients with rheumatoid arthritis and autoimmune vasculitis in which decreased galactosylation and sialylation of IgG antibodies have been reported.

### Compositions

Disclosed herein, in some embodiments, are compositions (e.g., therapeutic compositions) comprising modified Fc polypeptides having sequences of variants of a wild-type human IgG Fc polypeptide of SEQ ID NO: 1 and having high levels of sialylation, such as at least 60% α(2,6) sialylation (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) or about 40% α(2,3) sialylation (e.g., 30%, 31%, 32%, 33%, 34%, 35%, 36%, 37%, 38%, 39%, 40%, 41%, 42%, 43%, 44%, 45%, 46%, 47%, 48%, 49%, 50%). Also disclosed are pharmaceutical compositions comprising the modified Fc polypeptides and a pharmaceutically acceptable carrier, excipient, or diluent.

### Fc Polypeptide Variants

In some embodiments, the compositions described herein include a variant (i.e., modified) Fc polypeptide (e.g., IgG1 Fc polypeptide) containing one or more (e.g., 1, 2, 3, 4, 5, 6, 7, 8, 9, 10, or more) amino acid substitutions relative to its wild-type/parental amino acid sequence of SEQ ID NO: 1 (bolded and underlined phenylalanine residue (N) corresponds to Asn297, to which the Fc N-glycan is attached; numbered according to the Kabat system).

(SEQ ID NO: 1)
KVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFF
LYSKLTVDKSRWQQGNVFSCSVMHEALHNHYTQKSLSLSPGK

In some embodiments, the one or more amino acid substitutions is at a phenylalanine (Phe; F) at position 241 of the Fc polypeptide (corresponding to amino acid position 32 of SEQ ID NO: 1; numbered according to the Kabat system). In some embodiments, the Phe at position 241 of the modified Fc polypeptide is substituted for an aliphatic amino acid residue (e.g., alanine, glycine, valine, leucine, isoleucine, and proline). In some embodiments, the Phe at position 241 of the modified Fc polypeptide is substituted for an alanine (Ala or A; F241A substitution). In some embodiments, the modified Fc polypeptide includes the F241A substitution and has the amino acid sequence of SEQ ID NO: 2 or is a variant thereof having at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) sequence identity to SEQ ID NO: 2. In some embodiments, the modified Fc polypeptide includes the F241A substitution and has an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) sequence identity to SEQ ID NO: 2. In some embodiments, the modified Fc polypeptide includes the F241A substitution and has an amino acid sequence having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 2. In some embodiments, the modified Fc polypeptide includes the F241A substitution and has an amino acid sequence having at least 90% (e.g., at least 90%, 91%, 92%, 93%, 94%, 95%, 96%, 97%, 98%, 99%, or more) sequence identity to SEQ ID NO: 2. In some embodiments, the modified Fc polypeptide includes the F241A substitution and has an amino acid sequence having at least 95% (e.g., at least 95%, 96%, 97%, 98%, 99%, or more) sequence identity to SEQ ID NO: 2. In some embodiments, the modified Fc polypeptide includes the F241A substitution and has an amino acid sequence having at least 98% (e.g., at least 98%, 99%, or more) sequence identity to SEQ ID NO: 2. In some embodiments, the modified Fc polypeptide includes the F241A substitution (shown as a bolded and underlined A, below) and has an amino acid sequence of SEQ ID NO: 2, as shown below.

(SEQ ID NO: 2)
KVDKKVEPKSCDKTHTCPPCPAPELLGGPSVALFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFF
LYSKLTVDKSRWQQGNVFSCSVMHEALHNHYTQKSLSLSPGK

In some embodiments, the modified Fc polypeptide includes the F241A substitution and has the amino acid sequence of SEQ ID NO: 6 or is a variant thereof having at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) sequence identity to SEQ ID NO: 6. In some embodiments, the modified Fc polypeptide includes the F241A substitution and has an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) sequence identity to SEQ ID NO: 6. In some embodiments, the modified Fc polypeptide includes the F241A substitution and has an amino acid sequence having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 6. In some embodiments, the modified Fc polypeptide includes the F241A substitution and has an amino acid sequence having at least 90% (e.g., at least 90%, 91%, 92%, 93%, 94%, 95%, 96%, 97%, 98%, 99%, or more) sequence identity to SEQ ID NO: 6. In some embodiments, the modified Fc polypeptide includes the F241A substitution and has an amino acid sequence having at least 95% (e.g., at least 95%, 96%, 97%, 98%, 99%, or more) sequence identity to SEQ ID NO: 6. In some embodiments, the modified Fc polypeptide includes the F241A substitution and has an amino acid sequence having at least 98% (e.g., at least 98%, 99%, or more) sequence identity to SEQ ID NO: 6. In some embodiments, the modified Fc polypeptide includes the F241A substitution (shown as a bolded and underlined A, below) and has an amino acid sequence of SEQ ID NO: 6, as shown below.

(SEQ ID NO: 6)
KVDKRVEPKSCDKTHTCPPCPAPELLGGPSVALFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFF
LYSKLTVDKSRWQQGNVFSCSVMHEALHNHYTQKSLSLSPGK

The amino acid composition of a modified Fc polypeptide described herein vary without disrupting the ability of the polypeptide to bind to the respective receptor and trigger the respective cellular response, in some embodiments. For example, it contains one or more conservative amino acid substitutions, in some embodiments. A “conservative amino acid substitution” is one in which the amino acid residue is replaced with an amino acid residue having a similar side chain. Families of amino acid residues having similar side chains are known. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in, e.g., SEQ ID NO: 2, is preferably replaced with another amino acid residue from the same side chain family. Alternatively, mutations can be introduced randomly along all or part of the sequences, such as by saturation mutagenesis, and the resultant mutants can be screened for the ability to bind to the respective Fc receptor and trigger a corresponding cellular response to identify mutants that retain the activity as desired.

In some embodiments, the modified Fc polypeptide is IgG1 isotype. In some embodiments, the modified Fc polypeptides is IgG3 isotype.

### Sialylation of Modified Fc Polypeptides

Glycosylation of immunoglobulins has been shown to have significant effects on their effector functions, structural stability, and rate of secretion from antibody-producing cells. The carbohydrate groups responsible for these properties are generally attached to the constant (C) regions of the antibodies. For example, glycosylation of IgG at Asn297 in the CH2 domain is required for the full capacity of IgG to activate the classical pathway of complement-dependent cytolysis.

Each antibody possesses a distinct array of N-linked glycan structures which variably affect protein assembly, secretion, and function. These N-linked glycans vary considerably, depending on the degree of processing, and include high-mannose, as well as complex biantennary oligosaccharides with or without bisecting GlcNAc and core fucose (Fuc) residues, in some embodiments. Typically, there is heterogeneous processing of the core oligosaccharide structures attached at a particular glycosylation site such that even monoclonal antibodies exist as multiple glycoforms. Likewise, it has been shown that major differences in antibody glycosylation occur between antibody-producing cell lines, and even minor differences are seen for a given cell line grown under different culture conditions.

The presence of sialic acid (SA) residues on the N-glycan of Fc polypeptides has recently been identified as an important factor in mediating anti-inflammatory effects of intravenous immunoglobulin (IVIG), which has been demonstrated in certain autoimmune diseases. It has been proposed that this anti-inflammatory effect is mediated by binding of sialylated Fc components of IVIG to inhibitory FcγRIIB receptors and DC-SIGN receptors. Removal of the IVIg sialic acid results in loss of protection in animal models of multiple sclerosis, rheumatoid arthritis, Guillain-Barre syndrome, and idiopathic thrombocytopenic purpura (ITP), despite retaining normal circulating half-life and binding to FcRn. Conversely, hyper-sialylation of IVIg increases the potency of anti-inflammatory activity 10-30-fold in several different animal models of autoimmune disease. The biological consequence of binding to and activating the type II Fc receptors is IL33 release, inhibitory FcγRIIB upregulation, and T regulatory cell expansion. In mice, knock-out of SIGNR1 (the murine homologue of DC-SIGN) blocks the anti-inflammatory activity of IVIg. Blocking the IL33 receptor, knockout of FcγRIIB, or depletion of T regulatory cell, in different contexts, also blocks the anti-inflammatory properties of IVIg. However, only a minor portion of IgG in IVIG have glycans terminating in SA, thereby requiring administration of IVIG at high doses (1-2 g/kg) to elicit a therapeutic anti-inflammatory effect. Previous efforts to produce highly sialylated pools of IgG Fc focused on purifying sialylated fractions of Fc polypeptides from IVIG. However, there are no available methods for recombinantly producing pure, highly sialylated Fc peptides having predetermined levels of sialylation.

The present disclosure provides methods for enhancing sialylation of IgG Fc polypeptides by providing modified Fc polypeptides (e.g., an F241A Fc mutant of SEQ ID NO: 2 or a variant thereof) in a recombinant expression system (e.g., one or more nucleic acid expression vectors introduced into a host cell, such as a mammalian host cell) alone or in combination with one or more (e.g., 1, 2, or more) recombinant glycosyltransferase enzymes (e.g., ST6GAL1 and B4GALT1) under conditions and for a time sufficient to yield desired levels of Fc sialylation. In some embodiments, the one or more recombinant glycosyltransferase enzymes is ST6GAL1. Activity of ST6GAL1 results in 6-sialylated oligosaccharides, including 6-sialylated galactose. The term “ST6GAL1” refers to a sialyltransferase enzyme capable of attaching SA to the sixth atom of the acceptor polysaccharide. In some embodiments, the one or more recombinant glycosyltransferase enzymes is B4GALT1. The term “B4GALT1” refers to an enzyme belonging to a family of beta-1,4-galactosyltransferases that transfers galactose in a β(1,4) linkage to acceptor sugars, such as GlcNAc, Glc, and Xyl. In some embodiments, the one or more recombinant glycosyltransferase enzymes are ST6GAL1 and B4GALT1.

The disclosed methods are used to achieve specified levels of Fc sialylation. In some embodiments, modified Fc polypeptides of the disclosure are expressed and/or cultured under conditions and for a time sufficient to produce a pool of Fc polypeptides having at least 40%, 45%, 50%, 55%, 60%, 65%, 70%, 75%, 80%, 85%, 90%, 95%, or about 100% sialylated Fc polypeptides within the pool. In some embodiments, the modified Fc polypeptides are expressed and/or cultured under conditions and for a time sufficient to produce a pool of Fc polypeptides having at least 40% sialylated Fc polypeptides within the pool. In some embodiments, the modified Fc polypeptides are expressed and/or cultured under conditions and for a time sufficient to produce a pool of Fc polypeptides having at least 45% sialylated Fc polypeptides within the pool. In some embodiments, the modified Fc polypeptides are expressed and/or cultured under conditions and for a time sufficient to produce a pool of Fc polypeptides having at least 50% sialylated Fc polypeptides within the pool. In some embodiments, the modified Fc polypeptides are expressed and/or cultured under conditions and for a time sufficient to produce a pool of Fc polypeptides having at least 55% sialylated Fc polypeptides within the pool. In some embodiments, the modified Fc polypeptides are expressed and/or cultured under conditions and for a time sufficient to produce a pool of Fc polypeptides having at least 60% sialylated Fc polypeptides within the pool. In some embodiments, the modified Fc polypeptides are expressed and/or cultured under conditions and for a time sufficient to produce a pool of Fc polypeptides having at least 65% sialylated Fc polypeptides within the pool. In some embodiments, the modified Fc polypeptides are expressed and/or cultured under conditions and for a time sufficient to produce a pool of Fc polypeptides having at least 70% sialylated Fc polypeptides within the pool. In some embodiments, the Fc polypeptides are expressed and/or cultured under conditions and for a time sufficient to produce a pool of Fc polypeptides having at least 75% sialylated Fc polypeptides within the pool. In some embodiments, the modified Fc polypeptides are expressed and/or cultured under conditions and for a time sufficient to produce a pool of Fc polypeptides having at least 80% sialylated Fc polypeptides within the pool. In some embodiments, the modified Fc polypeptides are expressed and/or cultured under conditions and for a time sufficient to produce a pool of Fc polypeptides having at least 85% sialylated Fc polypeptides within the pool. In some embodiments, the modified Fc polypeptides are expressed and/or cultured under conditions and for a time sufficient to produce a pool of Fc polypeptides having at least 90% sialylated Fc polypeptides within the pool. In some embodiments, the modified Fc polypeptides are expressed and/or cultured under conditions and for a time sufficient to produce a pool of Fc polypeptides having at least 95% sialylated Fc polypeptides within the pool. In some embodiments, the modified Fc polypeptides are expressed and/or cultured under conditions and for a time sufficient to produce a pool of Fc polypeptides having about 100% sialylated Fc polypeptides within the pool. In some embodiments, the modified Fc polypeptides are expressed and/or cultured under conditions and for a time sufficient to produce a pool of Fc polypeptides having between 40% and 100% sialylated Fc polypeptides within the pool, such as between 45% and 50%, between 50% and 55%, between 55% and 60%, between 60% and 65%, between 65% and 70%, between 70% and 75%, between 75% and 80%, between 80% and 85%, between 85% and 90%, between 90% and 95%, or between 95% and 100%.

In some embodiments, the modified Fc polypeptides are expressed and/or cultured under conditions and for a time sufficient to produce a pool of modified Fc polypeptides including at least 60% (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the Fc polypeptides having an SA moiety attached to an N-glycan of the Fc polypeptide via an α(2,6) linkage. In some embodiments, at least 65% (e.g., at least 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the modified Fc polypeptides comprise the SA moiety attached to the N-glycan of the Fc polypeptides via the α(2,6) linkage. In some embodiments, at least 70% (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the modified Fc polypeptides comprise the SA moiety attached to the N-glycan of the Fc polypeptides via the α(2,6) linkage. In some embodiments, at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the modified Fc polypeptides comprise the SA moiety attached to the N-glycan of the Fc polypeptides via the α(2,6) linkage. In some embodiments, at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) of the modified Fc polypeptides comprise the SA moiety attached to the N-glycan of the Fc polypeptides via the α(2,6) linkage. In some embodiments, at least 85% (e.g., at least 85%, 86%, 87%, 88%, 89%, 90%, 91%, 92%, 93%, 94%, 95%, 96%, 97%, 98%, 99%, or more) of the modified Fc polypeptides comprise the SA moiety attached to the N-glycan of the Fc polypeptides via the α(2,6) linkage. In some embodiments, at least 90% (e.g., at least 90%, 91%, 92%, 93%, 94%, 95%, 96%, 97%, 98%, 99%, or more) of the modified Fc polypeptides comprise the SA moiety attached to the N-glycan of the Fc polypeptides via the α(2,6) linkage. In some embodiments, at least 95% (e.g., at least 95%, 96%, 97%, 98%, 99%, or more) of the modified Fc polypeptides comprise the SA moiety attached to the N-glycan of the Fc polypeptides via the α(2,6) linkage. In some embodiments, at least 98% (e.g., at least 98%, 99%, or more) of the modified Fc polypeptides comprise the SA moiety attached to the N-glycan of the Fc polypeptides via the α(2,6) linkage. In some embodiments, at least 99% of the modified Fc polypeptides comprise the SA moiety attached to the N-glycan of the Fc polypeptides via the α(2,6) linkage. In some embodiments, about 100% of the modified Fc polypeptides comprise the SA moiety attached to the N-glycan of the Fc polypeptides via the α(2,6) linkage. In some embodiments, the N-glycan of the modified Fc polypeptides is mono-sialylated or di-sialylated. In some embodiments, at least 30% of the modified Fc polypeptides comprise mono-sialylated N-glycans comprising a SA moiety attached via the α(2,6) linkage. In some embodiments, at least 30% of the modified Fc polypeptides comprise di-sialylated N-glycans comprising two SA moieties attached via the α(2,6) linkage. In some embodiments, about 90% of the modified Fc polypeptides comprise di-sialylated N-glycans comprising two SA moieties attached via the α(2,6) linkage.

In some embodiments, at least about 60% (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the modified Fc polypeptides comprise a galactose moiety. In some embodiments, at least about 70% (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the modified Fc polypeptides comprise a galactose moiety. In some embodiments, at least about 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) of the modified Fc polypeptides comprise a galactose moiety. In some embodiments, at least about 90% (e.g., at least 90%, 91%, 92%, 93%, 94%, 95%, 96%, 97%, 98%, 99%, or more) of the modified Fc polypeptides comprise a galactose moiety. In some embodiments, about 100% of the modified Fc polypeptides comprise a galactose moiety. In some embodiments, the galactose moiety is attached to an α(1,3) arm and/or α(1,6) arm of the N-glycan. In some embodiments, the galactose moiety is a branched galactose moiety.

In some embodiments, the modified Fc polypeptides are expressed and/or cultured under conditions and for a time sufficient to produce a pool of modified Fc polypeptides including about 40% (e.g., 30%, 31%, 32%, 33%, 34%, 35%, 36%, 37%, 38%, 39%, 40%, 41%, 42%, 43%, 44%, 45%, 46%, 47%, 48%, 49%, or 50%) of the Fc polypeptides having an SA moiety attached to an N-glycan of the modified Fc polypeptide via an α(2,3) linkage.

In some embodiments, the N-glycan is attached to the asparagine (Asn) at amino acid residue 297 of the polypeptide (Asn297; numbered according to Kabat; corresponding to amino acid residue 88 of SEQ ID NO: 2).

### Methods of Treatment

### Selection of Subjects

Disclosed herein, in some embodiments, are methods of treating or preventing an inflammatory disease or disorder in an individual in need thereof. The term “inflammatory disease or disorder” refers to a disease or disorder characterized by abnormal or undesirable inflammation (e.g., an autoimmune disease or disorder). In some embodiments, the autoimmune disease or disorder is a T cell-mediated autoimmune disease or disorder (e.g., multiple sclerosis and type I diabetes). The term “T cell-mediated disease” refers to any inflammatory disorder characterized by an abnormal low level of functionally active regulatory T (Treg) cells or abnormally activated effector T cells. In some embodiments, the autoimmune disease or disorder is arthritis (e.g., rheumatoid arthritis and psoriatic arthritis). In some embodiments, the autoimmune disease or disorder is idiopathic thrombocytopenic purpura (ITP).

It has been proposed that glycosylation of IgG is crucial for regulation of its cytotoxicity and inflammatory potential. For example, anti-inflammatory activity of IVIG has been shown to be a property of the Fc fragment and terminal SA residues on its linked N-glycan, indicating a combined requirement for a specific polypeptide backbone and glycosylation profile for its anti-inflammatory effect. Surprisingly, the disclosed compositions containing high levels of α(2,6) (e.g., at least 60%) or α(2,3) sialylation (e.g., about 40%) were shown by the present inventors to have a robust therapeutic effect in murine models of arthritis and ITP, thereby underscoring potential clinical efficacy of the disclosed compositions for treatment of inflammatory diseases or disorders.

The compositions and methods described herein are used to treat patients presenting with symptoms of the inflammatory disease or disorder, in some embodiments. The compositions and methods described herein may also be used to treat patients that are in remission from the inflammatory disease or disorder. Furthermore, the compositions and methods described herein may also be administered as a preventative treatment to patients at risk of developing the inflammatory disease or disorder.

### Routes of Administration

The modified Fc polypeptides described herein or compositions containing the same are, in some embodiments, administered to a subject with an inflammatory disease or disorder by a variety of routes, such as intravenously, intradermally, subcutaneously, percutaneously, transdermally, intramuscularly, transmucosally, or intraosseously. In some embodiments, the compositions described herein are administered to a subject systemically (e.g., intravenously). In some embodiments, the compositions described herein are administered to the subject locally (e.g., to the site of inflammation). The most suitable route for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. Multiple routes of administration may be used to treat a single subject. Multiple routes of administration may be used to treat a single subject at one time, or the subject may receive treatment via one route of administration first, and receive treatment via another route of administration during a second appointment, e.g., 1 week later, 2 weeks later, 1 month later, 6 months later, or 1 year later. Compositions of the disclosure may be administered to a subject once, or two or more times (e.g., 2-10 times) per week, month, or year to a subject for treatment.

### Dosage

The amount of modified Fc polypeptides disclosed herein or compositions containing the same used with the methods of treatment disclosed herein will typically be a therapeutically effective amount. As a non-limiting example, an effective amount is an amount sufficient to reduce a symptom of an inflammatory disease or disorder, including, e.g., edema, hyperemia, erythema, bruising, tenderness, stiffness, swollenness, fever, chills, stuffy nose, stuffy head, breathing difficulties, fluid retention, blood clots, loss of appetite, increased heart rate, formation of granulomas, fibrinous, pus, non-viscous serous fluid, ulcer, increased production of self-reactive effector immune cells, inflammation, or pain.

The appropriate effective amount of a composition to be administered for a particular application of the disclosed methods can be determined by, e.g., using the guidance provided herein. For example, the effectiveness of a composition disclosed herein in treating a symptom of an inflammatory disease or disorder is determined by observing one or more clinical symptoms, and/or physiological indicators associated with the condition, in some embodiments. The response of an individual with an inflammatory disease or disorder to treatment may be monitored by determining the severity of their symptoms or by determining the frequency of autoreactive T cells in a sample from an individual with the inflammatory disease or disorder (e.g., autoimmune disease or disorder). The severity of symptoms of the autoimmune disease or disorder may correlate with the number of autoreactive T cells. In addition, an increase in the number of autoreactive T cells in the sample may be used as an indication to apply treatments intended to minimize the severity of the symptoms and/or treat the autoimmune disease or disorder before the symptoms appear. As another example, the effectiveness of a composition disclosed herein in treating a symptom of an autoimmune disease or disorder disclosed herein can be determined by relying on the clinical experience with existing T cell infusion therapies. An improvement in an autoimmune disease or disorder also can be indicated by a reduced need for a concurrent therapy. Those of skill in the art will know the appropriate symptoms or indicators associated with a specific autoimmune disease or disorder and will know how to determine if an individual is a candidate for treatment as disclosed herein. The condition of the individual can be monitored throughout the course of therapy and that the effective amount of a compound or composition disclosed herein that is administered can be adjusted accordingly.

In some embodiments, a therapeutically effective amount of a composition disclosed herein reduces a symptom associated with an inflammatory disease or disorder by, e.g., at least 10%, at least %, at least 30%, at least 40%, at least 50%, at least 60%, at least 70%, at least 80%, at least 90% or at least 100%. In some embodiments, a therapeutically effective amount of a composition disclosed herein reduces a symptom associated with an inflammatory disease or disorder by, e.g., at most 10%, at most 20%, at most 30%, at most 40%, at most 50%, at most 60%, at most 70%, at most 80%, at most % or at most 100%. In some embodiments, a therapeutically effective amount of a composition disclosed herein reduces a symptom associated with an inflammatory disease or disorder by, e.g., about 10% to about 100%, about 10% to about 90%, about 10% to about 80%, about 10% to about %, about 10% to about 60%, about 10% to about 50%, about 10% to about 40%, about 20% to about 100%, about 20% to about 90%, about 20% to about 80%, about 20% to about 20%, about 20% to about %, about 20% to about 50%, about 20% to about 40%, about 30% to about 100%, about 30% to about %, about 30% to about 80%, about 30% to about 70%, about 30% to about 60%, or about 30% to about 50%. In some embodiments, a therapeutically effective amount of a composition disclosed herein is a dosage sufficient to reduces a symptom associated with an inflammatory disease or disorder for, e.g., at least one week, at least one month, at least two months, at least three months, at least four months, at least five months, at least six months, at least seven months, at least eight months, at least nine months, at least ten months, at least eleven months, at least twelve months, or more.

### Therapeutic Effects

In some embodiments, a composition of the disclosure is administered in an amount and for a time effective to result in reduction in one or more (e.g., 1 or more, 2 or more, 3 or more, 4 or more, 5 or more, 6 or more, 7 or more, 8 or more, 9 or more, or 10 or more) of: decrease immune cell (e.g., T cell, B cell, NK cell, ILC1, ILC2, ILC3, monocyte, macrophage (M1 and M2), dendritic cell, or antigen presenting cell) migration, decrease immune cell proliferation, decrease immune cell recruitment, increase immune cell lymph node homing, decrease immune cell lymph node egress, decrease immune cell differentiation, decrease immune cell activation, decrease immune cell polarization, decrease immune cell cytokine production, decrease immune cell degranulation, decrease immune cell maturation, decrease immune cell ADCC, decrease immune cell ADCP, decrease immune cell antigen presentation, reduce immune cell serotonin receptor expression, treat the inflammatory disease or disorder, reduce symptoms of an inflammatory disease or disorder, reduce inflammation, reduce auto-antibody levels, increase organ function, and decrease rate or number of relapses or flare-ups.

Reduction in the severity of the aforementioned symptoms is, in some embodiments, by any amount, so long as a therapeutic benefit is achieved in the patient. Treatment efficacy is measured across different timeframes, including e.g., in months to years, depending on prognostic factors including the number of relapses, stage of disease, and other factors.

Prolonging survival is another desired treatment benchmark that includes, without limitation, an increase in survival time by at least 1 month (mo), about at least 2 months (mos), about at least 3 mos, about at least 4 mos, about at least 6 mos, about at least 1, 2, 3, 4, 5, 6, 7, 8, 9, 10, 20, 30, 40, 50 years, or more. In some embodiments, overall survival is measured in months to years. In some embodiments, the subject's symptoms remain static or decrease.

### Combination Therapy

The compositions disclosed herein are, in some embodiments, administered in combination with one or more (e.g., 1, 2, 3, 4, 5 or more) additional therapeutic agents or modalities for treatment of a disease or disorder described herein (e.g., an inflammatory disease or disorder, such as an autoimmune disease or disorder).

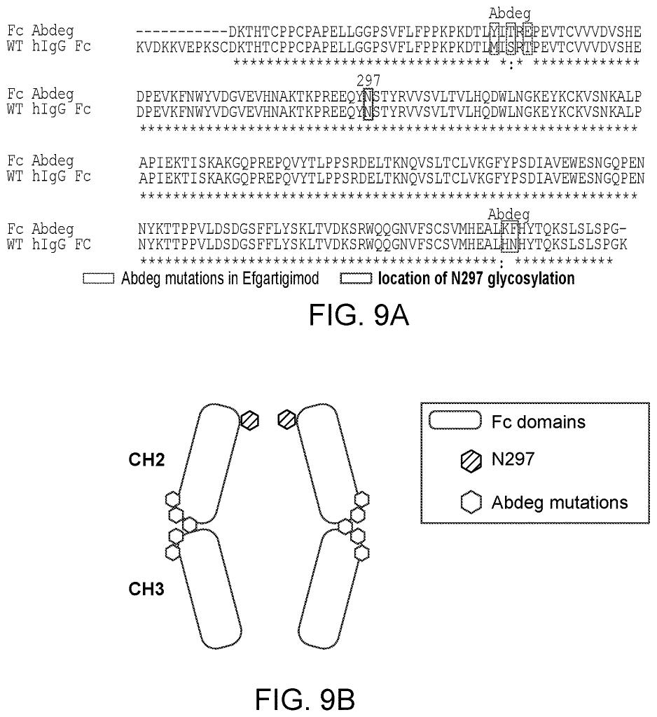

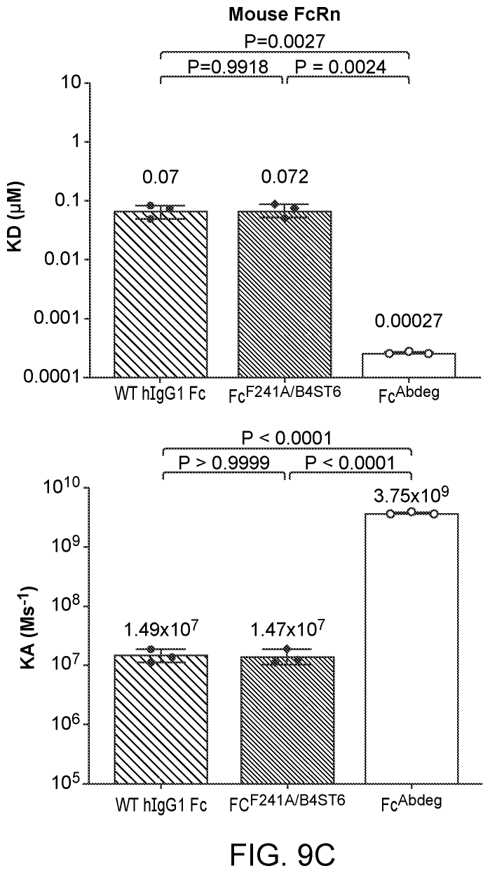

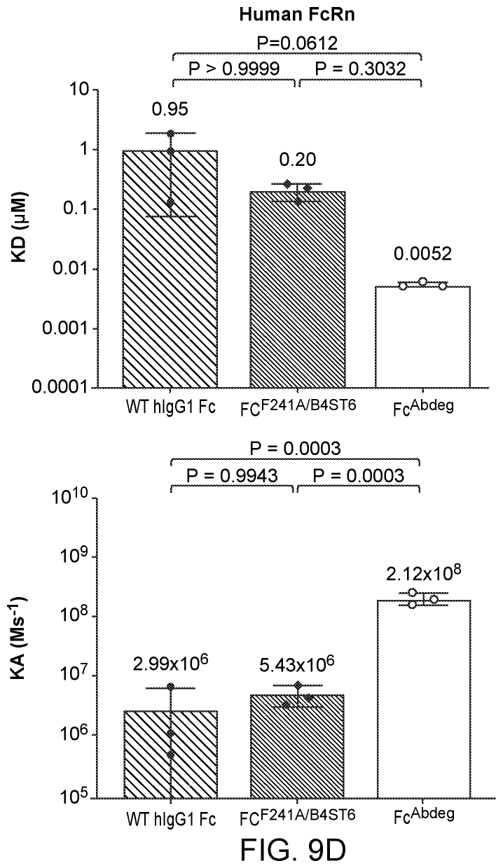

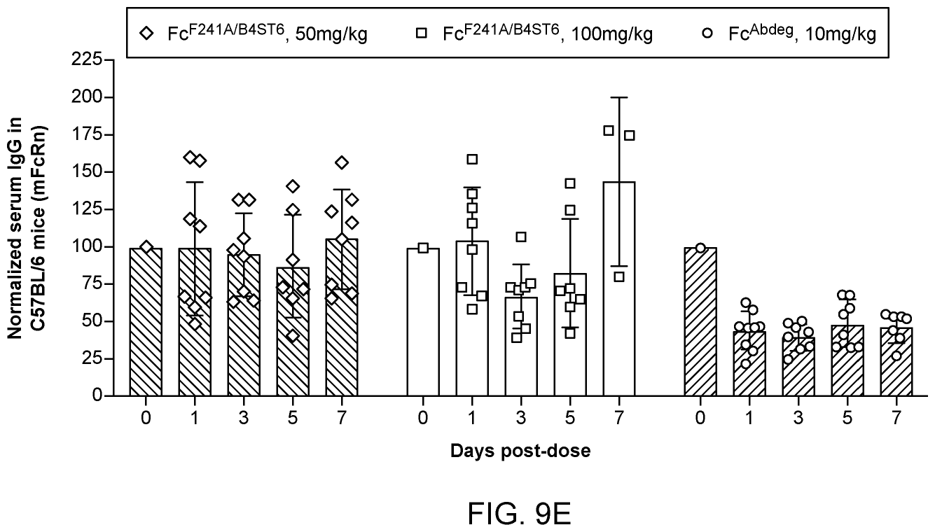

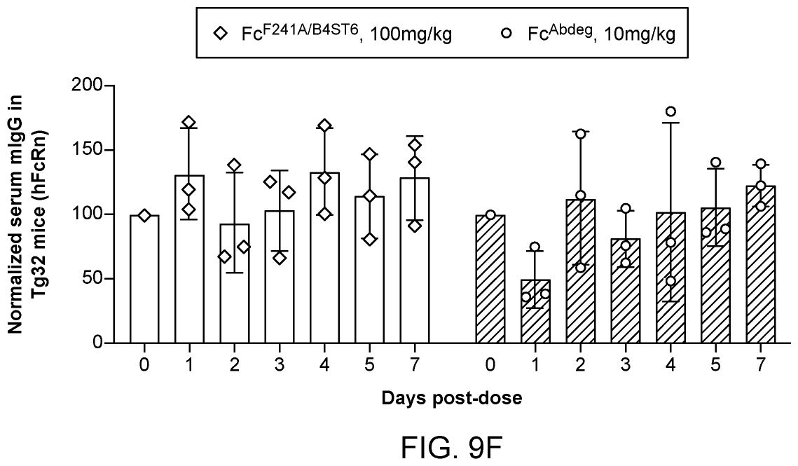

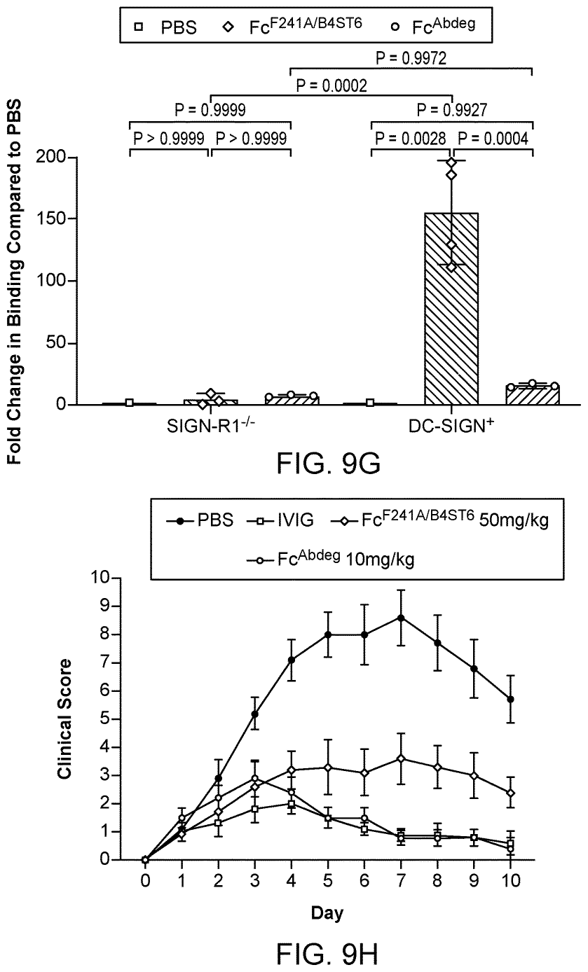

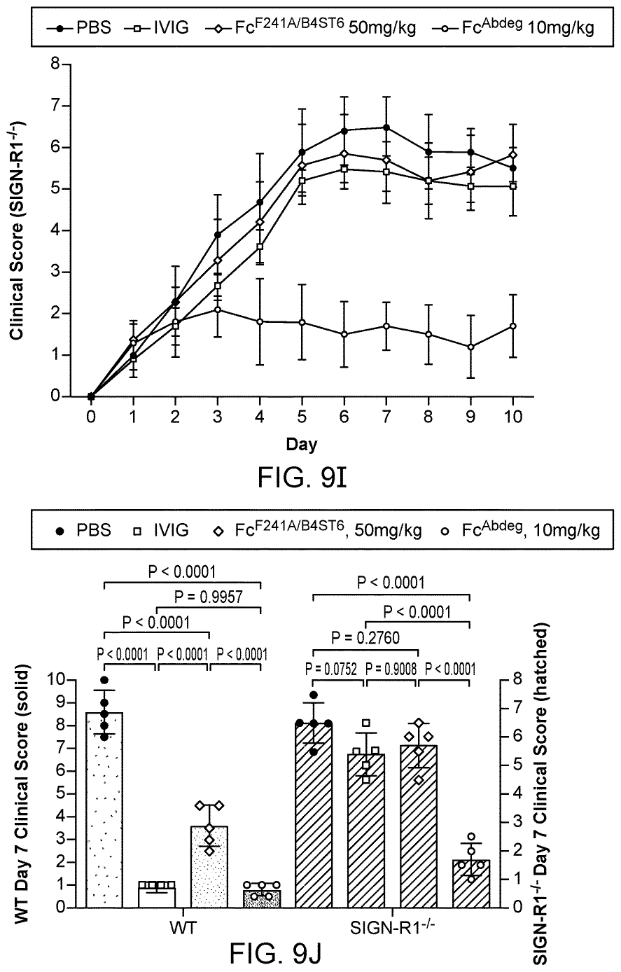

In some embodiments, the one or more additional therapeutic agents is a second modified Fc polypeptide. In some embodiments, the second modified Fc polypeptide is a human IgG1 Fc polypeptide. In some embodiments, the second modified Fc polypeptide comprises one or more (e.g., 1, 2, 3, 4, or 5) mutations selected from the group consisting of M252Y, S254T, T256E, H433K, and N434F (numbered according to the EU index of Kabat; also known as “Abdeg” mutations—see FIG. 9A and FIG. 9B; see also bolded and underlined residues of SEQ ID NO: 5, below). IgG1 Fcs containing Abdeg mutations exhibit enhanced affinity to FcRn, thereby allowing the mutated Fcs to outcompete native IgGs for FcRN binding. As a result, Fc^{Abdeg} polypeptides accelerate the depletion of circulation total IgG by saturating FcRn. In some embodiments, the second modified Fc polypeptide comprises or consists of an amino acid sequence as set forth in SEQ ID NO: 5.

(SEQ ID NO: 5)
DKTHTCPPCPAPELLGGPSVFLFPPKPKDTLYITREPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALKFHYTQKSLSLSPG
*Bolded and underlined amino acid residues
correspond to Abdeg mutations (M252Y, S254T,
T256E, H433K, and N434F).

In some embodiments, the Fc polypeptide of the disclosure is co-administered to a subject (e.g., sequentially or simultaneously) with the second modified Fc polypeptide of SEQ ID NO: 5. In some embodiments, the second modified Fc polypeptide is administered to the subject in an amount of 1-20 mg/kg, e.g., 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg. In some embodiments, the second modified Fc polypeptide is administered to the subject weekly. In some embodiments, the second modified Fc polypeptide is administered to the subject for 1-8 weeks (e.g., 1 week, 2 weeks, 3 weeks, 4 weeks, 5 weeks, 6 weeks, 7 weeks, or 8 weeks).

In some embodiments, the one or more additional therapeutic agents is selected from the group consisting of an anti-inflammatory agent, immunosuppressive agent, analgesic, an anti-rheumatic drug, such as a disease-modifying antirheumatic drug (DMARD), a counterirritant, a platelet-boosting drug, a thrombopoietin receptor (TPOR) agonist, physical therapy, surgery, or any combination thereof. In some embodiments, the anti-inflammatory agent is selected from the group consisting of a corticosteroid, a nonsteroidal anti-inflammatory medication (NSAID), anti-inflammatory antibody or an antigen-binding fragment thereof, anti-inflammatory cytokine, kinase inhibitor, IVIG, or any combination thereof. In some embodiments, the one or more additional therapeutic agents is prednisone, prednisolone, methylprednisolone, methotrexate, hydroxychloroquine, sulfasalazine, leflunomide, cyclophosphamide, azathioprine, or a biologic such as tofacitinib, adalimumab, abatacept, anakinra, kineret, certolizumab, etanercept, golimumab, infliximab, rituximab or tocilizumab. For example, if the disease is RA, the second agent may be one or more of: prednisone, prednisolone and methylprednisolone, methotrexate, hydroxychloroquine, sulfasalazine, leflunomide, cyclophosphamide and azathioprine, tofacitinib, adalimumab, abatacept, anakinra, kineret, certolizumab, etanercept, golimumab, infliximab, rituximab or tocilizumab. In some embodiments, the additional agent is 6-mercaptopurine, 6-thioguanine, abatacept, adalimumab, alemtuzumab, aminosalicylates (5-aminoalicylic acid, sulfasalazine, mesalamine, balsalazide, olsalazine), antibiotics, anti-histamines, anti-TNFα (infliximab, adalimumab, certolizumab pegol, natalizumab) ustekinumab, azathioprine, belimumab, beta interferon, calcineurin inhibitors, certolizumab, corticosteroids (prednisone, methylprednisolone), cromolyn, cyclosporin A, cyclosporine, dimethyl fumarate, etanercept, fingolimod, fumaric acid esters, glatiramer acetate, golimumab, hydroxyurea, IFNγ, IL-11, infliximab, leflunomide, leukotriene receptor antagonist, long-acting beta2 agonist, methotrexate, mitoxantrone, mycophenolate mofetil, natalizumab, NSAIDs, ocrelizumab, pimecrolimus, probiotics, retinoids, rituximab, salicylic acid, short-acting beta2 agonist, sulfasalazine, tacrolimus, teriflunomide, theophylline, tocilizumab, ustekinumab, and vedolizumab.

In some embodiments, a composition of the disclosure is administered alone or in combination with the additional therapeutic agent either simultaneously or sequentially dependent upon the condition to be treated. When two or more compositions are administered, the compositions are, e.g., administered in combination (either sequentially or simultaneously). In some embodiments, the one or more additional therapeutic agents are administered immediately before or after the composition, or 5 minutes, 10 minutes, 15 minutes, 20 minutes, 25 minutes, 30 minutes, 45 minutes, 1 hour, 2 hours, 4 hours, 8 hours, 12 hours, 18 hours, 1 day, 2 days, 3 days, 4 days, one week, two weeks, 1 month, or more before or after administration of the composition. In some embodiments, a composition is administered in a single dose or multiple doses.

### Medicaments

The disclosure provides, in some embodiments, a use of the compositions described herein to make a medicament for treating a condition, disease, or disorder described herein. In some embodiments, medicaments are formulated based on the physical characteristics of the subject needing treatment and are formulated in single or multiple formulations based on the stage of the condition, disease, or disorder. Medicaments are packaged in a suitable package with appropriate labels for the distribution to hospitals and clinics in which the label is for the indication of treating a subject having a disease described herein, in some embodiments. Medicaments are packaged as a single or multiple units, in some embodiments. Instructions for the dosage and administration of the compositions are included with the packages as described below, in some embodiments. The disclosure is further directed to medicaments comprising a composition described herein and a pharmaceutically acceptable carrier, diluent, or excipient. In some embodiments, the pharmaceutically acceptable carrier, diluent, or excipient is selected from the group consisting of a stabilizer, buffer, surfactant, filler, solvent, tonicity or osmolarity adjusting agent, antioxidant, adjuvant, and antimicrobial agent.

### Methods of Manufacture

Disclosed herein, in some embodiments, are methods for manufacturing Fc polypeptides having high levels of sialylation, such as the modified Fc polypeptides disclosed herein. The modified Fc polypeptides of the present invention are, in some embodiments, produced under conditions that result in increased amount of Fc sialylation at the Fc N-glycan (i.e., Asn297) as compared to an unmodified (e.g., native/parent) Fc polypeptide.

### Recombinant Expression System

Disclosed herein, in some embodiments, are methods, systems, and vectors for effectuating the expression of recombinant polypeptides, particularly modified Fc polypeptides of the disclosure, in a host cell.

Disclosed herein, in some embodiments, are recombinant expression systems comprising a nucleic acid expression vector (e.g., a plasmid, RNA vector, virus, viral vector, or other suitable replicon) comprising: (1) a first expression cassette comprising a first mammalian promoter operably linked to a polynucleotide encoding a modified Fc polypeptide having: (i) an amino acid sequence at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) identical to the sequence of SEQ ID NO: 2 and (ii) an aliphatic amino acid residue at position 241 (numbered according to Kabat; corresponding to amino acid residue 32 of SEQ ID NO: 2); and (b) a second expression cassette comprising a second mammalian promoter operably linked to a polynucleotide encoding a beta-galactoside alpha-2,6-sialyltransferase 1 (ST6GAL1) enzyme. In some embodiments, the aliphatic amino acid at position 241 is an Ala (F241A). The second expression cassette further comprises, in some embodiments, a polynucleotide encoding a beta-1,4-galactosyltransferase 1 (B4GALT1) enzyme. In some embodiments, the polynucleotide encoding the B4GALT1 enzyme is operatively linked to the second promoter. In some embodiments, the second expression cassette further comprises an internal ribosome entry site (IRES) sequence positioned between the polynucleotide encoding the ST6GAL1 enzyme and the polynucleotide encoding the B4GALT1 enzyme. In some embodiments, the first promoter and the second promoter are each independently selected from the group consisting of a murine cytomegalovirus (CMV) promoter, elongation factor 1α (EF1α) promoter, eukaryotic elongation factor 2 (EEF2) promoter, glyceraldehyde 3-phosphate dehydrogenase (GAPDH) promoter, phosphoglycerate kinase (PGK) promoter, actin promoter, and ubiquitin promoter.

Disclosed herein, in some embodiments, are recombinant expression systems comprising a nucleic acid expression vector (e.g., a plasmid, RNA vector, virus, viral vector, or other suitable replicon) comprising: (1) a first expression cassette comprising a first mammalian promoter operably linked to a polynucleotide encoding a modified Fc polypeptide having: (i) an amino acid sequence at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) identical to the sequence of SEQ ID NO: 2 and (ii) an aliphatic amino acid residue at position 241 (numbered according to Kabat; corresponding to amino acid residue 32 of SEQ ID NO: 2), a polynucleotide encoding ST6GAL1, and an IRES sequence position therebetween; and (b) a second expression cassette comprising a second mammalian promoter operably linked to a polynucleotide encoding a B4GAL1 enzyme. In some embodiments, the aliphatic amino acid at position 241 is an Ala (F241A). In some embodiments, the first promoter and the second promoter are each independently selected from the group consisting of a murine cytomegalovirus (CMV) promoter, elongation factor 1α (EF1α) promoter, eukaryotic elongation factor 2 (EEF2) promoter, glyceraldehyde 3-phosphate dehydrogenase (GAPDH) promoter, phosphoglycerate kinase (PGK) promoter, actin promoter, and ubiquitin promoter.

Disclosed herein, in some embodiments, are recombinant expression systems comprising a nucleic acid expression vector (e.g., a plasmid, RNA vector, virus, viral vector, or other suitable replicon) comprising: (1) a first expression cassette comprising a first mammalian promoter operably linked to a polynucleotide encoding a modified Fc polypeptide having: (i) an amino acid sequence at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) identical to the sequence of SEQ ID NO: 2 and (ii) an aliphatic amino acid residue at position 241 (numbered according to Kabat; corresponding to amino acid residue 32 of SEQ ID NO: 2), a polynucleotide encoding B4GALT1, and an IRES sequence position therebetween; and (b) a second expression cassette comprising a second mammalian promoter operably linked to a polynucleotide encoding a ST6GAL1 enzyme. In some embodiments, the aliphatic amino acid at position 241 is an Ala (F241A). In some embodiments, the first promoter and the second promoter are each independently selected from the group consisting of a murine cytomegalovirus (CMV) promoter, elongation factor 1α (EF1α) promoter, eukaryotic elongation factor 2 (EEF2) promoter, glyceraldehyde 3-phosphate dehydrogenase (GAPDH) promoter, phosphoglycerate kinase (PGK) promoter, actin promoter, and ubiquitin promoter.

Disclosed herein, in some embodiments, are recombinant expression systems comprising a nucleic acid expression vector (e.g., a plasmid, RNA vector, virus, viral vector, or other suitable replicon) comprising an expression cassette comprising a mammalian promoter operably linked to: (1) a first polynucleotide encoding a modified Fc polypeptide having: (i) an amino acid sequence at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) identical to the sequence of SEQ ID NO: 2 and (ii) an aliphatic amino acid residue at position 241 (numbered according to Kabat; corresponding to amino acid residue 32 of SEQ ID NO: 2); (2) a second polynucleotide encoding an ST6GAL1 enzyme; (3) a first TRES sequence positioned between the first polynucleotide and second polynucleotide; (4) a third polynucleotide encoding a B4GALT1 enzyme; and (5) a second TRES sequence positioned between the second polynucleotide and the third polynucleotide. In some embodiments, the aliphatic amino acid at position 241 is an Ala (F241A). In some embodiments, the promoter is selected from the group consisting of a murine cytomegalovirus (CMV) promoter, elongation factor 1α (EF1α) promoter, eukaryotic elongation factor 2 (EEF2) promoter, glyceraldehyde 3-phosphate dehydrogenase (GAPDH) promoter, phosphoglycerate kinase (PGK) promoter, actin promoter, and ubiquitin promoter.

Disclosed herein, in some embodiments, are recombinant expression systems comprising two or more (e.g., 2, 3, or more) nucleic acid expression vectors (e.g., plasmids, RNA vectors, viruses, viral vectors, or other suitable replicons) that together encode: (a) a modified Fc polypeptide having: (i) an amino acid sequence at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) identical to the sequence of SEQ ID NO: 2 and (ii) an aliphatic amino acid residue at position 241 (numbered according to Kabat; corresponding to amino acid residue 32 of SEQ ID NO: 2); and (b) a ST6GAL1 enzyme; and/or, optionally (c) a B4GALT1 enzyme.

In some embodiments, the two or more (e.g., 2, 3, or more) nucleic acid expression vectors (e.g., plasmids, RNA vectors, viruses, viral vectors, or other suitable replicons) are two expression vectors. In some embodiments, the two expression vectors comprise: (a) a first expression vector comprising an expression cassette comprising first promoter operably linked to a polynucleotide encoding a modified Fc polypeptide having: (i) an amino acid sequence at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) identical to the sequence of SEQ ID NO: 2 and (ii) an aliphatic amino acid residue at position 241 (numbered according to Kabat; corresponding to amino acid residue 32 of SEQ ID NO: 2); and (b) a second expression vector comprising an expression cassette comprising second promoter operably linked to a polynucleotide encoding an ST6GAL1 enzyme; and, optionally (c) a polynucleotide encoding an B4GALT1 enzyme. In some embodiments, the polynucleotide encoding the ST6GAL1 enzyme and the polynucleotide encoding the B4GALT1 enzyme are separated by an TRES sequence.

In some embodiments, the two expression vectors comprise: (a) a first expression vector comprising an expression cassette comprising first promoter operably linked to a polynucleotide encoding a modified Fc polypeptide having: (i) an amino acid sequence at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) identical to the sequence of SEQ ID NO: 2 and (ii) an aliphatic amino acid residue at position 241 (numbered according to Kabat; corresponding to amino acid residue 32 of SEQ ID NO: 2); and a polynucleotide encoding an ST6GAL1 enzyme; and (b) a second expression vector comprising an expression cassette comprising second promoter operably linked to a polynucleotide encoding a B4GALT1 enzyme. In some embodiments, the polynucleotide encoding the modified Fc polypeptide and the polynucleotide encoding the ST6GAL1 enzyme are separated by an TRES sequence.

In some embodiments, the two expression vectors comprise: (a) a first expression vector comprising an expression cassette comprising first promoter operably linked to a polynucleotide encoding a modified Fc polypeptide having: (i) an amino acid sequence at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) identical to the sequence of SEQ ID NO: 2 and (ii) an aliphatic amino acid residue at position 241 (numbered according to Kabat; corresponding to amino acid residue 32 of SEQ ID NO: 2) and a polynucleotide encoding a B4GALT1 enzyme; and (b) a second expression vector comprising an expression cassette comprising second promoter operably linked to a polynucleotide encoding an ST6GAL1 enzyme. In some embodiments, the polynucleotide encoding the modified Fc polypeptide and the polynucleotide encoding the B4GALT1 enzyme are separated by an TRES sequence.

In some embodiments, the two or more (e.g., 2, 3, or more) nucleic acid expression vectors (e.g., plasmids, RNA vectors, viruses, viral vectors, or other suitable replicons) are three expression vectors. In some embodiments, the three expression vectors comprise: (a) a first expression vector comprising an expression cassette comprising a first promoter operably linked to a polynucleotide encoding a modified Fc polypeptide having: (i) an amino acid sequence at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) identical to the sequence of SEQ ID NO: 2 and (ii) an aliphatic amino acid residue at position 241 (numbered according to Kabat; corresponding to amino acid residue 32 of SEQ ID NO: 2); (b) a second expression vector comprising an expression cassette comprising a second promoter operably linked to a polynucleotide encoding an ST6GAL1 enzyme; and (c) a third expression vector comprising an expression cassette comprising a third promoter operably linked to a polynucleotide encoding an B4GALT1 enzyme.

In some embodiments, the polynucleotide encoding an ST6GAL1 enzyme encodes an ST6GAL1 enzyme having an amino acid sequence of SEQ ID NO: 3 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to the amino acid sequence of SEQ ID NO: 3, as shown below.

MIHTNLKKKFSCCVLVFLLFAVICVWKEKKKGSYYDSFKLQTKEFQVLK
SLGKLAMGSDSQSVSSSSTQDPHRGRQTLGSLRGLAKAKPEASFQVWNK
DSSSKNLIPRLQKIWKNYLSMNKYKVSYKGPGPGIKFSAEALRCHLRDH
VNVSMVEVTDFPFNTSEWEGYLPKESIRTKAGPWGRCAVVSSAGSLKSS
QLGREIDDHDAVLRFNGAPTANFQQDVGTKTTIRLMNSQLVTTEKRFLK
DSLYNEGILIVWDPSVYHSDIPKWYQNPDYNFFNNYKTYRKLHPNQPFY
ILKPQMPWELWDILQEISPEEIQPNPPSSGMLGIIIMMTLCDQVDIYEF
LPSKRKTDVCYYYQKFFDSACTMGAYHPLLYEKNLVKHLNQGTDEDIYL
LGKATLPGFRTIHC
(SEQ ID NO: 3; UniProt ID No .: P15907-1)

In some embodiments, the polynucleotide encoding an B4GALT1 enzyme encodes an B4GALT1 enzyme having an amino acid sequence of SEQ ID NO: 4 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to the amino acid sequence of SEQ ID NO: 4, as shown below.

MRLREPLLSGSAAMPGASLQRACRLLVAVCALHLGVTLVYYLAGRDLSR
LPQLVGVSTPLQGGSNSAAAIGQSSGELRTGGARPPPPLGASSQPRPGG
DSSPVVDSGPGPASNLTSVPVPHTTALSLPACPEESPLLVGPMLIEFNM
PVDLELVAKQNPNVKMGGRYAPRDCVSPHKVAIIIPFRNRQEHLKYWLY
YLHPVLQRQQLDYGIYVINQAGDTIFNRAKLLNVGFQEALKDYDYTCFV
FSDVDLIPMNDHNAYRCFSQPRHISVAMDKFGFSLPYVQYFGGVSALSK
QQFLTINGFPNNYWGWGGEDDDIFNRLVFRGMSISRPNAVVGRCRMIRH
SRDKKNEPNPQRFDRIAHTKETMLSDGLNSLTYQVLDVQRYPLYTQITV
DIGTPS
(SEQ ID NO: 4; UniProt ID No .: P15291-1)

In some embodiments, the polynucleotides encoding any one of the polypeptides disclosed herein (e.g., modified Fc polypeptide, ST6GAL1 enzyme, or B4GALT1 enzyme) is codon-optimized. Codon-optimization refers to a process of modifying a nucleic acid sequence in accordance with the principle that the frequency of occurrence of synonymous codons (e.g., codons that code for the same amino acid) in coding DNA is biased in different species. Such codon degeneracy allows an identical polypeptide to be encoded by a variety of nucleotide sequences. Sequences modified in this way are referred to herein as “codon-optimized.” This process may be performed on any of the sequences described in this specification to enhance expression or stability. The sequence surrounding the translational start site can be converted to a consensus Kozak sequence by any suitable methods.

### Expression Vectors

A variety of vectors for the delivery of polynucleotides encoding exogenous proteins to the a host cell have been developed. Expression vectors for use in the compositions and methods described herein may contain one or more (e.g., 1, 2, 3, or more) polynucleotides encoding one or more (e.g., 1, 2, 3, or more) polypeptides of the disclosure, and may further include, for example, one or more (e.g., 1, 2, 3, 4, 5, 6, 7, 8, 9, 10, or more) nucleic acid elements used to regulate the expression of these agents and/or the integration of such polynucleotides into the genome of a host cell.

In some embodiments, the vector is an autonomously replicating vector, i.e., a vector which exists as an extrachromosomal entity, the replication of which is independent of chromosomal replication, e.g., a plasmid, bacteriophage, extrachromosomal element, mini-chromosome, or an artificial chromosome. Alternatively, the vector is one which, when introduced into a host cell, is integrated into the host cell genome and replicated together with the chromosome(s) into which it has been integrated, in some embodiments. Certain vectors that are used for the expression of one or more engineered polypeptides described herein, in some embodiments, include plasmids that contain regulatory sequences, such as promoter and, optionally, enhancer regions, which direct gene transcription. Other useful vectors for expression of one or more polypeptides of the disclosure contain polynucleotide sequences that enhance the rate of translation of these genes or improve the stability or nuclear export of the mRNA that results from gene transcription. These sequence elements include, e.g., 5′ and 3′ untranslated regions, an internal ribosome entry site (IRES), and polyadenylation signal site in order to direct efficient transcription of the gene carried on the expression vector. The expression vectors suitable for use with the compositions and methods described herein may also contain a polynucleotide encoding a marker for selection of cells that contain such a vector. An example of a suitable selection marker is a glutamine synthetase (GS) gene. Additional examples of a suitable marker are genes that encode resistance to antibiotics, such as ampicillin, chloramphenicol, kanamycin, nourseothricin, zeocin, nourseothricin, carbenicillin, tetracycline, streptomycin, and spectinomycin.

In some embodiments, expression vectors of the present disclosure further include a polynucleotide encoding a protein tag, such as, a His-tag (e.g., 6×-His), maltose binding protein tag, SNAP tag, FLAG tag, halotag, fluorescent protein tag, and the like.

### Viral Vectors

Viral genomes provide a rich source of vectors that can be used for the efficient delivery of exogenous genes into a host cell. Viral genomes are particularly useful vectors for gene delivery as the polynucleotides contained within such genomes are typically incorporated into the nuclear genome of a host cell by generalized or specialized transduction. These processes occur as part of the natural viral replication cycle and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, and cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include, but are not limited to, avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus. Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses.

### Regulatory Elements

Recognition and binding of the polynucleotide encoding one or more polypeptides disclosed herein by mammalian RNA polymerase is important for gene expression. As such, one may include sequence elements within the polynucleotide(s) that exhibit a high affinity for transcription factors that recruit RNA polymerase and promote the assembly of the transcription complex at the transcription initiation site. Such sequence elements include, e.g., a mammalian promoter, the sequence of which is recognized and bound by specific transcription initiation factors and ultimately RNA polymerase.

Polynucleotides suitable for use with the compositions and methods described herein also include those that encode a modified Fc polypeptide of the disclosure downstream of a mammalian promoter. In some embodiments, the mammalian promoter is selected from the group consisting of elongation factor 1α (EF1α) promoter, eukaryotic elongation factor 2 (EEF2) promoter, glyceraldehyde 3-phosphate dehydrogenase (GAPDH) promoter, phosphoglycerate kinase (PGK) promoter, actin promoter, and ubiquitin promoter.

Alternatively, promoters derived from viral genomes can also be used for the stable expression of these agents in mammalian cells. Examples of functional viral promoters that can be used to promote mammalian expression of these agents are adenovirus late promoter, vaccinia virus 7.5K promoter, simian virus 40 (SV40) promoter, cytomegalovirus promoter, tk promoter of herpes simplex virus (HSV), mouse mammary tumor virus (MMTV) promoter, long terminal repeat (LTR) promoter of human immunodeficiency virus (HIV), promoter of moloney virus, Epstein barr virus (EBV), Rous sarcoma virus (RSV), and the cytomegalovirus (CMV) promoter (e.g., murine CMV promoter).

Once a polynucleotide encoding one or more recombinant polypeptides of the disclosure has been internalized by the host cell extrachromosomally and/or incorporated into the nuclear DNA of the host cell, the transcription of this polynucleotide can be induced by methods known in the art. For example, expression can be induced by exposing the host cell to an external chemical reagent, such as an agent that modulates the binding of a transcription factor and/or RNA polymerase to the promoter and, thus, regulates gene expression. The chemical reagent can serve to facilitate the binding of RNA polymerase and/or transcription factors to the promoter, e.g., by removing a repressor protein that has bound the promoter. Alternatively, the chemical reagent can serve to enhance the affinity of the promoter for RNA polymerase and/or transcription factors such that the rate of transcription of the gene located downstream of the promoter is increased in the presence of the chemical reagent. Examples of chemical reagents that potentiate polynucleotide transcription by the above mechanisms are tetracycline and doxycycline. These reagents are commercially available (Life Technologies, Carlsbad, CA) and can be administered to a host cell in order to promote gene expression according to established protocols.

Other gene regulatory elements that may be included in polynucleotides for use in the compositions and methods described herein are enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode a modified Fc polypeptide of the disclosure and additionally a mammalian enhancer sequence. Many enhancer sequences are now known from mammalian genes, and examples are enhancers from the genes that encode mammalian globin, elastase, albumin, a-fetoprotein, and insulin. Enhancers for use in the compositions and methods described herein also include those that are derived from the genetic material of a virus capable of infecting a eukaryotic cell. Examples are the SV40 enhancer on the late side of the replication origin (bp 100-270), the CMV early promoter enhancer, the polyoma enhancer on the late side of the replication origin, and adenovirus enhancers. An enhancer may be spliced into a vector containing a polynucleotide encoding a water-forming NADH oxidase, for example, at a position 5′ or 3′ to this gene. In a preferred orientation, the enhancer is positioned at the 5′ side of the promoter, which in turn is located 5′ relative to the polynucleotide encoding a modified Fc polypeptide of the disclosure.

Other exemplary regulatory elements suitable for use with the expression vectors described herein include untranslated regions (UTR, such as a 5′ UTR and/or a 3′ UTR) insulator elements, terminator elements, polyadenylation signals, response elements, and other functional elements or signals.

### Host Cells

The modified Fc polypeptides of the present invention are produced in a host expression system, e.g., host cells (e.g., mammalian cells), capable of expressing the modified Fc polypeptides of the disclosure and/or one or more glycosyltransferase enzymes (e.g., ST6GAL1 and/or B4GALT1), in some embodiments. Typically, such host expression systems may comprise bacterial, fungal, plant, mammalian, or insect expression systems. In some embodiments, the host cell is a mammalian host cell, such as a Chinese hamster ovary (CHO) cell line, (e.g., CHO-K1; ATCC CCL-61), Green Monkey cell line (COS) (e.g., COS 1 (ATCC CRL-1650), COS 7 (ATCC CRL-1651)); mouse cell (e.g., NS/0), Baby hamster kidney (BHK) cell line (e.g. ATCC CRL-1632 or ATCC CCL-10), or human cell (e.g. HEK293 (ATCC CRL-1573) or 293T (ATCC CRL-11268)), or any other suitable cell line available from public depositories such as the American Type Culture Collection, Rockville, Md. Further, an insect cell line, such as a Lepidoptora cell line, e.g., Sf9, a plant cell line, a fungal cell line, e.g., yeast such as, for example, Saccharomyces cerevisiae, Pichia pastoris, Hansenula spp., or a bacterial expression system based on Bacillus, such as B. subtilis, or E. coli can be used. Modifications to host cells may be required to ensure that N-linked glycosylation and glycan maturation occur to result in a complex, biantennary sugar as typically found on the Fc domain of human IgG. In some embodiments, the mammalian host cell described herein is transformed (e.g., transfected or transduced) with any one of the recombinant expression systems described herein.

Despite the availability of several other mammalian cell lines, a majority of recombinant therapeutic proteins produced today are made in CHO cells. Their strengths include, e.g., robust growth as adherent cells or in suspension, adaptability to serum-free and chemically defined media, high productivity, and an established history of regulatory approval for therapeutic recombinant protein production. They are also very amenable to genetic modifications, and the methods used for cell transfection, recombinant protein expression, and clone selection are well-characterized. CHO cells also provide human-compatible post-translational modifications. As used herein, “CHO cells” include, but are not limited to, e.g., CHO-K1, CHO-DG44, CHO-M, CHO-S, CHO GS knockout, and variants and derivatives thereof.

Accordingly, the host cells described herein are modified, in some embodiments, with one or more (e.g., 1, 2, 3, or more) recombinant expression systems of the disclosure to achieve desired levels of recombinant protein expression, including expression of a modified Fc polypeptide (e.g., F241A Fc) and/or one or more (e.g., 1, 2, or more) glycosyltransferase enzymes (e.g., ST6GAL1 and/or B4GALT1). In some embodiments, the host cells described herein are modified such that the ST6GAL1 and B4GALT1 enzyme are both expressed at a desired ratio. For example, the host cell (e.g., mammalian host cell) is modified, in some embodiments, to achieve a ratio of ST6GAL1 and B4GALT1 enzyme expression of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20 (mol:mol). In some embodiments, the ratio of the quantity of ST6GAL1 and B4GALT1 protein expression in the host cell is determined by the strength of the promoter sequence(s) to which each of these proteins are operably linked. In some embodiments, the ratio of the quantity of ST6GAL1 and B4GALT1 protein expression in the host cell is determined by one or more regulatory sequences present in the vector(s) that contain polynucleotide(s) encoding the enzymes (e.g., enhancers, polyadenylation signals, terminators, insulators, UTR sequences, and the like). In some embodiments, the ratio of the quantity of ST6GAL1 and B4GALT1 protein expression in the host cell is determined by the host cell type. In some embodiments, the ratio of the quantity of ST6GAL1 and B4GALT1 protein expression in the host cell is determined by one or more factors intrinsic to the host cell.

Furthermore, the methods described herein may include maintaining viability of host cell at a level sufficient to allow for high levels of sialylation of the modified Fc polypeptides described herein. Without wishing to be bound by any theory, lysis of host cells in culture may result in release of sialidase enzymes into the culture medium, which may reduce the overall levels of Fc sialylation by sialidase-mediated cleavage of SA moieties from the Fc glycan. Accordingly, the present disclosure provides methods for maintaining viability of host cells at or above a predetermined value, such as at least 50%, at least 60%, at least 70%, at least 80%, at least 90% viability after between 10 and 20 days (e.g., 10, 11, 12, 13, 14, 15, 16, 17, 18, 19, or 20 days) in culture. Host cell viability may be maintained and assessed using any suitable method.

### Culture Conditions

Disclosed herein, in certain embodiments, are methods for culturing a host cell of the disclosure in a medium under conditions permitting expression of one or more polypeptides encoded by a polynucleotide disclosed herein, and purifying the one or more polypeptides from the cultured cell or the medium of the cell. To increase levels of sialylation of the modified Fc polypeptides described herein, culture conditions under which the host cells of the disclosure are maintained can be optimized. For example, to increase the SA content, production rate is decreased and osmolality is generally maintained within a lower margin suitable for the particular host cell being cultured, in some embodiments. Osmolality in the range from about 250 mOsm to about 450 mOsm (e.g., between 250-260, 260-270, 270-280, 280-290, 290-300, 300-310, 310-320, 320-330, 330-340, 340-350, 350-360, 360-370, 370-380, 380-390, 390-400, 400-410, 410-420, 420-430, 430-440, and 440-450 mOsm) is appropriate for increased SA content, in some embodiments. Previous studies have reported that the content of SA in antibody-linked sugar side chains differs significantly if antibodies were produced as ascites or in serum-free or serum-containing culture media. Moreover, others have shown that use of different bioreactors for cell growth and oxygenation of the medium influenced the amount of galactose and SA in antibody-linked glycans.

Furthermore, culture conditions are further modified to increase the rate of sialylation of polypeptides of the disclosure, in some embodiments. One method for enhancing Fc sialylation is by adding agents into the culture medium that enhance sialylation (e.g., by inhibiting sialidase activity), such as, e.g., uridine, manganese, copper, dexamethasone, hydrocortisone, N-acetylmannoseamine, tetraacetylated ManNAc, N-azidoacetyl D-mannosamine, 1,3,4-O-Bu₃ManNAc, α(2,3)-Dehydro-2-deoxy-N-acetylneuraminic acid (DANA), siastatin B, fetuin, and glycerol.

In some embodiments, Fc sialylation is enhanced by modifying the culture conditions to a pH that is conducive to sialylation, such as a pH of about 7.2 (e.g., 7.0, 7.1, 7.2, 7.3, or 7.4).

### Methods for Delivery of Recombinant Nucleic Acids to Host Cells

Vectors disclosed herein can be introduced into a cell by a variety of methods, including transformation, transfection, direct uptake, projectile bombardment, and by encapsulation of the vector in a liposome. Examples of suitable methods of transfecting or transforming cells are calcium phosphate precipitation, electroporation, microinjection, infection, lipofection, and direct uptake. Genes encoding polypeptides of the disclosure can also be introduced into host cells by targeting a vector containing a gene encoding such an agent to cell membrane phospholipids.

For example, electroporation can be used to permeabilize host cells by the application of an electrostatic potential to the cell of interest. Host cells subjected to an external electric field in this manner are subsequently predisposed to the uptake of exogenous nucleic acids. A similar technique, nucleofection, utilizes an applied electric field in order to stimulate the uptake of exogenous polynucleotides into the nucleus of a eukaryotic cell.

Additional techniques useful for the transfection of target cells are the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a target cell.

Lipofection represents another technique useful for transfection of target cells. This method involves the loading of nucleic acids into a liposome, which often presents cationic functional groups, such as quaternary or protonated amines, towards the liposome exterior. This leads to uptake of the exogenous nucleic acids, for example, by direct fusion of the liposome with the cell membrane or by endocytosis of the complex. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers, polyethylenimine, and diethylaminoethyl (DEAE)-dextran. Magnetic beads are another tool that can be used to transfect target cells in a mild and efficient manner, as this methodology utilizes an applied magnetic field in order to direct the uptake of nucleic acids.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s).

Magnetofection can also be used to deliver nucleic acids to target cells. The magnetofection principle is to associate nucleic acids with cationic magnetic nanoparticles. The magnetic nanoparticles are made of iron oxide, which is fully biodegradable, and coated with specific cationic proprietary molecules varying upon the applications. Their association with the nucleic acid vectors is achieved by salt-induced colloidal aggregation and electrostatic interaction. The magnetic particles are then concentrated on the target cells by the influence of an external magnetic field generated by magnets.

Another useful tool for inducing the uptake of exogenous nucleic acids by target cells is sonoporation, a technique that involves the use of sound (typically ultrasonic frequencies) for modifying the permeability of the cell plasma membrane to permeabilize the cells and allow polynucleotides to penetrate the cell membrane.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence.

### Protein Purification

A modified Fc polypeptide of the disclosure can be recovered and purified from recombinant cell cultures by any suitable methods, including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. High performance liquid chromatography (HPLC) can also be employed for purification.

Fc polypeptides of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the Fc polypeptides of the present disclosure are glycosylated (e.g., sialylated).

### Analytical Methods for Assessing Fc Sialylation

Fc sialylation can be assessed by any suitable assay. For example, sialylation of Fc polypeptides is assessed by way of HPLC. In some embodiments, sialylation of the modified Fc polypeptides of the disclosure is assessed by way of hydrophilic interaction liquid chromatography (HILIC). In some embodiments, sialylation of the modified Fc polypeptides of the disclosure is assessed by way of MS. In some embodiments, sialylation of the modified Fc polypeptides of the disclosure is assessed by way of size exclusion chromatography (SEC). In some embodiments, sialylation of the modified Fc polypeptides of the disclosure is assessed by way of HPLC, HILIC, MS, SEC, HPLC-UV, or any combination thereof.

### Additional Fc Modifications

The disclosed Fc polypeptides are further modified (i.e., in addition to an aliphatic amino acid substitution at amino acid residue 241 of the Fc heavy chain, such as an F241A substitution), in some embodiments, using techniques known in the art for various purposes. In some embodiments, Fc polypeptides of the disclosure are further modified to increase half-life of the antigen-binding fragment in circulation when administered to a subject (e.g., a human). In some embodiments, an Fc region disclosed herein is modified to reduce or silence effector functions of the Fc polypeptide (e.g., antibody-dependent cellular cytotoxicity (ADCC), complement dependent cytotoxicity (CDC), opsonization, phagocytosis, transcytosis, neutralization of infectivity, inflammation, mucosal immunity, and neonatal immunity). In some embodiments, the modified Fc polypeptides of the disclosure are further modified to modulate binding of the Fc polypeptide to one or more Fc receptors.

Covalent modifications of an Fc polypeptide are also included herein. In some embodiments, covalent modifications are made by chemical synthesis or by enzymatic or chemical cleavage of the polypeptide, if applicable. In some embodiments, other types of covalent modifications are introduced by reacting targeted amino acid residues with an organic derivatizing agent that is capable of reacting with selected side chains or the N- or C-terminal residues.

Cysteinyl residues are most commonly reacted with alpha-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, alpha-bromo-beta-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

In some embodiments, histidyl residues are derivatized by reaction with diethylpyrocarbonate at pH 5.5-7.0 because this agent is relatively specific for the histidyl side chain. In some embodiments, para-bromophenacyl bromide also is useful; the reaction, in some embodiments, is performed in 0.1 M sodium cacodylate at pH 6.0.

In some embodiments, lysinyl and amino-terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

In some embodiments, arginyl residues are modified by reaction with one or several conventional reagents, such as phenylglyoxal, α(2,3)-butanedione, 1,2-cyclohexanedione, and ninhydrin. Derivatization of arginine residues requires that the reaction be performed in alkaline conditions because of the high pKa of the guanidine functional group. Furthermore, these reagents, in some embodiments, react with the groups of lysine as well as the arginine epsilon-amino group.

In some embodiments, the specific modification of tyrosyl residues is made, with particular interest in introducing spectral labels into tyrosyl residues by reaction with aromatic diazonium compounds or tetranitromethane. Most commonly, N-acetylimidazole and tetranitromethane are used to form O-acetyl tyrosyl species and 3-nitro derivatives, respectively, in some embodiments. Tyrosyl residues are iodinated using ¹²⁵I or ¹³¹I to prepare labeled proteins for use in radioimmunoassay.

Carboxyl side groups (aspartyl or glutamyl) are specifically modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl)carbodiimide. Furthermore, aspartyl and glutamyl residues are converted to asparaginyl and glutaminyl residues by reaction with ammonium ions.

In some embodiments, glutaminyl and asparaginyl residues are deamidated to the corresponding glutamyl and aspartyl residues, respectively. These residues are deamidated under neutral or basic conditions.

Other modifications include hydroxylation of proline and lysine, phosphorylation of hydroxyl groups of seryl or threonyl residues, methylation of the alpha-amino groups of lysine, arginine, and histidine side chains, acetylation of the N-terminal amine, and amidation of any C-terminal carboxyl group.

Another type of covalent modification involves chemically or enzymatically coupling glycosides to the therapeutic agent of the disclosure. These procedures do not require production of the Fc polypeptide in a host cell that has glycosylation capabilities for N- or O-linked glycosylation. Depending on the coupling mode used, in some embodiments, the sugar(s) are attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine.

Another type of covalent modification comprises linking a polypeptide to one of a variety of nonproteinaceous polymers, e.g., polyethylene glycol, polypropylene glycol, polyoxyethylated polyols, polyoxyethylated sorbitol, polyoxyethylated glucose, polyoxyethylated glycerol, polyoxyalkylenes, or polysaccharide polymers such as dextran. In some embodiments, Fc polypeptides of the disclosure are modified by addition of polyethylene glycol (PEG). In some embodiments, PEG modification (PEGylation) leads to one or more of improved circulation time, improved solubility, improved resistance to proteolysis, reduced antigenicity and immunogenicity, improved bioavailability, reduced toxicity, improved stability, and easier formulation.

Fatty acids and fatty acid esters are also suitable moieties for covalently modifying Fc polypeptides of the disclosure and can be saturated or contain one or more units of unsaturation. Fatty acids that are suitable for modifying Fc polypeptides disclosed herein include, e.g., n-dodecanoate (C₁₂, laurate), n-tetradecanoate (C₁₄, myristate), n-octadecanoate (Cis, stearate), n-eicosanoate (C₂₀, arachidate), n-docosanoate (C₂₂, behenate), n-triacontanoate (C₃₀), n-tetracontanoate (C₄₀), cis-Δ9-octadecanoate (C₁₈, oleate), all cis-Δ5,8,11,14-eicosatetraenoate (C₂₀, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

In some embodiments, an Fc polypeptide provided herein is conjugated or linked to a therapeutic moiety, an imaging or detectable moiety, or an affinity tag. Conjugating or linking polypeptides may be performed using any suitable methods. Associations (binding) between compounds and labels include any suitable means, including, but not limited to, covalent and non-covalent interactions, chemical conjugation, as well as recombinant techniques. An Fc polypeptide is conjugated to, or recombinantly engineered with, an affinity tag (e.g., a purification tag), in some embodiments. Affinity tags such as, e.g., poly-histidine tags (e.g., His6) are suitable for use with the compositions and methods described herein.

### Pharmaceutical Compositions

Disclosed herein, in some embodiments, are pharmaceutical compositions comprising: (a) a population of modified Fc polypeptides, each modified Fc polypeptide having (i) an amino acid sequence at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) identical to the sequence of SEQ ID NO: 2 and (ii) an aliphatic amino acid residue (e.g., Ala) at position 241 (numbered according to Kabat; corresponding to amino acid residue 32 of SEQ ID NO: 2), comprising: at least 60% (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the modified Fc polypeptide having a sialic acid (SA) moiety attached to an N-glycan of the Fc polypeptide via an α(2,6) linkage; and (b) a pharmaceutically acceptable carrier, diluent, or excipient. Such compositions are useful for in vitro or in vivo analysis or, in the case of pharmaceutical compositions, for administration to a subject in vivo or ex vivo for treating a subject having a disease or disorder (e.g., inflammatory disease or disorder, such as an autoimmune disease or disorder) with the disclosed polypeptides.

In some embodiments, the carrier, diluent, or excipient is a stabilizer, buffer, surfactant, filler, solvent, tonicity or osmolarity adjusting agent, antioxidant, adjuvant, and antimicrobial agent or other suitable materials. Such materials should be non-toxic and should not interfere with the efficacy of the active ingredient. The precise nature of the carrier or other material will depend on the route of administration.

Pharmaceutical formulations comprising an Fc polypeptide, identified by the methods described herein are prepared for storage by mixing the protein having the desired degree of purity with optional physiologically acceptable carrier, diluent, or excipient, in the form of lyophilized formulations or aqueous solutions, in some embodiments. Acceptable carriers, diluents, or excipients are those that are non-toxic to recipients at the dosages and concentrations employed, and include buffers such as acetate, phosphate, citrate, histidine, TRIS, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as proline, glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose, or sorbitol; salt-forming counter-ions such as sodium; metal complexes (e.g., Zn-protein complexes); and/or non-ionic surfactants such as TWEEN®, PLURONICS®, polyethylene glycol (PEG), polysorbate (e.g., polysorbate 20, 40, 60, and 80), and poloxamer (e.g., P101, P105, P108, P122, P123, P124, P181, P182, P183, P184, P185, P188, P212, P215, P217, P231, P234, P235, P237, P238, P282, P284, P288, P331, P333, P334, P335, P338, P401, P402, P403, and P407). In some embodiments, the pharmaceutical composition is stable as a liquid solution at room temperature.

Acceptable carriers are physiologically acceptable to the administered subject and retain the therapeutic properties of the compounds with/in which it is administered. Acceptable carriers and their formulations are and generally described in, e.g., Remington's Pharmaceutical Sciences, supra. One exemplary carrier is physiological saline. The phrase “pharmaceutically acceptable carrier” as used herein means a pharmaceutically acceptable material, composition or vehicle, such as a liquid or solid filler, diluent, excipient, solvent, or encapsulating material, involved in carrying or transporting the subject compounds from the administration site of one organ, or portion of the body, to another organ, or portion of the body, or in an in vitro assay system. Each carrier is acceptable in the sense of being compatible with the other ingredients of the formulation and not injurious to a subject to whom it is administered. Nor should an acceptable carrier alter the specific activity of the subject compounds.

In some embodiments, a pharmaceutical composition disclosed herein further comprises an acceptable additive to improve the stability of the compounds in composition and/or to control the release rate of the composition. Acceptable additives do not alter the specific activity of the subject compounds. Exemplary acceptable additives include, but are not limited to, a sugar such as mannitol, sorbitol, glucose, xylitol, trehalose, sorbose, sucrose, galactose, dextran, dextrose, fructose, lactose, and mixtures thereof. Acceptable additives are combined with acceptable carriers and/or excipients, such as dextrose, in some embodiments. Alternatively, exemplary acceptable additives include, but are not limited to a surfactant, such as polysorbate 20 or polysorbate 80, to increase stability of the polypeptide and decrease gelling of the solution. In some embodiments, the surfactant is added to the composition in an amount of 0.01% to 5% of the solution. Addition of such acceptable additives increases the stability and half-life of the composition in storage.

In some embodiments, a pharmaceutical composition disclosed herein contains an isotonic buffer such as a phosphate, acetate, histidine, or TRIS buffer in combination with a tonicity agent such as a polyol, sorbitol, sucrose, or sodium chloride, which tonicifies and stabilizes. In some embodiments, a tonicity agent is present in the composition in an amount of about 5%.

In some embodiments, a pharmaceutical composition disclosed herein includes a surfactant such as to prevent aggregation and for stabilization at 0.01 to 0.02% w/v.

In some embodiments, the pH of a pharmaceutical composition disclosed herein ranges from 4.5-6.5 or 4.5-5.5.

In some embodiments, a pharmaceutical composition disclosed herein also contains more than one active compound as necessary for the indication being treated, such as those with complementary activities that do not adversely affect each other. Such molecules are suitably present in combination in amounts that are effective for the purpose intended.

In some embodiments, active ingredients are entrapped in microcapsule prepared, e.g., by coacervation techniques or by interfacial polymerization, e.g., hydroxy methylcellulose or gelatin-microcapsule and poly-(methylmethacrylate) microcapsule, respectively, in colloidal drug delivery systems (e.g., liposomes, albumin microspheres, microemulsions, nanoparticles, and nanocapsules) or in macroemulsions.

Suspensions and crystal forms of polypeptides are also contemplated herein; any suitable methods to may be used to make suspensions and crystal forms.

In some embodiments, a pharmaceutical composition disclosed herein is sterile. In some embodiments, a pharmaceutical composition disclosed herein is sterilized by conventional, any suitable sterilization techniques. For example, sterilization is readily accomplished by filtration through sterile filtration membranes. In some embodiments, the resulting solution is packaged for use or filtered under aseptic conditions and lyophilized, the lyophilized preparation being combined with a sterile solution prior to administration.

Freeze-drying is employed to stabilize polypeptides for long-term storage, such as when a polypeptide is relatively unstable in liquid compositions, in some embodiments.

In some embodiments, excipients such as, e.g., polyols (including mannitol, sorbitol, and glycerol), sugars (including glucose and sucrose), and amino acids (including alanine, glycine, and glutamic acid) act as stabilizers for freeze-dried products. Polyols and sugars are also used to protect polypeptides from freezing and drying-induced damage and to enhance the stability during storage in the dried state, in some embodiments. Sugars are, in some embodiments, effective in both the freeze-drying process and during storage. Other classes of molecules, including mono- and disaccharides and polymers, such as PVP, have also been reported as stabilizers of lyophilized products.

For injection, in some embodiments, a pharmaceutical composition disclosed herein is a powder suitable for reconstitution with an appropriate solution as described above. Examples of these include, but are not limited to, freeze dried, rotary dried, or spray dried powders, amorphous powders, granules, precipitates, or particulates. For injection, the compositions optionally contain stabilizers, pH modifiers, surfactants, bioavailability modifiers, and combinations of these.

Sustained-release preparations are prepared, in some embodiments. Suitable examples of sustained-release preparations include semipermeable matrices of solid hydrophobic polymers containing the polypeptide, which matrices are in the form of shaped articles, e.g., films, or microcapsule. Examples of sustained-release matrices include polyesters, hydrogels (e.g., poly(2-hydroxyethyl-methacrylate), or poly(vinylalcohol)), polylactides, copolymers of L-glutamic acid and y ethyl-L-glutamate, non-degradable ethylene-vinyl acetate, degradable lactic acid-glycolic acid copolymers such as injectable microspheres composed of lactic acid-glycolic acid copolymer and leuprolide acetate, and poly-D-(−)-3-hydroxybutyric acid. While polymers such as ethylene-vinyl acetate and lactic acid-glycolic acid enable release of molecules for over 100 days, certain hydrogels release proteins for shorter time periods. While encapsulated polypeptides remain in the body for a long time, they denature or aggregate as a result of exposure to moisture at 37° C., resulting in a loss of biological activity and possible changes in immunogenicity. Rational strategies devised for stabilization are, in some cases, dependent on the mechanism involved. For example, if the aggregation mechanism is discovered to be intermolecular S— bond formation through thio-disulfide interchange, stabilization is achieved, in some cases, by modifying sulfhydryl residues, lyophilizing from acidic solutions, controlling moisture content, using appropriate additives, and developing specific polymer matrix compositions.

In some embodiments, a pharmaceutical composition disclosed herein is designed to be short-acting, fast-releasing, long-acting, or sustained-releasing, as described herein. In some embodiments, a pharmaceutical composition disclosed herein is formulated for controlled release or for slow release.

The pharmaceutical composition is administered, e.g., by injection, including, but not limited to, subcutaneous, intravitreal, intradermal, intravenous, intra-arterial, intraperitoneal, intracerebrospinal, intraosseous, or intramuscular injection. Excipients and carriers for use in formulation of compositions for each type of injection are contemplated herein. The following descriptions are by example only and are not meant to limit the scope of the compositions. Compositions for injection include, but are not limited to, aqueous solutions (where water soluble) or dispersions, as well as sterile powders for the extemporaneous preparation of sterile injectable solutions or dispersion. For intravenous administration, suitable carriers include physiological saline, bacteriostatic water, or phosphate buffered saline (PBS). In some embodiments, the carrier is a solvent or dispersion medium containing, e.g., water, ethanol, polyol (e.g., glycerol, propylene glycol, and liquid polyethylene glycol, and the like), and suitable mixtures thereof. Fluidity is maintained, e.g., by the use of a coating such as lecithin, by the maintenance of the required particle size in the case of dispersion and by the use of surfactants. Antibacterial and antifungal agents include, e.g., parabens, chlorobutanol, phenol, ascorbic acid, and thimerosal. Isotonic agents, e.g., sugars, polyalcohols such as mannitol, sorbitol, and sodium chloride are included in the composition, in some embodiments. The resulting solutions are packaged for use as is, or lyophilized; the lyophilized preparation is later combined with a sterile solution prior to administration, in some embodiments. For intravenous injection or injection at the site of affliction, the active ingredient will be in the form of a parenterally acceptable aqueous solution which is pyrogen-free and has suitable pH, isotonicity, and stability. Any suitable solutions using, e.g., isotonic vehicles such as Sodium Chloride Injection, Ringer's Injection, and Lactated Ringer's Injection. Preservatives, stabilizers, buffers, antioxidants, and/or other additives are included as needed, in some embodiments. Sterile injectable solutions are prepared by incorporating an active ingredient in the required amount in an appropriate solvent with one or a combination of ingredients enumerated above, as required, followed by filtered sterilization, in some embodiments. Generally, dispersions are prepared by incorporating the active ingredient into a sterile vehicle which contains a basic dispersion medium and the required other ingredients from those enumerated above. In the case of sterile powders for the preparation of sterile injectable solutions, the preferred methods of preparation are vacuum drying and freeze drying which yields a powder of the active ingredient plus any additional desired ingredient from a previously sterile-filtered solution thereof.

Compositions are administered intravenously, in some embodiments, such as by injection of a unit dose. For injection, in some embodiments, an active ingredient is in the form of a parenterally acceptable aqueous solution which is substantially pyrogen-free and has suitable pH, isotonicity, and stability. In some embodiments, one prepares suitable solutions using, e.g., isotonic vehicles such as Sodium Chloride Injection, Ringer's Injection, Lactated Ringer's Injection. Preservatives, stabilizers, buffers, antioxidants, and/or other additives are included, as required, in some embodiments. Additionally, compositions are administered via aerosolization, in some embodiments.

For parenteral administration, the polypeptides are formulated in a unit dosage injectable form (e.g., solution, suspension, or emulsion) in association with a pharmaceutically acceptable, parenteral vehicle. Examples of such vehicles are water, saline, Ringer's solution, dextrose solution, and 5% human serum albumin. Nonaqueous vehicles such as fixed oils and ethyl oleate are also used. In some embodiments, liposomes are used as carriers. The vehicle contains minor amounts of additives such as substances that enhance isotonicity and chemical stability, e.g., buffers and preservatives. The polypeptides are typically formulated in such vehicles at concentrations of about 1 mg/mL to 10 mg/mL.

In some embodiments, a pharmaceutical composition disclosed herein is lyophilized, e.g., to increase shelf-life in storage. When the compositions are considered for use in medicaments or any of the methods provided herein, in some embodiments, it is contemplated that the composition are substantially free of pyrogens such that the composition will not cause an inflammatory reaction or an unsafe allergic reaction when administered to a human subject.

In some embodiments, acceptable carriers contain a compound that stabilizes, increases, or delays absorption or clearance. Such compounds include, e.g., carbohydrates, such as glucose, sucrose, or dextrans; low molecular weight proteins; compositions that reduce the clearance or hydrolysis of peptides; or excipients or other stabilizers and/or buffers. Agents that delay absorption include, e.g., aluminum monostearate and gelatin. In some embodiments, detergents also be used to stabilize or to increase or decrease the absorption of the pharmaceutical composition, including liposomal carriers. To protect from digestion, the compound is, in some embodiments, complexed with a composition to render it resistant to acidic and enzymatic hydrolysis, or the compound is complexed in an appropriately resistant carrier such as a liposome. Protecting compounds from digestion may be achieved using any suitable methods.

### Packages, Kits, and Pre-Filled Containers

Also provided herein are kits containing one or more therapeutic agents described above. The kit includes, in some embodiments, a modified and highly sialylated Fc polypeptide of the disclosure or a composition containing the same in suitable container means.

In some embodiments, a container means comprising a composition described herein is provided. In some embodiments, the container means is any suitable container which houses, e.g., a liquid or lyophilized composition including, but not limited to, a vial, syringe, bottle, and an intravenous (IV) bag or ampoule. A syringe holds any volume of liquid suitable for injection into a subject, including, but not limited to, 0.5 cc, 1 cc, 2 cc, 5 cc, 10 cc, or more.

Provided herein are kits comprising a composition or compositions described herein. In some embodiments, provided herein is a kit for treating a subject having an inflammatory disease or disorder containing an a composition described herein and, optionally, an additional therapeutic agent.

In some embodiments, provided herein is a kit for treating an inflammatory disease or disorder containing a composition described herein and a label attached to or packaged with the container, the label describing use of the composition, optionally, in combination with an additional therapeutic agent.

In some embodiments, the container means of the kits will generally include at least one vial, test tube, flask, bottle, ampoule, syringe, an intravenous (IV) bag, and/or other container means, into which at least one Fc polypeptide of the disclosure or composition containing the same is placed, and/or suitably aliquoted.

The kits, in some embodiments, include a means for containing at least one Fc polypeptide and or composition containing the same in close confinement for commercial sale. In some embodiments, such containers include injection and/or blow-molded plastic containers into which the desired vials are retained. In some embodiments, kits also include printed material for use of the materials in the kit.

Packages and kits additionally include a buffering agent, a preservative, and/or a stabilizing agent in a pharmaceutical formulation, in some embodiments. In some embodiments, each component of the kit is enclosed within an individual container and all of the various containers are within a single package. In some embodiments, disclosure kits are designed for cold storage or room temperature storage.

Additionally, in some embodiments, the preparations contain stabilizers to increase the shelf-life of the kits and include, e.g., bovine serum albumin (BSA). Where the compositions are lyophilized, the kit contains, in some embodiments, further preparations of solutions to reconstitute the lyophilized preparations. Acceptable reconstitution solutions include, e.g., pharmaceutically acceptable phosphate buffered saline (PBS).

In some embodiments, packages and kits further include one or more components for an assay, such as, e.g., an ELISA assay, an HPLC assay, or a mass spectrometry assay. Samples to be tested in this application include, e.g., blood, plasma, serum, tissue sections and secretions, urine, lymph, and products thereof. In some embodiments, packages and kits further include one or more components for collection of a sample (e.g., a syringe, a cup, a swab, etc.).

In some embodiments, packages and kits further include a label specifying information required by US FDA or similar regulatory authority, e.g., a product description, amount and mode of administration, and/or indication of treatment. In some embodiments, packages provided herein include any of the compositions as described herein.

The term “packaging material” refers to a physical structure housing the components of the kit. In some embodiments, the packaging material maintains the components sterile and is made of material commonly used for such purposes (e.g., paper, corrugated fiber, glass, plastic, foil, ampules, etc.). In some embodiments, the label or packaging insert includes appropriate written instructions (e.g., instructing the user of the kit to perform one or more methods disclosed herein). Kits, in some embodiments, additionally include labels or instructions for using the kit components in any method of the disclosure. In some embodiments, a kit includes a compound in a pack or dispenser together with instructions for administering the compound in a method described herein.

In still further embodiments, a kit further comprises a container means for one or more additional therapeutics for an inflammatory disease or disorder.

In some embodiments, instructions include instructions for practicing any of the methods described herein including treatment methods. In some embodiments, instructions additionally include indications of a satisfactory clinical endpoint or any adverse symptoms that occur, or additional information required by regulatory agencies such as the Food and Drug Administration for use on a human subject.

The instructions are, in some embodiments, on “printed matter,” e.g., on paper or cardboard within or affixed to the kit, or on a label affixed to the kit or packaging material, or attached to a vial or tube containing a component of the kit. Instructions are additionally included on a computer readable medium, such as, e.g., CD-ROMs, DVDs, flash memory devices, solid state memory, magnetic disks and disk devices, magnetic tapes, cloud computing systems and services, and the like, in some embodiments. In some cases, the program and instructions are permanently, substantially permanently, semi-permanently, or non-transitorily encoded on the media.

### EXAMPLES

The following examples are put forth to provide those of ordinary skill in the art with a description of how the compositions and methods described herein may be used, made, and evaluated, and are intended to be purely exemplary of the disclosure and are not intended to limit the scope of what the inventors regard as their invention.

### Example 1: F241A Fc Mutant Exhibits Increased Levels of α(2,3) Sialylation as Compared to the Wild-Type Fc Domain

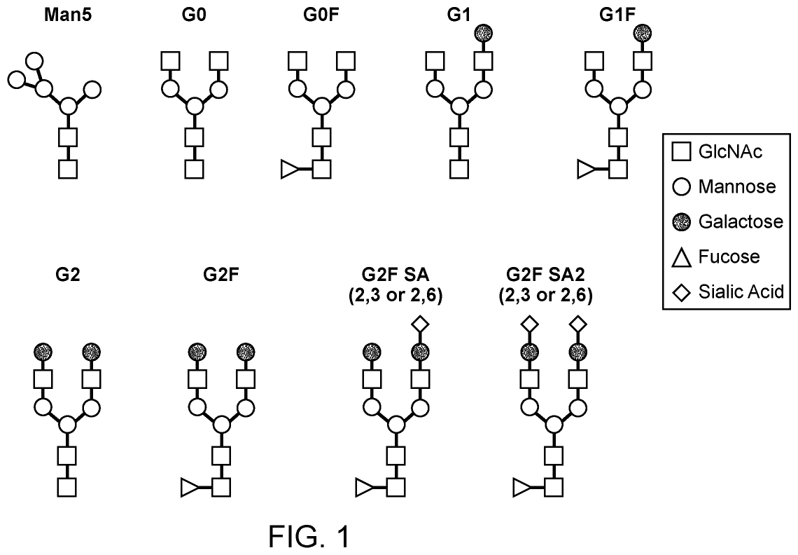

Human IgG1 wild-type (WT) Fc domain or Fc domain bearing the F241A mutation were transiently expressed in Chinese hamster ovary (CHO) cells. Asparagine 297 (Asn297) is the site of a biantennary glycan, which, under normal physiological conditions, is post-translationally modified to manipulate the level of sialic acid linkage. These recombinant Fc domains were purified by size exclusion chromatography (SEC) and suspended in phosphate buffered saline (PBS). N-glycan profiling was performed by high-performance liquid chromatography (HPLC) followed by mass spectrometry (MS) using known standards to define the structure of the biantennary sugar molecule attached to Asn297. N-glycan structures identified using this analysis are exemplified in FIG. 1.

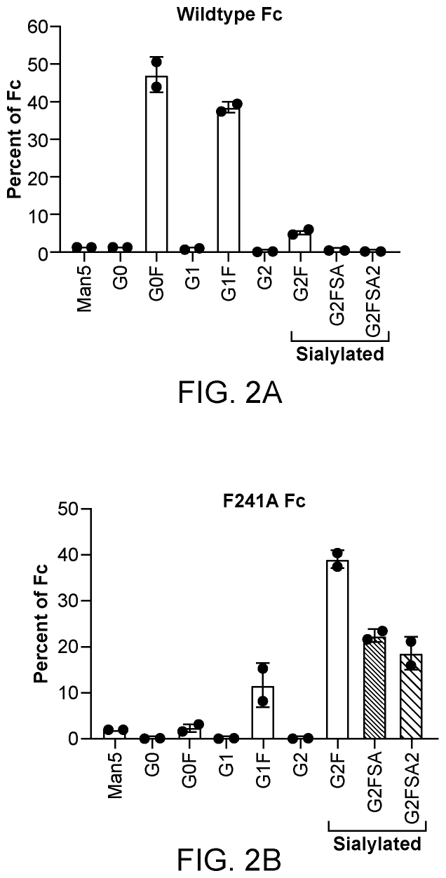

The percentage of Fc polypeptides bearing defined glycan structures at Asn297 is depicted in FIGS. 2A-2B. WT Fc polypeptides predominantly contain N-glycans terminating in N-Acetylglucosamine (GlcNAc; 49%) or in GlcNAc moieties containing a single galactose (G1F; 39%), but little to no SA-containing glycoforms (<1.0%). By comparison, introduction of the F241A mutation in the Fc polypeptides results in a substantial enhancement in the addition of galactose, with 22% of the polypeptides containing mono-2,3 SA (G2FSA) and 19% of molecules containing di-2,3 SA (G2FSA2).

### Example 2: High Levels of α(2,6) Sialylation of the F241A Fc Mutant in a Recombinant Expression System

Human IgG1 Fc domain bearing a phenylalanine (F) to alanine (A) substitution at amino acid position 241 (F241A) was stably expressed in CHO cells as both 10 mL and 1 L cultures. To manipulate the N-glycan structure present on Asn297, the Fc-F241A was expressed alone (Pool 1), expressed with a beta-galactoside alpha-2,6-sialyltransferase 1 (ST6GAL1) enzyme (Pool 2), expressed with ST6GAL1 and a beta-1,4-galactosyltransferase 1 (B4GALT1) enzyme (Pool 3), or expressed with an siRNA targeting the sialic acid transporter, solute carrier family 35 member A1 (SLC35A1 KD; Pool 4). These recombinant Fc-F241A glycovariants were purified by size exclusion chromatography (SEC) and suspended in phosphate buffered saline (PBS). N-glycan profiling was performed by high-performance liquid chromatography (HPLC) followed by mass spectrometry (MS) using known standards to define the structure of the biantennary N-glycan attached to Asn297.

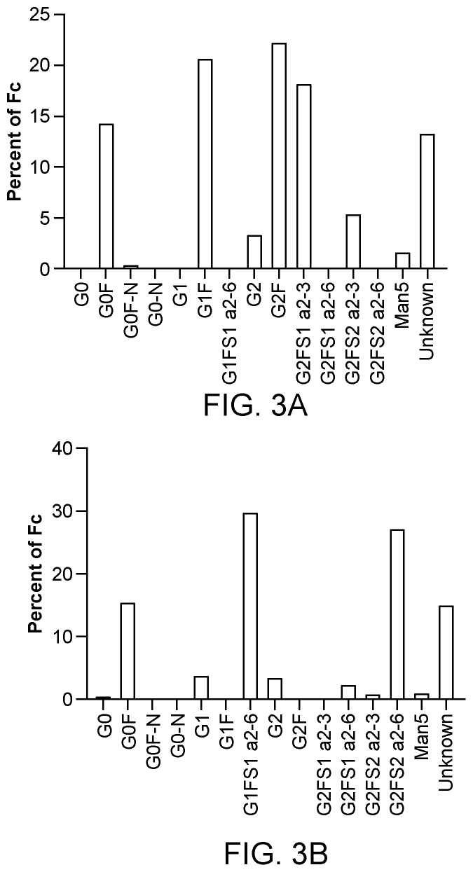

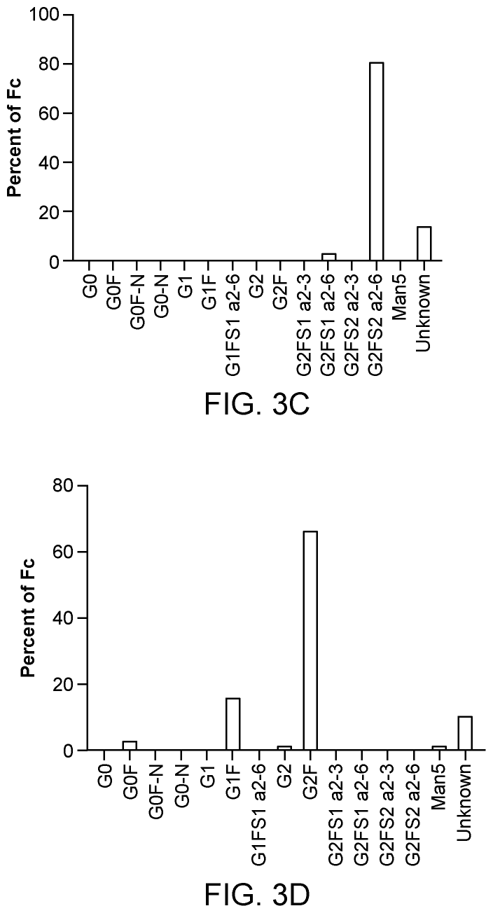

Data were represented as the percent of the Fc molecules that contain the defined glycan structure from the 1 L CHO cultures. On the x-axis, glycan structure is defined as G0 (terminating with GlcNAc), G0F (terminating with GlcNAc with fucose), G1 (terminating with one galactose), G1F (terminating with one galactose and a fucose), G2 (terminating with two galactose), G2F (terminating with two galactose and a fucose), G2FSA (terminating with one sialic acid and a fucose), and G2FSA2 (terminating with two sialic acids and a fucose). The sialic acid is added via the α(2,3) linkage in Pool 1, and the α(2,6) linkage in Pools 2 and 3. When the recombinant human IgG1 Fc domain bearing F241A was expressed alone in CHO cells (Pool 1), 18% of the Fc polypeptides in the pool were mono-α(2,3) sialylated, 5% di-α(2,3) sialylated, and 47% with terminal galactose (FIG. 3A). When co-expressed with ST6GAL1 (Pool 2), the Fc polypeptides were 30% mono-α(2,6) sialylated, 27% di-α(2,6) sialylated, and 7% with terminal galactose (FIG. 3B). When co-expressed with ST6GAL1 and B4GALT1 (Pool 3), the Fc polypeptides were 3% mono-α(2,6) sialylated, 81% di-α(2,6) sialylated, and 0% with terminal galactose (FIG. 3C). When co-expressed with siRNA targeting SLC35A1 (Pool 4), the Fc domain was entirely devoid of sialic acid, with 84% of the Fc N-glycans terminating with galactose (FIG. 3D).

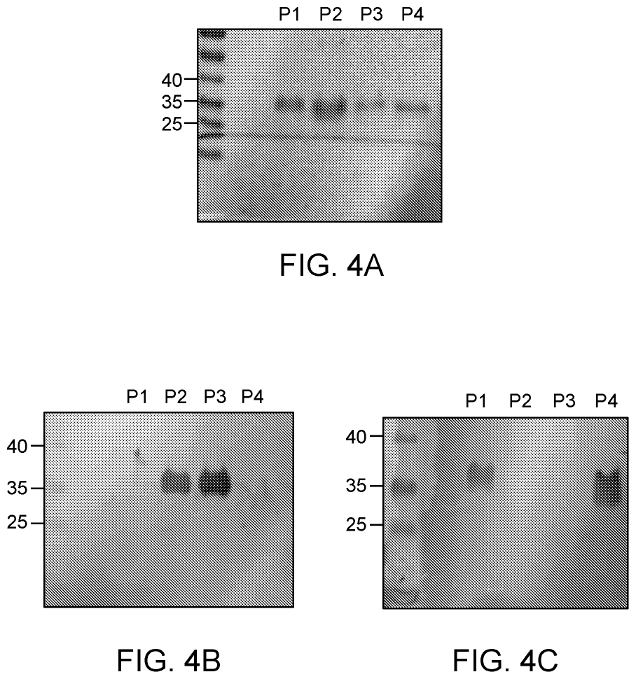

To independently verify the glycan structure associated with the above-described Pools 1-4 generated from the 1 L stable CHO cultures, recombinant Fc-F241A glycovariants were purified by size exclusion chromatography and suspended in PBS. 5 g of each of Fc-F241A glycovariant pools P1, P2, P3, and P4 was resolved by SDS-PAGE under reducing conditions and Coomassie stained (FIG. 4A), transferred to nitrocellulose membrane and incubated with biotinylated sambucus nigra (SNA) lectin to detect α(2,6) sialic acid (FIG. 4B), or transferred to nitrocellulose membrane and incubated with biotinylated MAL I lectin to detect terminal galactose (FIG. 4C). Lectins were detected using ALP-conjugated goat anti-biotin antibody. These findings are consistent with the HPLC/MS analysis, confirming that pools 2 and 3 uniquely contain 2,6 sialic acid, whereas pools 1 and 4 do not, but rather present with more terminal galactose.

### Example 3: Fc Sialylation Improves Exposure to Modified Fc Polypeptides in Mice

To determine the pharmacokinetics of sialylated F241A Fc mutant polypeptides in vivo, 7-8 week-old female JAX-014565 mice (mouse FcRN homozygous knock-out, human FcRN hemizygous, Tg32 strain) were separated into 3 groups of 6 mice each. Mice received a single intravenous (IV) bolus administration via tail vein of 20 mg/kg recombinant human IgG1 Fc domain-bearing F241A (stable CHO 10 mL cultures) from Pool 2 (66% α(2,6) sialylated), Pool 3 (93% α(2,6) sialylated), and Pool 4 (0% sialylated). Blood was collected at each of 7 time points (3 mice per time point) at pre-dose, 0.5 hour, 1, 3, 7, 21, and 35 days. Serum was prepared following standard procedures and transferred to Eppendorf tubes for storage. The concentration of F241A Fc polypeptides in each serum sample was determined by ELISA. Briefly, 96-well plates were coated overnight with 1 g anti-human Fc-specific capture antibody at 4° C. Plates were washed 3 times with blocking buffer, followed by incubation with blocking buffer (80 L per well) for 2 hours at room temperature with shaking (500 rpm). Plates were again washed, and standards were diluted (starting at 300 ng/mL with serial 1:3 dilutions for 7 additional concentrations) and applied to the plate to generate the calibration curve. Samples were diluted in dilution buffer as appropriate (minimum of 20× dilution) and applied to the same plate, then incubated for 2 hours at room temperature with shaking (500 rpm). The plate was washed and incubated for one hour at room temperature (500 rpm shaking) with the detection antibody diluted 1:50,000. Washed plates were then developed by adding 25 L/well of TMB substrate and incubated for 5-10 minutes. Quenching was achieved by adding 25 L/well 4N sulfuric acid, and absorbance at 450 nm was read using a plate reader. Graphical analysis was performed using Prism software and area under the curve (AUC) was determined.

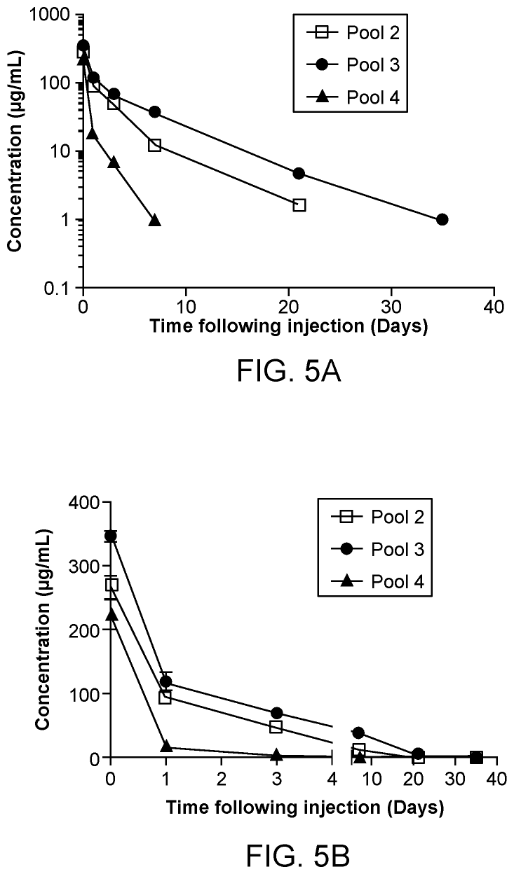

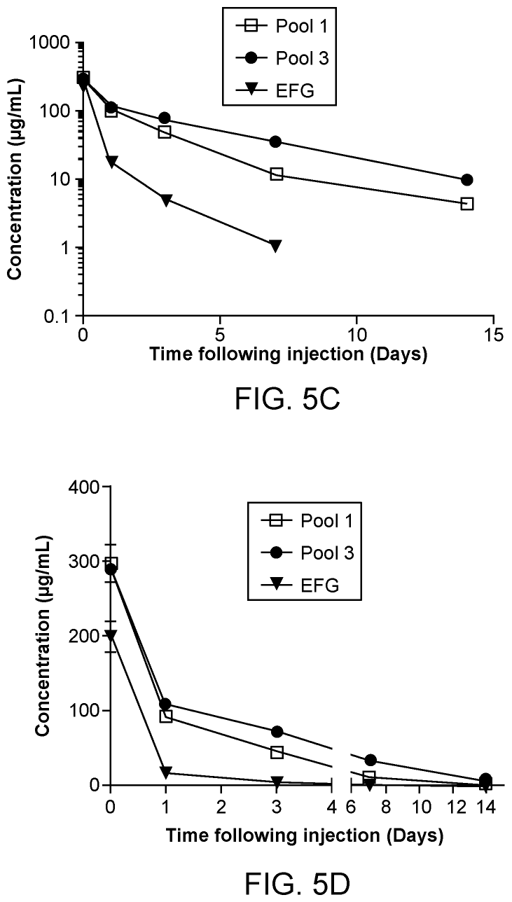

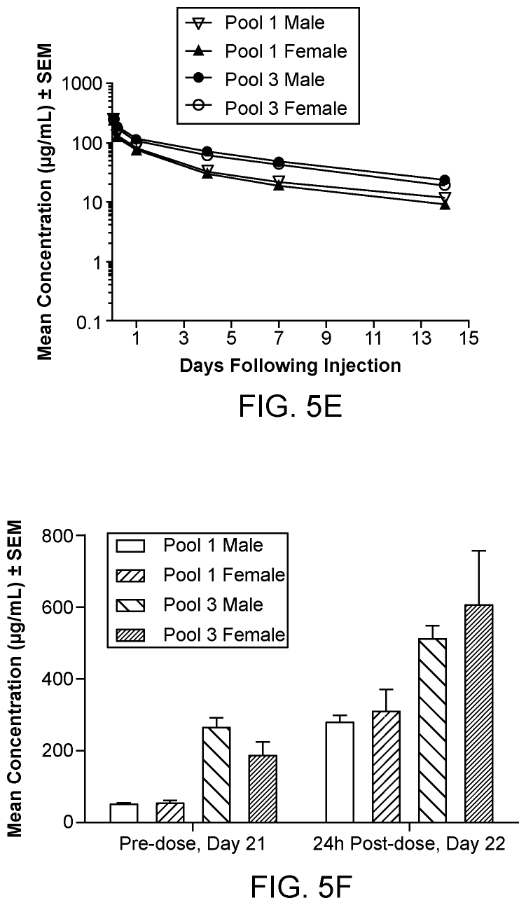

Serum concentration (g/mL) was depicted over time (days) for each of Fc-F241A Pool 2 (open square), Pool 3 (closed circle), and Pool 4 (closed triangle) in FIGS. 5A-5B. Data was represented with concentration on a log scale (FIG. 5A) and linear scale (FIG. 5B). These pools were compared directly as a means of evaluating the impact of the extent of α(2,6) sialylation on pharmacokinetics in human FcRN mice. The AUC for each of these Fc-F241A glycovariants is summarized in Table 1, below, revealing that the extent of sialylation is directly proportional to exposure in these mice. The non-sialylated Fc Pool 4 had the lowest AUC, followed by the 66% α(2,6) sialylated Pool 2. The greatest exposure was achieved with the 93% α(2,6) sialylated Fc Pool 3.

TABLE 1
Pharmacokinetics of α(2,6) sialylated
F241A Fc mutant polypeptides

| | | AUC |
|---|---|---|
| Fc Pool | Glycovariant | (day*mg/mL) |
| Pool 4 (F241A + SLC35A1 KD) | 0% sialylated | 118 |
| Pool 2 (F241A + ST6GAL1) | 66% α(2,6) sialylated | 483 |
| Pool 3 (F241A + ST6GAL1 + | 93% α(2,6) sialylated | 856 |
| B4GALT1) | | |

To compare the differences in pharmacokinetics between α(2,3) and α(2,6) sialylated F241A Fc mutants, 7-8-week-old female JAX-014565 mice were separated into three groups of 6 mice each. Mice received a single IV bolus administration via tail vein of recombinant human IgG1 Fc domain-bearing F241A (20 mg/kg) from stable 10 mL CHO cultures of Pool 1 (35% α(2,3) sialylated) or Pool 3 (93% α(2,6) sialylated) material, and the human IgG1 Fc domain (efgartigimod (EFG)) bearing the Abdeg mutations (M252Y/S254T/T256E/H433K/N434F) to enhance FcRn binding (0% sialylated), which was expressed transiently by CHO cells. Blood was collected at each of 6 time points (3 mice per time point) at pre-dose, 0.5 hour, 1, 3, 7, and 14 days. Serum was prepared following standard procedures and transferred to tubes for storage. The concentration of F241A Fc in each serum sample was determined by ELISA, as described above.

Concentration-time profiles are shown for each of Fc-F241A Pool 1, Pool 3, and EFG Fc (FIGS. 5C-5D). Data is graphed with concentration on a log scale (FIG. 5C) and linear scale (FIG. 5D). The observed AUC difference between the α(2,3) and α(2,6) sialylated Fc-F241A are likely due to differences in extent of sialylation, rather than a α(2,3) versus α(2,6) sialylation difference, given a similar affect observed when titrating the level of α(2,6) sialylation. The 93% α(2,6) sialylated Fc-F241A exhibited the greatest exposure in these mice. The EFG Fc domain had the lowest AUC, similar to non-sialylated F241A (Pool 4) as shown elsewhere in FIGS. 5A-5B. The AUC values for each Fc pool are provided in Table 2, below.

TABLE 2
Pharmacokinetics of α(2,3) and α(2,6)
sialylated F241A Fc mutant polypeptides

| | | AUC |
|---|---|---|
| Fc Variant | Glycovariant | (day*mg/mL) |
| Pool 1 (F241A Only) | 35% α(2,3) sialylated | 460 |
| Pool 3 (F241A + ST6GAL1 + | 93% α(2,6) sialylated | 707 |
| B4GALT1) | | |
| EFG | 0% sialylated | 104 |

Similar experiments were then performed in male and female CD1 mice as follows, using the stable CHO 1 L culture Fc-F241A material. 5-7 week-old male and female CD1 mice were separated into two groups (6 males and 6 females each) and dosed via single IV bolus injection with 20 mg/kg of Pool 1 Fc-F241A (23% α(2,3) sialylated) or Pool 3 Fc-F241A (84% α(2,6) sialylated). Blood was collected by retro-orbital bleed at each of 6 time points (3 mice per time point) at pre-dose, 1 hour, 4 hour, and 1, 3, 7, and 14 days post-dose. Serum was prepared following standard procedures and transferred to tubes for storage. The concentration of F241A in each serum sample was determined by ELISA, as described above.

As shown in the concentration-time profile (FIG. 5E) and Table 3, below, there was no appreciable difference in exposure between male and female mice receiving either α(2,3) sialylated (open and closed triangles) or α(2,6) sialylated (open and closed circles) Fc-F241A. As observed previously, however, the α(2,6) sialylated material had greater exposure relative to α(2,3) sialylated, although this difference is likely attributed to the extent of sialyation as opposed to the α(2,3) versus α(2,6) linkage.

TABLE 3
Pharmacokinetics of α(2,3) and α(2,6)
sialylated F241A Fc mutant polypeptides

| | | AUC |
|---|---|---|
| Human Fc | Sialylation | (day*mg/mL) |
| Pool 1 Male | 23% α(2,3) sialylated | 475 |
| Pool 1 Female | 23% α(2,3) sialylated | 436 |
| Pool 3 Male | 84% α(2,6) sialylated | 814 |
| Pool 3 Female | 84% α(2,6) sialylated | 774 |

Finally, effects of repeat dosing of α(2,6) sialylated Fc-F241A on exposure to the Fc-241A was tested in CD1 mice. 5-7 week-old male and female CD1 mice were separated into three groups (6 male and 6 female each) and dosed (4-weekly IV bolus injections) with 100 mg/kg of Pool 1 Fc-F241A (23% α(2,3) sialylated) or 100 mg/kg Pool 3 Fc-F241A (84% α(2,6) sialylated). Blood was collected immediately prior to the 4^{th} dose on day 21, and 24 hours following the 4^{th} dose on day 22. Serum was prepared following standard procedures and transferred to tubes for storage. The concentration of F241A in each serum sample was determined by ELISA, as described above.

Greater exposure at both Cₘᵢₙ and 24 hours following the 4^{th} dose was achieved in male and female CD1 mice following dosing with the α(2,6) sialylated Fc-F241A relative to the α(2,3) sialylated Fc-F241A (FIG. 5F). Notably, 100 mg/kg of the α(2,6) sialylated material achieved minimum exposures greater than 200 μg/mL which is double of the concentration required for engagement of the DC-SIGN mechanism in cell-based assays.

### Example 4: α(2,6) Sialylation of F241A Fc Improves Potency of Anti-Inflammatory Activity in a Murine Model of Arthritis

To determine the therapeutic efficacy of sialylated F241A Fc mutant polypeptides in inflammatory disease, a murine model of arthritis was employed. KRN transgenic heterozygous mice on the C57BL/6 background were bred with NOD/SHiLtJ to generate K/BxN mice. Serum collection from animals with inflamed joints was pooled from mice that spontaneously develop inflammation (6-9 weeks of age). The pooled serum was frozen in aliquots and used for all experiments presented here. Joint inflammation was generated in C57BL/6 mice by intravenous injection of 4-200 L pooled K/BxN serum. Symptoms of arthritis were scored daily as 0 (unaffected), 1 (swelling of one joint), 2 (swelling of more than on joint), and 3 (severe swelling of the entire paw). Each paw was scored, and the sum of all four paws was used to generate the clinical score. Scores generated from the two independent technicians were averaged to generate the final clinical score. Fc-F241A produced by transient transfection in CHO cells was used in these experiments. For treatment conditions, mice received a single administration of 1 g/kg IVIG or 50 mg/kg of (41%) α(2,3) sialylated F241A (transiently expressed in CHO cells) via tail vein bolus injection 1 hour prior to administration of the KBxN serum.

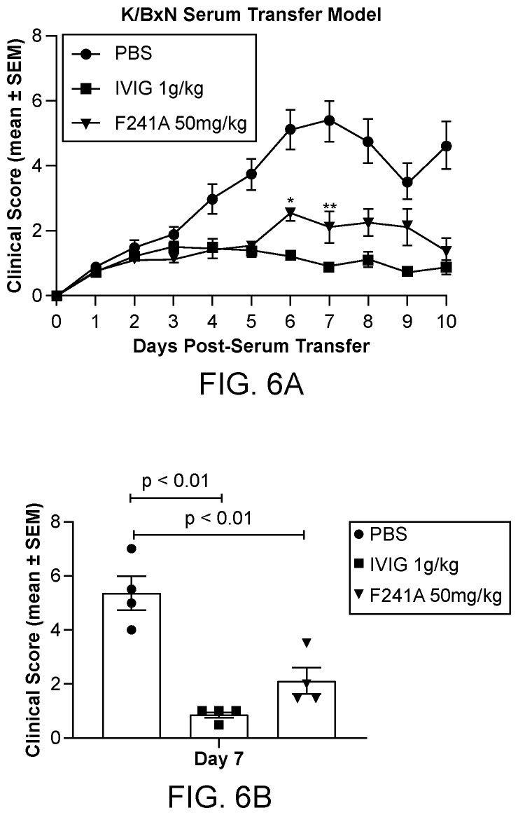

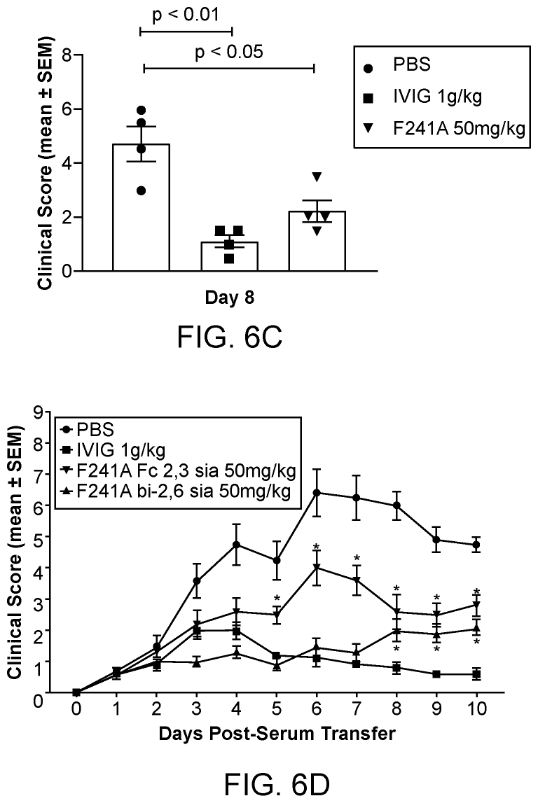

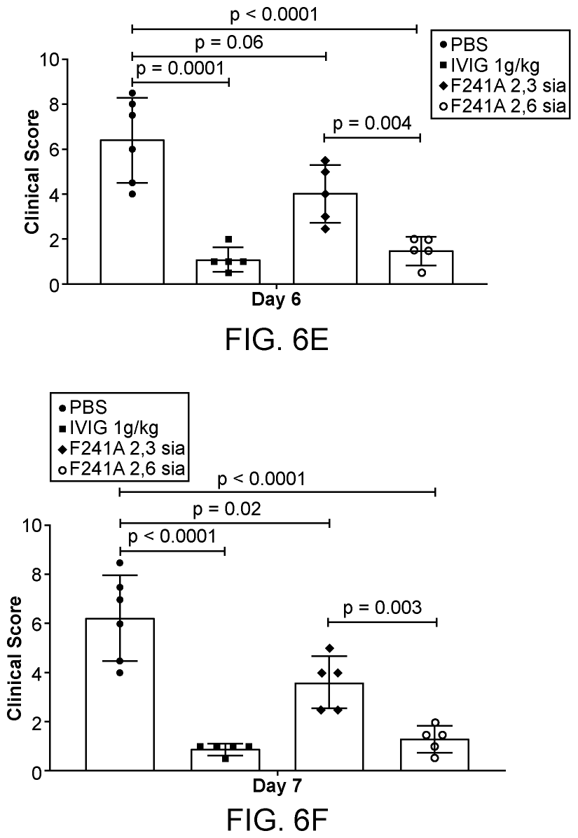

Mice (4 per group) were randomized for treatment with a single administration of 1 g/kg IVIG or 50 mg/kg F241A. Clinical scores for IVIG- and F241A Fc-treated groups were not significantly different from each other except on days 6 and 7, as determined by unpaired t-test (FIG. 6A). Peak inflammation was achieved by day 7 in the PBS-treated mice. Clinical scores on study days 7 and 8 were, therefore, shown separately in FIGS. 6B-6C as box plots, represented as mean and standard error of the mean. Both 1 g/kg IVIG and 50 mg/kg F241A significantly suppressed inflammation relative to PBS. These data suggest that F241A is approximately 20-fold more potent than IVIG in the K/BxN serum transfer model of arthritis.

Similar experiments using the Fc-F241A stable CHO 1 L culture material were then repeated using 50 mg/kg (84%) α(2,6) sialylated F241A Fc. The two different F241A glycoforms evaluated here differed in two ways. First, one recombinant human IgG1 Fc domain contained α(2,3)-linked SA (Pool 1), whereas the other contained α(2,6)-linked SA (Pool 3). Second, the percent of Fc molecules that contain SA, as determined by HPLC, approximately 23% of the α(2,3)-linked material contained SA, whereas approximately 84% of the α(2,6)-linked material contained SA. Mice (5-6 per group) were randomized for treatment with a single administration of 1 g/kg IVIG, 50 mg/kg of α(2,3) sialylated F241A Fe, or 50 mg/kg of α(2,6) sialylated F241A Fc. IVIG demonstrated significantly greater anti-inflammatory activity than F241A α(2,3) sialylated Fc from day 5 and beyond, as determined by unpaired t-test (p<0.01; FIG. 6D). By comparison, IVIG was only significantly different than F241A α(2,6) sialylated Fc from days 8 and beyond (unpaired t-test; p<0.05). At the peak of inflammation for PBS-treated animals, days 6 and 7, the α(2,6) sialylated F241A Fc resulted in significantly less inflammation relative to the α(2,3) sialylated F241A Fc (FIGS. 6E-6F).

### Example 5: Sialylated F241A Fc Polypeptides are More Potent than IVIG in a Murine Model of Idiopathic Thrombocytopenic Purpura

To determine the therapeutic efficacy of sialylated F241A Fc mutant polypeptides in another inflammatory disease, a murine model of idiopathic thrombocytopenic purpura (ITP) was employed. The mouse 6A6-IgG2a anti-mouse platelet antibody was produced in 293T cells by transient transfection in serum-free media, then purified with protein G beads. Female C56BL/6 mice 6-8 weeks of age (n=5 per group) received a single tail vein injection of 4 g 6A6 antibody at time zero. Mice were randomized to receive PBS, 1 g/kg IVIG, or 100 mg/kg F241A Fc bearing α(2,3) sialylation (F241A α(2,3) sia), transiently expressed in CHO cells, 41% sialylated) via tail vein injection 2 hours prior to 6A6 administration. Platelet counts were monitored prior to 6A6 administration on study day 0, and again at 24, 48, and 72 hours post 6A6 administration. A box and whisker plot representing the first and third quartiles, median, and minimum and maximum values was generated.

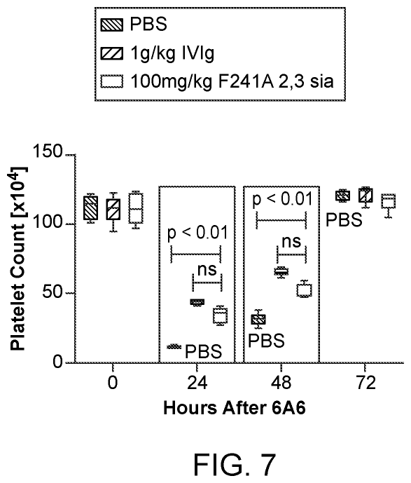

In this model of ITP, baseline blood platelet counts were similar across all three treatment groups on day 0 (FIG. 7). Following administration of the 6A6 anti-platelet antibody, platelets were >90% depleted by 24 hours in the PBS control mice, returning to ~40% of pre-6A6 administration levels by 48 hours, and returning to normal by 72 hours. Treatment with 1 g/kg IVIG or 100 mg/kg α(2,3) sialylated F241A Fc resulted in statistically significant protection from platelet loss (p<0.01) at 24 and 48 hours post 6A6 administration, as determined by unpaired t-test. These findings demonstrate that α(2,3) sialylated F241A Fc is approximately 10-fold more potent than IVIG in this model of ITP.

### Example 6: Terminal Sialylation of F241A Fc Enhances Half-Life and Bioavailability in Vivo

Full-length IgGs are known to have an extended serum half-life of up to 21 days due to interactions with FcRn, which recycles IgG into the circulation after cellular uptake. In contrast, Fc fragments have are known to have a shorter serum half-life. Therefore, to test whether sialylation of F241A Fe impacted its serum half-life, a single intravenous 20 mg/kg dose of each of the different F241A Fc glycoforms or wild-type (WT) Fc was administered to humanized FcRn Tg32 (hFcRn) mice, and the amount of human IgG1 Fc in circulation was quantified over time. Mice were randomly assigned to receive a single intravenous injection of 20 mg/kg of one glycoform of F241A Fc, with 6 mice per group. Blood was collected for serum isolation by retro-orbital bleeding at 0 min, 30 min, day 1, day 5, day 7, day 14, day 21, and day 35 post-administration. Mice were euthanized and blood collected via cardiac puncture. Depending on the exposure achieved, later time points were not plotted if values were below the limit of quantitation (1 μg/mL). Not all mice were bled at every time point, but each mouse contributed 3-4 times over the course of the study. Serum samples were stored at −80° C. prior quantitation of concentration of F241A Fc in the mouse serum using ELISA.

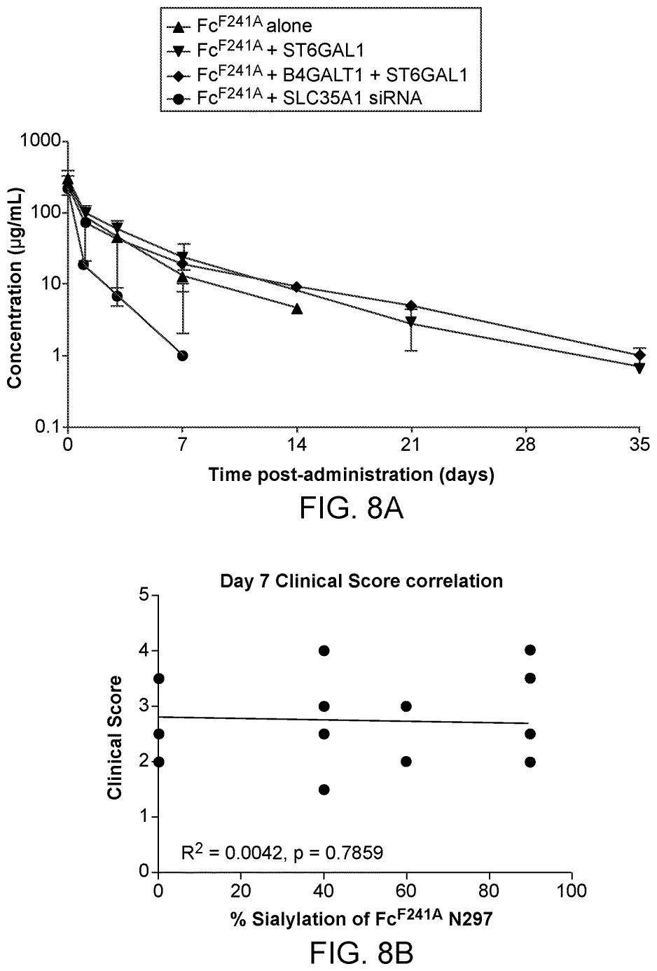

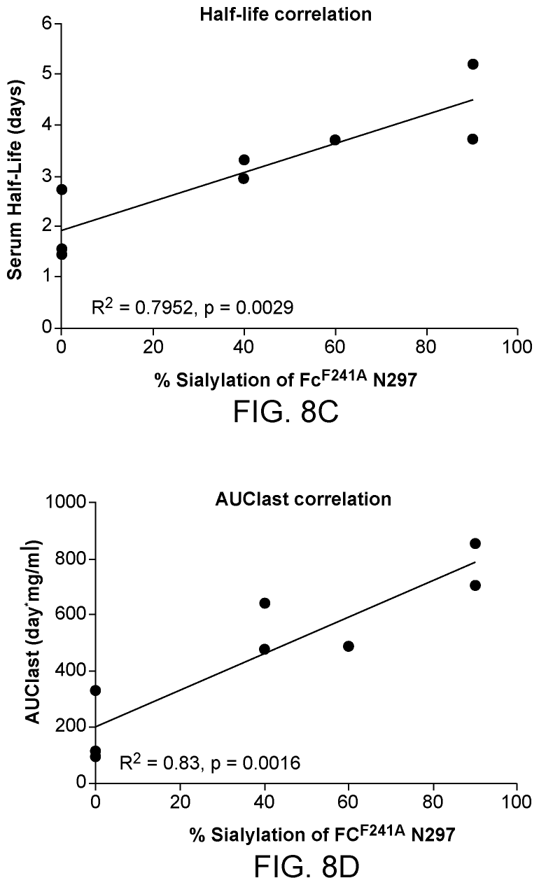

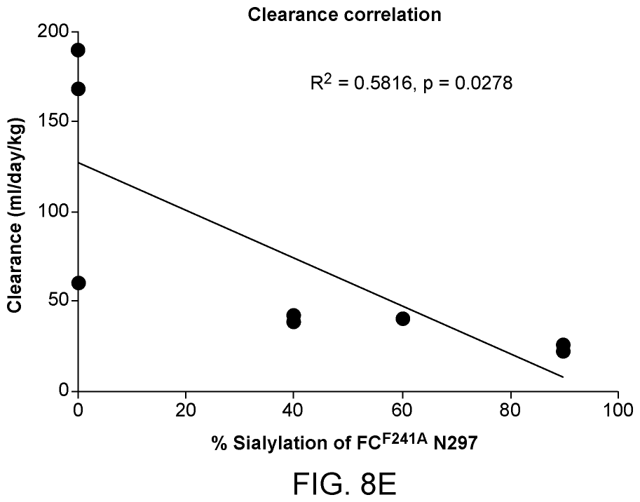

Following administration, both asialylated WT Fc and F241A Fc produced in cells expressing short interfering RNA (siRNA) against the Golgi apparatus sialic acid transporter, SLC35A1 (F241A/siSLC Fc) were quickly cleared from circulation and undetectable after 14 and 10 days, corresponding to a half-life of 2.71 and 1.43 days, respectively (FIG. 8A and Table 4). These preparations had an area under the plasma concentration-time curve to the last measurable plasma concentration (AUClast) of 322 and 118 day*mg/mL, respectively, and clearance rates of 61.3 and 168.3 mL/day/kg (FIG. 8A and Table 4). F241A Fc had a half-life of 2.96 days, AUClast of 297 day*mg/mL, and clearance of 42.1 mL/day/kg (FIG. 8A and Table 4). F241A Fc produced in cells expressing the α-2,6-sialyltransferase ST6GAL1 (F241A/ST6 Fc) had a 3.72 day serum half-life, a 483 day*mg/mL AUClast, and a clearance of 40.8 mL/day/kg. F241A Fc produced in cells expressing the β1,4-galactosyltransferase, B4GALT1, alongside ST6GAL1 (F241A/B4ST6 Fc) exhibited the longest residency in the serum with a half-life of 5.2 days, and only fell below a concentration of 1 μg/mL past day 35 post-administration, with a AUClast of 782 day*mg/mL and clearance rate of 24.8 mL/day/kg (FIG. 8A and Table 4).

TABLE 4
Half-life and bioavailability of F241A glycoforms Fc in vivo

| | | Half-life | AUClast | Clearance |
|---|---|---|---|---|
| Transfection | Sialylation | (days) | (day*mg/mL | (mL/day/kg) |
| WT hIgG1 Fc | 0% | 2.71 | 322 | 61.3 |
| F241A Fc alone | 40% α-2,3 | 2.96 | 297 | 42.1 |
| F241A Fc + | 60% α-2,6 | 3.72 | 483 | 40.8 |
| ST6GAL1 | | | | |
| F241A Fc + | 90% α-2,6 | 4.47 | 782 | 24.8 |
| B4GALT1 + | | | | |
| ST6GAL1 | | | | |
| F241A Fc + | 0% | 1.43 | 118 | 168.3 |
| SLC35A1 | | | | |
| siRNA | | | | |

Although percent sialylation of the N297 glycan on F241A Fe exhibited no correlation with anti-inflammatory activity (R²=0.0042; FIG. 8B), there was a significant positive correlation with half-life (R²=0.7952; FIG. 8C), AUClast (R²=0.83; FIG. 8D), and significant negative correlation with clearance rate (R²=0.5816; FIG. 8E). These results were surprising as IgG half-life is thought to be primarily regulated by FcRn. Furthermore, the Fc glycan is thought to be buried in a pocket at the top of the CH2 domain (FIG. 9B), and largely inaccessible. Based on these results, the F241A/B4ST6 Fc preparation with ~90% α-2,6-sialylation on the N297 glycan was selected for use in further analyses and experiments.

### Example 7: Preferential Receptor Engagement Distinguishes F241A Fc and Fc^{Abdeg}

Fc^{Abdeg} (Efgartigimod) is a recently FDA-approved therapeutic that accelerates the depletion of circulating total IgG by saturating FcRn and is currently approved for the treatment myasthenia gravis. Fc^{Abdeg} contains “Abdeg” mutations (M252Y, S254T, T256E, H433K, and N434F) in the region of the Fc that binds to FcRn, thereby allowing it to outcompete native IgG (FIG. 9A and FIG. 9B). The following experiments were conducted to confirm if Fc^{Abdeg} and F241A/B4ST6 Fc functioned through similar receptors and cellular pathways.

At the outset, the interactions between WT Fc, F241A/B4ST6 Fc, and Fc^{Abd}eg were tested with mouse and human FcRn using surface plasmon resonance (SPR). WT Fc and F241A/B4ST6 Fc bound similarly to mouse (FIG. 9C) and human (FIG. 9D) FcRn. In contrast, the dissociation constant (KD) of Fc^{Abdeg} was 267-fold and 39-fold lower than F241A/B4ST6 Fc to mouse FcRn (FIG. 9C) and human FcRn (FIG. 9D), respectively. Subsequently, the effects of both mutant Fcs on circulating mouse IgGs through FcRn were examined. C57BL/6 and hFcRn (Tg32) mice were given a single intravenous injection of F241A/B4ST6 Fc or Fc^{Abd}eg and, following dosing, serum mIgG was determined via ELISA. In wild-type C57BL/6 mice with murine FcRn, F241A/B4ST6 Fc treatment did not reduce serum IgG titers (FIG. 9E). In contrast, Fc^{Abdeg} induced a significant immediate and lasting reduction in IgG titers (FIG. 9E). F241A/B4ST6 Fc did not impact IgG serum titers in hFcRn (Tg32) mice (FIG. 9F). However, Fc^{Abdeg} induced a transient drop in IgG titers on day 1. These results are consistent with the high affinity of Fc^{Abdeg} for mFcRn, and relatively lower affinity for hFcRn, requiring multiple doses in humans to trigger long-term reductions in serum IgG.

Both IVIG and sialylated IgG Fc require the murine C-type lectin Specific ICAM3-Grabbing Non-Integrin Related-1 (SIGN-R1) to mediate anti-inflammatory activity in vivo. This requirement can be circumvented by introduction of SIGN-R1 human orthologue, Dendritic Cell-Specific ICAM-3-Grabbing Non-Integrin (DC-SIGN). Therefore, the ability of both F241A/B4ST6 Fc and Fc^{Abdeg} to bind DC-SIGN was examined via a cell-binding assay. Immortalized bone marrow-derived macrophages (BMDMs) were generated from SIGN-R1^{-/-} and hDC-SIGN⁺/SIGN-R1^{-/-} mice, and the cells were incubated with PBS, F241A/B4ST6 Fc, or Fc^{Abdeg} Staining for hCD209 using an Alexa Fluor 647 anti-hCD209, clone 9E9A8 antibody confirmed the presence of the receptor by flow cytometry. Cells were cultured overnight in high glucose DMEM medium supplemented with 1× antibiotic-antimycotic and 1×FBS at 37° C. and 5% CO₂. The next day, supernatants were discarded and cells were detached with enzyme-free dissociation buffer. Suspended cells were collected and centrifuged for 10 minutes at 400 rpm. The pellet was resuspended and washed in binding buffer (1×TBS, 1 nM CaCl₂, 2.5% FBS and 0.05% sodium azide). Fc receptors were blocked by adding anti-mouse CD16/32 antibody clone 93. After blocking, cells were washed in binding buffer, after which 100 μg/mL of Fc^{Abdeg} and F241A/B4ST6 Fc were added to the cells, which were then incubated for 1 hour on ice. Afterwards, cells were washed again with binding buffer. Because Fc^{Abdeg} and F241A/B4ST6 Fc are both IgG Fc-based constructs, the cells were then stained with either an APC- or PE-conjugated antibody against hIgGFc, clone M1310G05. Staining was performed for 30 minutes on ice in the dark. After staining, cells were washed with binding buffer and fixed in 2% formaldehyde before analysis. BMDMs were analyzed by flow cytometry to detect surface-bound IgG. No bound IgG were detected on the SIGN-R1^{-/-} BMDMs incubated with F241A/B4ST6 Fc or Fc^{Abdeg} (FIG. 9G). However, IgG was detected on hDC-SIGN⁺/SIGN-R1^{-/-} BMDMs treated with F241A/B4ST6 Fc, but not with Fc^{Abdeg} (FIG. 9G).

For in vivo assessment of mechanistic differences in the activity of F241A/B4ST6 Fc and Fc^{Abdeg} C57BL/6 and SIGN-R1^{-/-} mice were administered PBS, high-dose IVIG, F241A/B4ST6 Fc, or Fc^{Abdeg} followed by K/BxN sera, and tracked paw swelling over 10 days (FIG. 9H-9J). A clinical score of 0-3 was given to each paw, with a score of 0 representing no inflammation in the paw and a score of 3 representing severe inflammation of all joints in the paw, and the average score for all fours paw on each mouse in treatment groups is shown. In WT mice, marked paw swelling was observed only in the PBS-treated mice (FIG. 9H and FIG. 9J). In SIGN-R1^{-/-} mice, swelling was observed in PBS, IVIG, and F241A/B4ST6 Fc-treated mice (FIG. 9I and FIG. 9J). However, SIGN-R1^{-/-} mice administered Fc^{Abdeg} had significantly less inflammation, as measured by clinical score, including at the peak of disease (day 7; FIG. 9J). Taken together, these results demonstrate that F241A/B4ST6 Fc and Fc^{Abdeg} suppress autoantibody-induced inflammation through distinct molecular pathways.

### Example 8: Combinatorial Anti-Inflammatory Activity of F241A/B4ST6 Fc and Fc^{Abdeg}

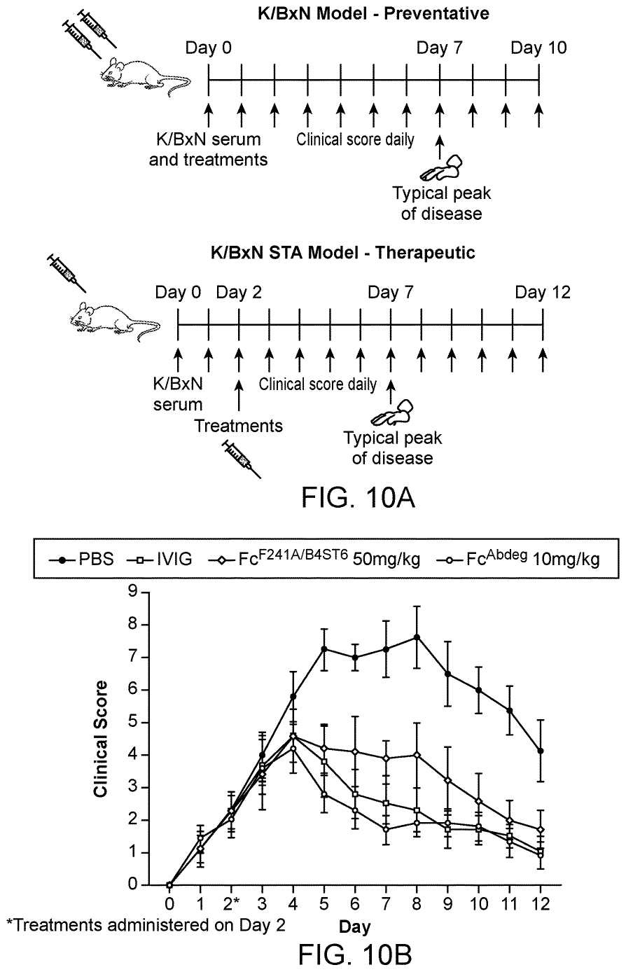

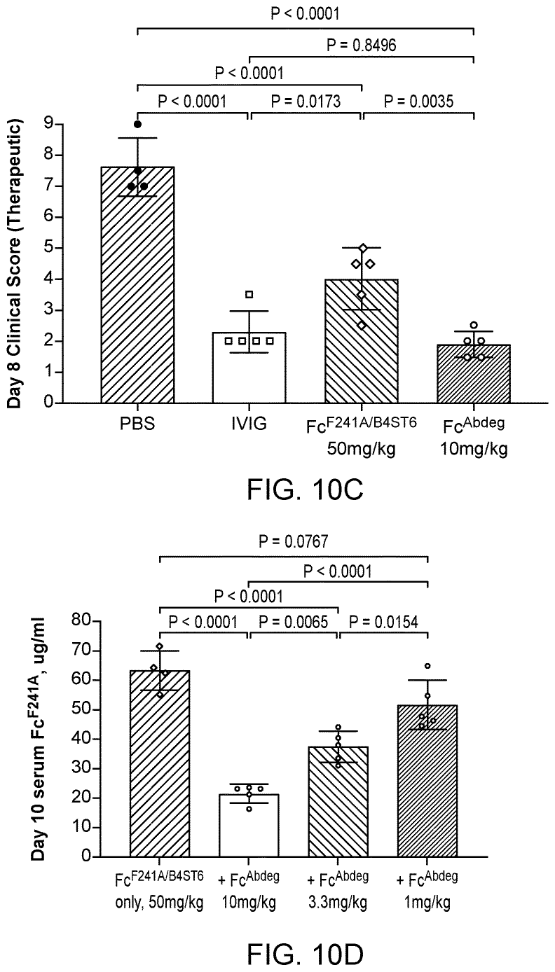

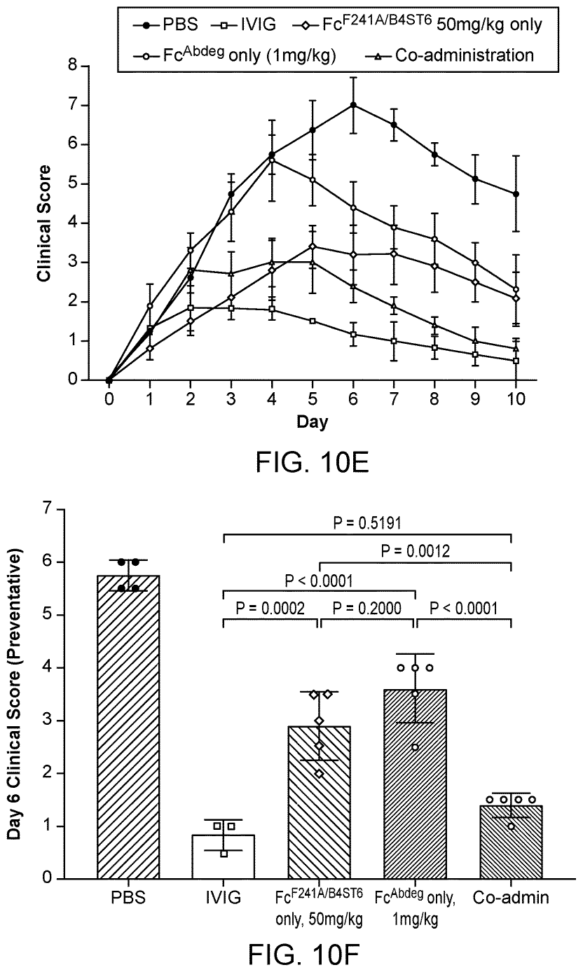

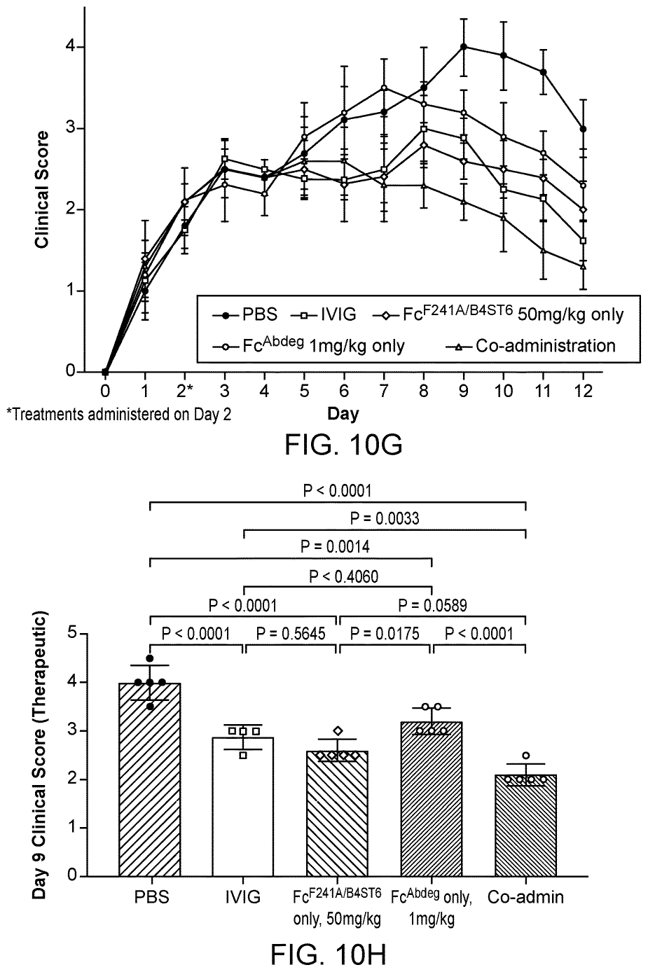

To examine whether F241A/B4ST6 Fc and Fc^{Abdeg} could effectively attenuate autoantibody-induced inflammation when co-administered in vivo, the effects of F241A/B4ST6 Fc and Fc^{Abdeg} was assessed in the K/BxN model in both a preventative (FIG. 10A, top panel) and therapeutic (FIG. 10A, top panel) manner. Both mutant Fcs significantly protected against inflammation in a manner comparable to high-dose IVIG independent of timing of administration (FIG. 9J, FIG. 10B, and FIG. 10C).

Because Fc^{Abdeg} suppresses IgG-mediated inflammation by saturating FcRn, thereby reducing total IgG titers and its markedly enhanced affinity for mFcRn, Fc^{Abdeg} was tested for its ability to lead to more rapid clearance of F241A/B4ST6 Fc from circulation. To determine a dose combination at which the clearance effects of Fc^{Abdeg} would not interfere with the activity or half-life of F241A/B4ST6 Fc, C57BL/6 mice were administered with 50 mg/kg of F241A/B4ST6 Fc in combination with decreasing doses of Fc^{Abdeg} and measured human Fc titers at day 10 post-administration (FIG. 10D). Fc^{Abdeg} caused a decrease in circulating F241A/B4ST6 Fc in a dose-dependent manner, as measured by ELISA. However, Fc^{Abdeg} at I mg/kg, i.e., one-tenth of the clinical dose, induced the least clearance of circulating F241A/B4ST6 Fc.

Because Fc^{Abdeg} displays at least a 10-fold enhanced affinity for mouse FcRn compared to human FcRn, it was reasoned that 10-fold lower dosing would be more representative of the human system and would not deplete F241A/B4ST6 Fc titers. Thus, C57BL/6 mice were administered PBS, high-dose IVIG, 50 mg/kg F241A/B4ST6 Fc, 1 mg/kg of Fc^{Abdeg} or 50 mg/kg F241A/B4ST6 Fc and 1 mg/kg of Fc^{Abdeg} together, followed by K/BxN sera. In the preventative model, IVIG, 50 mg/kg F241A/B4ST6 Fc, and 1 mg/kg of Fc^{Abdeg} all effectively reduced inflammation as compared to PBS-treated mice (FIG. 10E and FIG. 10F). Surprisingly, mice who received the co-administered F241A/B4ST6 Fc and Fc^{Abdeg} exhibited a greater reduction in inflammation than either Fc alone.

The combined effects of the aforementioned Fc mutants as anti-inflammatory biologicals were further tested in the therapeutic K/BxN model. As above, mice were administered arthritogenic K/BxN sera on day 0, and PBS, high dose IVIG, 50 mg/kg F241A/B4ST6 Fc, 1 mg/kg of Fc^{Abdeg} or 50 mg/kg F241A/B4ST6 Fc and 1 mg/kg of Fc^{Abdeg} on day 2, and foot swelling was monitored over the next several days (FIG. 10G and FIG. 10H). The co-administration reduced inflammation by day 6, 4 days after treatment. Unexpectedly, this was significantly different from treatment with either Fc alone. These data indicate that combined administration of F241A/B4ST6 Fc and Fc^{Abdeg} results in potentiation of the anti-inflammatory response in vivo, as compared to the effects of either Fc alone.

### Example 9: Efficacy of Fc^{F241A} in an Antibody Transfer Model of Pemphigoid Disease

Autoantibody-mediated pemphigoid disease was modeled in mice by repeat administration of rabbit anti-mouse type VII collagen polyclonal antibody (pathogenic autoantibody found in epidermal bullosa acquisita). Treatment was administered on study day 0 (3 hours prior to first administration of pathogenic autoantibody) and again on day 3.

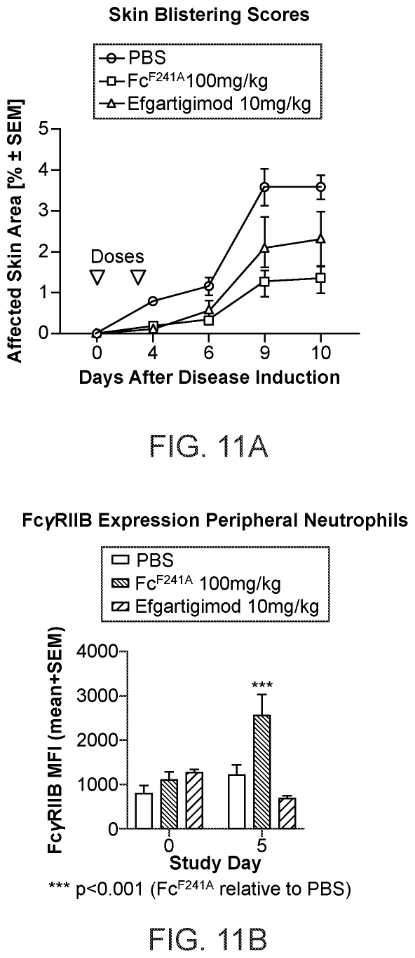

Using this model of pemphigoid disease, we evaluated the potential for 100 mg/kg Fc^{F241A} relative to the FcRN inhibitor efgartigimod (10 mg/kg) to prevent autoantibody-mediated blister formation. Protection against autoantibody-mediated skin blistering over time is presented in FIG. 11A. As shown, when treated with PBS control (open circles), the percentage of total skin area affected by blisters rises from ~1% on day 4 to 3-4% on days 9-10. When treated with Fc^{F241A}, maximum blister formation of 1-2% is observed on days 9-10, representing a marked decrease in blister formation relative to PBS control. Efgartigimod resulted in a more modest degree of disease control, with maximum blister formation of 2-3% observed on days 9-10.

In this model, immune complexes are forming with the collagen type VII antigen in the basal membrane zone resulting in crosslinking of FcγRIV receptor on the surface of infiltrating neutrophils which leads to the key inflammatory response resulting in blisters. Importantly, the inhibitory FcγRIIB receptor is required for protection (Kasperkiewicz, Nimmerjahn et al. 2012). The Fc^{F241A} therapeutic mechanism of action is thought to involve upregulation of FcγRIIB on B cells and myeloid cells (including neutrophils, the key mediator of disease in this model). We therefore assessed treatment-mediated changes in FcγRIIB cell surface expression on circulating B cells and myeloid cells. As shown in FIG. 11B, Fc^{F241A} uniquely resulted in a statistically significant increase in circulating peripheral blood neutrophil surface expression of FcγRIIB, relative to PBS control. Efgartigimod did not induce any changes in FcγRIIB expression.

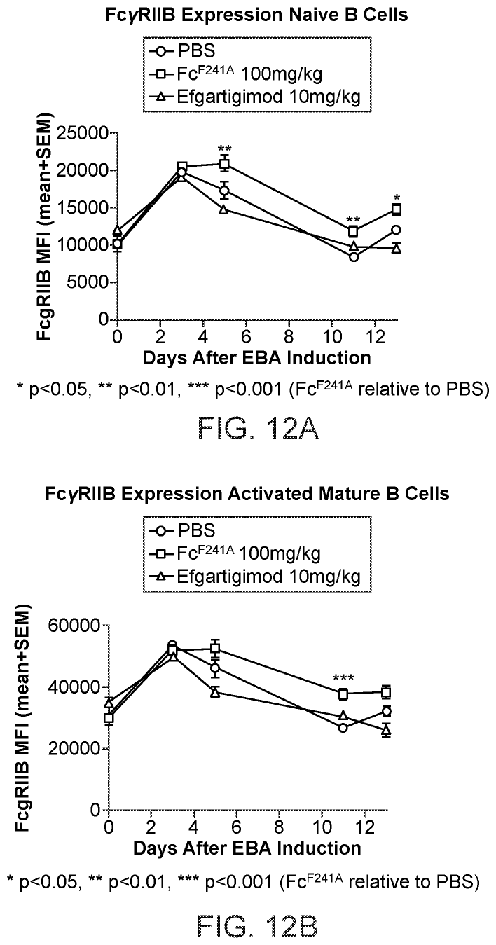

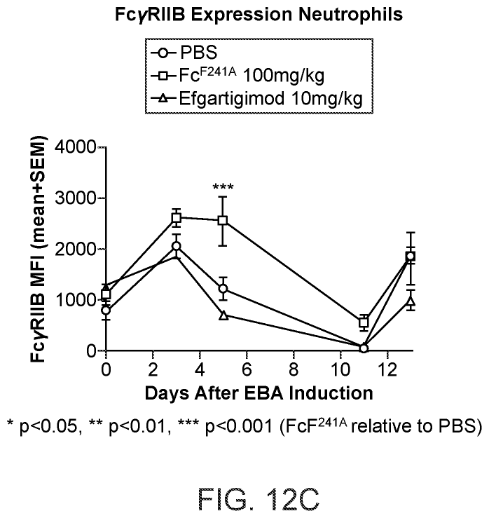

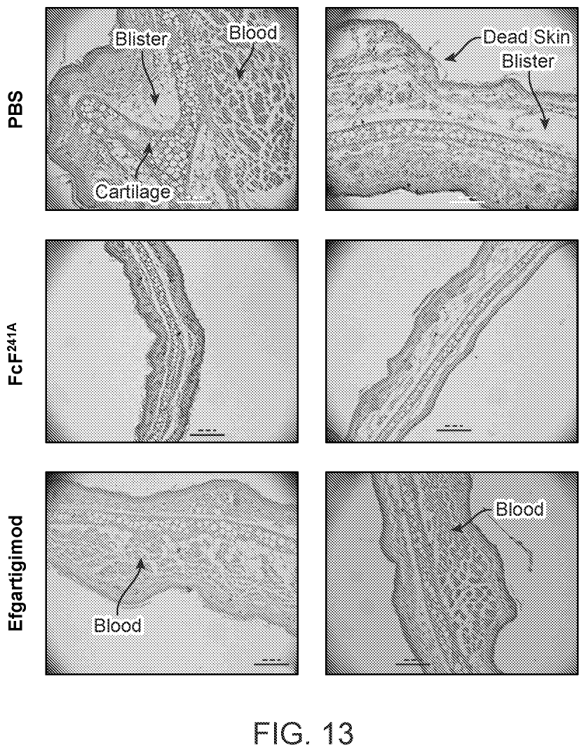

The kinetics of Fc^{F241A}-induced FcγRIIB cell surface expression by all responding leukocyte populations in blood are shown in FIGS. 12A-12C. These included naïve B cells (FIG. 12A), activated mature B cells (FIG. 12B), and neutrophils (FIG. 12C). The data suggest that Fc^{F241A} has the potential to maintain elevated FcγRIIB cell surface expression for 7-9 days following the last dose (on study day 3).

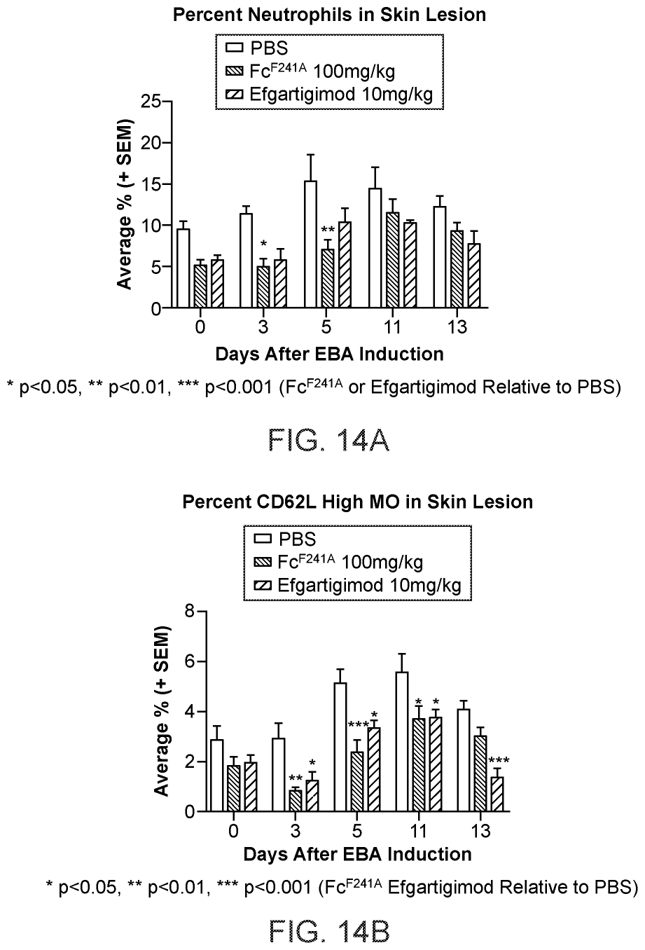

Biopsies from disease-affected ear skin were collected from the mice at study termination (day 13) and H&E staining was performed, with images presented in FIG. 13. Two representative images from the PBS control (top row), Fc^{F241A} (middle row) and efgartigimod (bottom row) treatment groups are shown. The images are annotated to highlight cartilage, and areas affected by blisters, pooling of blood, and dead skin. The treatment effect by Fc^{F241A} leads to near complete protection from skin damage resulting from the pathogenic autoantibody. Importantly, both Fc^{F241A} and efgartigimod led to an early decrease in the infiltration of neutrophils to the skin tissue, with Fc^{F241A} resulting in significant reductions relative to PBS control on study days 3-5 (FIG. 14A). Both Fc^{F241A} and efgartigimod resulted in significant decreases in infiltrating CD62 ligand positive monocytes in the skin tissue on days 3-13 (FIG. 14B).

Fc^{F241A} is efficacious in a mouse model of pemphigoid disease and is associated with increased FcγRIIB surface expression on circulating neutrophils and decreased skin infiltration of neutrophils, the disease-causing immune cell in this model. These data suggest that Fc^{F241A} mediated upregulation of the immune checkpoint receptor FcγRIIB may be therapeutically meaningful in patients with pemphigoid diseases, including bullous pemphigoid (BP) and epidermolysis bullosa acquisita (EBA).

### Example 10: Fc^{F241A} is Protective in the MOG 35-55 Induced EAE Model of T Cell-Mediated Neuroinflammation in Mice

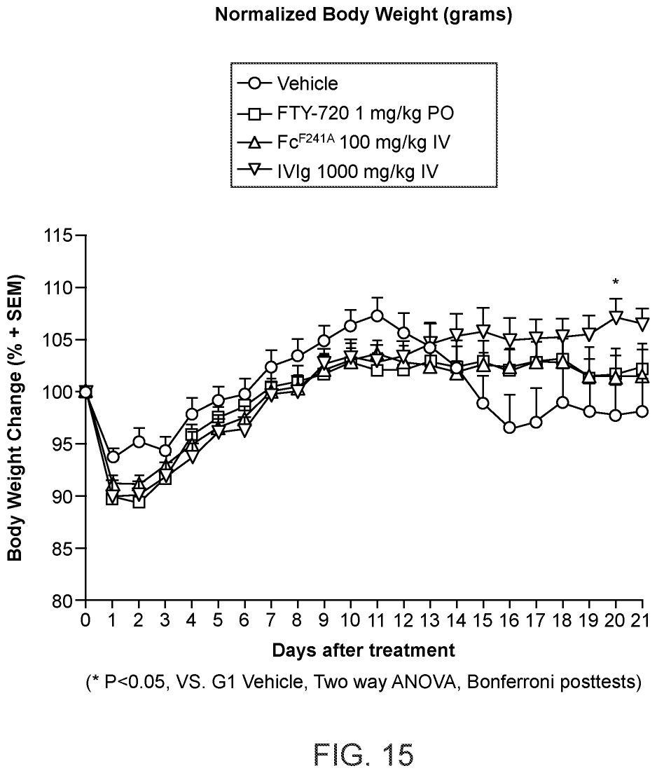

Experimental autoimmune encephalomyelitis (EAE) was induced in mice, followed by randomization into treatment groups (n=7 per group). Body weights were recorded daily, beginning study day 0 through day 21, and the data were normalized to their baseline weight on study day 0. As shown in FIG. 15, all treatment groups demonstrated an initial drop in body weight (~10%) in the first few days following EAE induction, followed by a recovery. As disease developed, the mean body weight of vehicle treated mice decreased to a greater degree than any of the therapeutic intervention groups. This was largely non statistically significant, however. Overall, the results suggest that Fc^{F241A} exhibits a non-significant trend in protection from body weight loss.

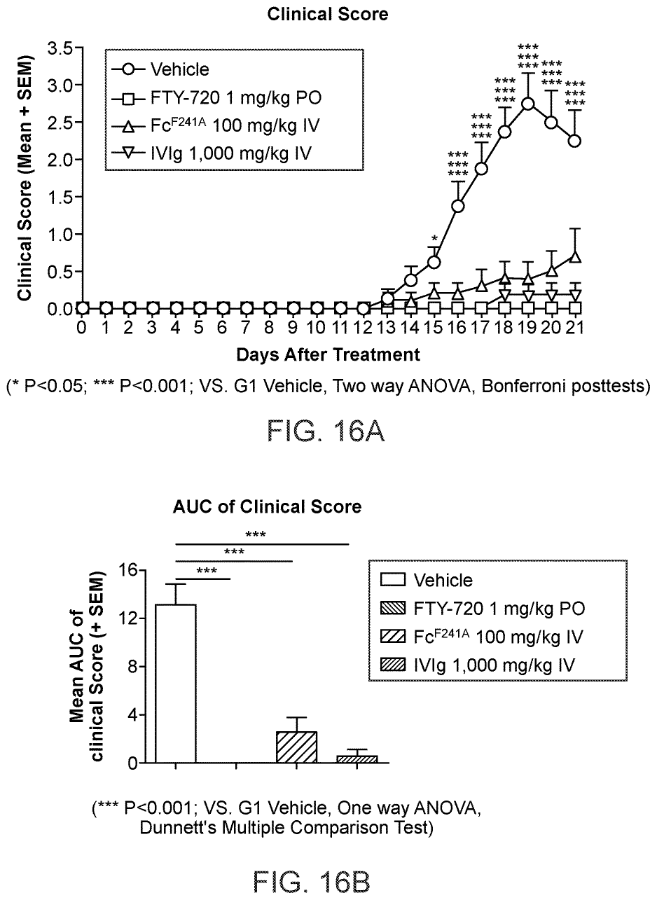

Clinical inflammation scores are presented in FIGS. 16A and 16B. As shown in FIG. 16A, the onset of measurable disease occurred on study day 13 for the vehicle control group and rapidly escalated until plateauing on study day 19. Significant protection over the entire course of the study was observed for FTY-720, high-dose IVIg, and Fc^{F241A} treated mice. The area under the curve (AUC) for the entire time-course of inflammation scores for each group are shown separately in the FIG. 16B. All three therapeutic interventions demonstrated significant protection (p<0.001) relative to vehicle control. Among these three therapeutic intervention groups, there was no significant difference in protection at any time point.

These data confirm the protective activity of Fc^{F241A} against T cell-mediated neuroinflammation in the EAE model and demonstrate a mechanism that involves the control of effector T cell-mediated inflammation. Without wishing to be bound by any particular theory, the protective effects of IVIg and Fc^{F241A} may be a function of their ability to expand Tregs, as previously reported for the sialylated IVIg and Fc-F241A mechanism (Fiebiger B M, et al. “Protection in antibody- and T cell-mediated autoimmune diseases by antiinflammatory IgG Fcs requires type II FcRs.” Proc Natl Acad Sci USA. 2015 May 5; 112(18):E2385-94.)

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure. Other embodiments are in the claims.

1. A population of modified Fc polypeptides, each modified Fc polypeptide having (i) an amino acid sequence at least 75% identical to the sequence of SEQ ID NO: 2 and (ii) an aliphatic amino acid residue at position 241 (numbered according to Kabat; corresponding to amino acid residue 32 of SEQ ID NO: 2), comprising: at least 60% of the modified Fc polypeptides having a sialic acid (SA) moiety attached to an N-glycan of the Fc polypeptide via an α(2,6) linkage.

2. The population of claim 1, wherein the aliphatic amino acid at position 241 is an alanine (Ala; F241A).

3. The population of claim 1 or 2, wherein the N-glycan is attached to the asparagine (Asn) at amino acid residue 297 of the polypeptide (Asn297; numbered according to Kabat; corresponding to amino acid residue 88 of SEQ ID NO: 2).

4. The population of any one of claims 1-3, wherein at least 70% of the Fc polypeptides comprise the SA moiety attached to the N-glycan of the Fc polypeptides via the α(2,6) linkage.

5. The population of any one of claims 1-4, wherein at least 80% of the Fc polypeptides comprise the SA moiety attached to the N-glycan of the Fc polypeptides via the α(2,6) linkage.

6. The population of any one of claims 1-5, wherein at least 90% of the Fc polypeptides comprise the SA moiety attached to the N-glycan of the Fc polypeptides via the α(2,6) linkage.

7. The population of any one of claims 1-6, wherein the N-glycan of the Fc polypeptides is mono-sialylated or di-sialylated.

8. The population of claim 7, wherein at least 30% of the Fc polypeptides comprise mono-sialylated N-glycans comprising a SA moiety attached via the α(2,6) linkage.

9. The population of claim 7, wherein at least 30% of the Fc polypeptides comprise di-sialylated N-glycans comprising two SA moieties attached via the α(2,6) linkage.

10. The population of claim 9, wherein about 90% of the Fc polypeptides comprise di-sialylated N-glycans comprising two SA moieties attached via the α(2,6) linkage.

11. The population of any one of claims 1-10, wherein at least about 60% of the Fc polypeptides comprise a galactose moiety.

12. The population of any one of claims 1-11, wherein at least about 70% of the Fc polypeptides comprise a galactose moiety.

13. The population of any one of claims 1-12, wherein at least about 80% of the Fc polypeptides comprise a galactose moiety.

14. The population of any one of claims 1-13, wherein at least about 90% of the Fc polypeptides comprise a galactose moiety.

15. The population of any one of claims 1-14, wherein about 100% of the Fc polypeptides comprise a galactose moiety.

16. The population of any one of claims 11-15, wherein the galactose moiety is attached to an α(1,3) arm and/or α(1,6) arm of the N-glycan.

17. The population of any one of claims 11-16, wherein the galactose moiety is a branched galactose moiety.

18. A population of modified Fc polypeptides, each modified Fc polypeptide having (i) an amino acid sequence at least 75% identical to the sequence of SEQ ID NO: 2 and (ii) an aliphatic amino acid residue at position 241 (numbered according to Kabat; corresponding to amino acid residue 32 of SEQ ID NO: 2), comprising: about 40% of the modified Fc polypeptides having a SA moiety attached to the N-glycan of the Fc polypeptide via an α(2,3) linkage.

19. The population of claim 16, wherein the aliphatic amino acid at position 241 is an Ala (F241A).

20. The population of any one of claims 1-19, wherein the Fc polypeptides are IgG1.

21. The population of any one of claims 1-19, wherein the Fc polypeptides are IgG3.

22. A pharmaceutical composition, comprising: (a) the population of any one of claims 1-21 and (b) a pharmaceutically acceptable carrier, diluent, or excipient.

23. The pharmaceutical composition of claim 22, wherein the pharmaceutically acceptable carrier, diluent, or excipient is selected from the group consisting of: a stabilizer, buffer, surfactant, filler, solvent, tonicity or osmolarity adjusting agent, antioxidant, adjuvant, and antimicrobial agent.

24. A method of treating an inflammatory disease or condition in a subject in need thereof, comprising: administering to the subject a therapeutically effective amount of the population of any one of claims 1-21 or the pharmaceutical composition of claim 22 or 23.

25. The method of claim 24, wherein the inflammatory disease or condition is an autoimmune disease or condition.

26. The method of claim 24 or 25, wherein the inflammatory disease or condition is arthritis.

27. The method of claim 24 or 25, wherein the inflammatory disease or condition is immune thrombocytopenia (ITP).

28. The method of any one of claims 24-27, wherein the population or the pharmaceutical composition has a half-life of at least 3.5 days following administration of the population or the pharmaceutical composition to the subject.

29. The method of any one of claims 24-28, wherein the population or the pharmaceutical composition has a half-life of at least 4 days following administration of the population or the pharmaceutical composition to the subject.

30. The method of any one of claims 24-29, wherein the population or the pharmaceutical composition is cleared from the circulation of the subject at a rate of no greater than 45 mL/day/kg.

31. The method of any one of claims 24-30, wherein the population or the pharmaceutical composition is cleared from the circulation of the subject at a rate of no greater than 40 mL/day/kg.

32. The method of any one of claims 24-31, wherein the population or the pharmaceutical composition is cleared from the circulation of the subject at a rate of no greater than 35 mL/day/kg.

33. The method of any one of claims 24-32, wherein the population or the pharmaceutical composition is cleared from the circulation of the subject at a rate of no greater than 30 mL/day/kg.

34. The method of any one of claims 24-33, wherein the population or the pharmaceutical composition is cleared from the circulation of the subject at a rate of no greater than 25 mL/day/kg.

35. The method of any one of claims 24-34, wherein the concentration of the Fc polypeptide over time (AUC) is at least 480 day×mg/mL between 1 and 35 days following administration of the population or the pharmaceutical composition to the subject.

36. The method of claim 35, wherein the AUC is at least 500 day×mg/mL, at least 550 day×mg/mL, at least 600 day×mg/mL, at least 650 day×mg/mL, at least 700 day×mg/mL, at least 750 day×mg/mL, at least 800 day×mg/mL, or at least 850 day×mg/mL between 1 and 35 days following administration of the population or the pharmaceutical composition to the subject.

37. The method of any one of claims 24-36, further comprising administration of an additional therapeutic agent to the subject.

38. The method of claim 37, wherein the additional therapeutic agent is administered to the subject prior to, concurrently with, or subsequent to administration of the population or the pharmaceutical composition.

39. The method of claim 37 or 38, wherein the additional therapeutic agent is a second modified Fc polypeptide comprising one or more amino acid substitutions selected from the group consisting of M252Y, S254T, T256E, H433K, and N434F.

40. The method of claim 39, wherein the second modified Fc polypeptide comprises amino acid substitutions M252Y, S254T, T256E, H433K, and N434F.

41. The method of claim 39 or 40, wherein the second modified Fc polypeptide has an amino acid sequence set forth as SEQ ID NO: 5.

42. The method of any one of claims 39-41, wherein the second modified Fc polypeptide is administered at a dose of 1 mg/kg to 20 mg/kg.

43. The method of any one of claims 39-42, wherein the second modified Fc polypeptide is administered at a dose of 10 mg/kg.

44. The method of any one of claims 39-43, wherein the second modified Fc polypeptide is administered once weekly for four weeks.

45. The method of any one of claims 37-44, wherein the additional therapeutic agent is selected from the group consisting of an anti-inflammatory agent, an immune-suppressive agent, an analgesic, a disease-modifying antirheumatic drug (DMARD), a counterirritant, a platelet-boosting drug, a thrombopoietin receptor (TPOR) agonist, physical therapy, and surgery.

46. The method of claim 45, wherein the anti-inflammatory agent is selected from the group consisting of non-steroidal anti-inflammatory drug (NSAID), corticosteroid, anti-inflammatory antibody or an antigen-binding fragment thereof, anti-inflammatory cytokine, kinase inhibitor, and intravenous immunoglobulin (IVIG).

47. A nucleic acid expression vector, comprising:
(a) a first expression cassette, comprising: a first mammalian promoter operably linked to a polynucleotide encoding a modified Fc polypeptide having: (i) an amino acid sequence at least 75% identical to the sequence of SEQ ID NO: 2 and (ii) an aliphatic amino acid residue at position 241 (numbered according to Kabat; corresponding to amino acid residue 32 of SEQ ID NO: 2); and
(b) a second expression cassette, comprising: a second mammalian promoter operably linked to a polynucleotide encoding a beta-galactoside alpha-2,6-sialyltransferase 1 (ST6GAL1) enzyme.

48. The expression vector of claim 47, wherein the aliphatic amino acid at position 241 is an Ala (F241A).

49. The expression vector of claim 48, wherein the second expression cassette further comprises a polynucleotide encoding a beta-1,4-galactosyltransferase 1 (B4GALT1) enzyme.

50. The expression vector of claim 49, wherein the polynucleotide encoding the B4GALT1 enzyme is operatively linked to the second promoter.

51. The expression vector of any one of claims 47-50, wherein the second expression cassette further comprises an internal ribosome entry site (TRES) sequence positioned between the polynucleotide encoding the ST6GAL1 enzyme and the polynucleotide encoding the B4GALT1 enzyme.

52. The expression vector of any one of claims 47-51, wherein the first promoter and the second promoter are each independently selected from the group consisting of a murine cytomegalovirus (CMV) promoter, elongation factor 1α (EF1α) promoter, eukaryotic elongation factor 2 (EEF2) promoter, glyceraldehyde 3-phosphate dehydrogenase (GAPDH) promoter, phosphoglycerate kinase (PGK) promoter, actin promoter, and ubiquitin promoter.

53. A mammalian host cell, comprising: the expression vector according to any one of claims 47-52.

54. The mammalian host cell of claim 53, wherein the mammalian host cell is a Chinese hamster ovary (CHO) cell.

55. The mammalian host cell of claim 53, wherein the mammalian host cell is a human embryonic kidney 293 (HEK293) cell.

56. A method of manufacturing a population of modified Fc polypeptides, each modified Fc polypeptide having: (i) an amino acid sequence having at least 75% identity to SEQ ID NO: 2, and (ii) an aliphatic amino acid residue at position 241 (numbered according to Kabat; corresponding to amino acid residue 32 of SEQ ID NO: 2),
the method comprising: (a) culturing a mammalian host cell of any one of claims 40-42 under conditions and for a time that induces expression of the first expression cassette and the second expression cassette, thus producing the population of modified Fc polypeptides; and (b) purifying the population of modified Fc polypeptides.

57. The method of claim 56, wherein the aliphatic amino acid at position 241 is an Ala (F241A).

58. The method of claim 56 or 57, wherein at least 60% of the modified Fc polypeptides have a SA moiety attached to an N-glycan of the Fc polypeptide via an α(2,6) linkage.

59. The method of any one of claims 56-58, wherein at least 70% of the Fc polypeptides comprise the SA moiety attached to the N-glycan of the Fc polypeptides via the α(2,6) linkage.

60. The method of any one of claims 56-59, wherein at least 80% of the Fc polypeptides comprise the SA moiety attached to the N-glycan of the Fc polypeptides via the α(2,6) linkage.

61. The method of any one of claims 56-60, wherein at least 90% of the polypeptides comprise a SA moiety attached to the N-glycan via the α(2,6) linkage.

62. The method of any one of claims 56-61, wherein the first promoter and the second promoter are each independently selected from the group consisting of a murine CMV promoter, EF1α promoter, EEF2 promoter, GAPDH promoter, PGK promoter, actin promoter, and ubiquitin promoter.

63. The method of any one of claims 56-62, wherein the first expression cassette and/or the second expression cassette each independently comprise one or more regulatory sequences selected from the group consisting of a 5′ untranslated region (UTR), 3′ UTR, enhancer, insulator, intron, RNA export element, polyadenylation signal, internal ribosome entry site (IRES), and transcription terminator.

64. The method of any one of claims 56-63, wherein the N-glycan of the Fc polypeptides is mono-sialylated or di-sialylated.

65. The method of claim 64, wherein about 30% of the Fc polypeptides comprise mono-sialylated N-glycans comprising a SA moiety attached via the α(2,6) linkage.

66. The method of claim 64, wherein about 30% of the Fc polypeptides comprise di-sialylated N-glycans comprising two SA moieties attached via the α(2,6) linkage.

67. The method of claim 66, wherein about 90% of the Fc polypeptides comprise di-sialylated N-glycans comprising two SA moieties attached via the α(2,6) linkage.

68. The method of any one of claims 56-67, wherein at least about 60% of the Fc polypeptides comprise a galactose moiety.

69. The method of any one of claims 56-68, wherein at least about 70% of the Fc polypeptides comprise a galactose moiety.

70. The method of any one of claims 56-69, wherein at least about 80% of the Fc polypeptides comprise a galactose moiety.

71. The method of any one of claims 56-70, wherein at least about 90% of the Fc polypeptides comprise a galactose moiety.

72. The method of any one of claims 56-71, wherein about 100% of the Fc polypeptides comprise a galactose moiety.

73. The method of any one of claims 56-72, wherein the galactose moiety is attached to an α(1,3) arm and/or α(1,6) arm of the N-glycan.

74. The method of any one of claims 56-73, wherein the galactose moiety is a branched galactose moiety.

75. The method of any one of claims 56-74, wherein the mammalian host cell comprises ST6GAL1 and B4GALT1 at a ratio of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20 (mol:mol).

76. The method of any one of claims 56-75, wherein the mammalian host cell is in a population of mammalian host cells in which at least 50%, at least 60%, at least 70%, at least 80%, at least 90%, or more of the mammalian host cells remain viable after between 10 and 20 days following the start of step (a).

77. The method of any one of claims 56-76, further comprising contacting the mammalian host cell with an additive that enhances sialylation of the Fc polypeptides.

78. The method of claim 77, wherein the additive is selected from the group consisting of uridine, manganese, copper, dexamethasone, hydrocortisone, N-acetyl mannosamine, tetraacetylated ManNAc, N-azidoacetyl D-mannosamine, 1,3,4-O-Bu₃ManNAc, α(2,3)-Dehydro-2-deoxy-N-acetylneuraminic acid (DANA), siastatin B, fetuin, and glycerol.

79. The method of any one of claims 56-78, wherein glycosylation of the N-glycan is determined using HPLC, MS, or a combination thereof.

80. The method of claim 79, wherein the HPLC is hydrophilic interaction liquid chromatography (HILIC).

81. The population of any one of claims 1-21 or the pharmaceutical composition of claim 22 or 23 for use as a medicament.

82. The population of any one of claims 1-21 or the pharmaceutical composition of claim 22 or 23 for use in the treatment of an inflammatory disease or condition in a subject in need thereof.

83. The population or pharmaceutical composition of claim 82, wherein the inflammatory disease or condition is an autoimmune disease or condition.

84. The population or pharmaceutical composition of claim 82 or 83, wherein the inflammatory disease or condition is arthritis.

85. The population or pharmaceutical composition of claim 82 or 83, wherein the inflammatory disease or condition is immune thrombocytopenia (ITP).

86. The population or pharmaceutical composition of any one of claims 81-85, wherein the population or the pharmaceutical composition has a half-life of at least 3.5 days following administration of the population or the pharmaceutical composition to a subject.

87. The population or pharmaceutical composition of claim 86, wherein the population or the pharmaceutical composition has a half-life of at least 4 days following administration of the population or the pharmaceutical composition to a subject.

88. The population or pharmaceutical composition of any one of claims 81-87, wherein the population or the pharmaceutical composition, following administration to a subject, is cleared from the circulation of the subject at a rate of no greater than 45 mL/day/kg.

89. The population or pharmaceutical composition of claim 88, wherein the population or the pharmaceutical composition is cleared from the circulation of the subject at a rate of no greater than 40 mL/day/kg.

90. The population or pharmaceutical composition of claim 89, wherein the population or the pharmaceutical composition is cleared from the circulation of the subject at a rate of no greater than 35 mL/day/kg.

91. The population or pharmaceutical composition of claim 90, wherein the population or the pharmaceutical composition is cleared from the circulation of the subject at a rate of no greater than 30 mL/day/kg.

92. The population or pharmaceutical composition of claim 91, wherein the population or the pharmaceutical composition is cleared from the circulation of the subject at a rate of no greater than 25 mL/day/kg.

93. The population or pharmaceutical composition of any one of claims 81-92, wherein the concentration of the Fc polypeptide over time (AUC) is at least 480 day×mg/mL between 1 and 35 days following administration of the population or the pharmaceutical composition to a subject.

94. The population or pharmaceutical composition of claim 93, wherein the AUC is at least 500 day×mg/mL, at least 550 day×mg/mL, at least 600 day×mg/mL, at least 650 day×mg/mL, at least 700 day×mg/mL, at least 750 day×mg/mL, at least 800 day×mg/mL, or at least 850 day×mg/mL between 1 and 35 days following administration of the population or the pharmaceutical composition to a subject.

95. The population or pharmaceutical composition of any one of claims 81-94, wherein an additional therapeutic agent is administered to the subject.

96. The population or pharmaceutical composition of claim 95, wherein the additional therapeutic agent is administered to the subject prior to, concurrently with, or subsequent to administration of the population or the pharmaceutical composition.

97. The population or pharmaceutical composition of claim 95 or 96, wherein the additional therapeutic agent is a second modified Fc polypeptide comprising one or more amino acid substitutions selected from the group consisting of M252Y, S254T, T256E, H433K, and N434F.

98. The population or pharmaceutical composition of claim 97, wherein the second modified Fc polypeptide comprises amino acid substitutions M252Y, S254T, T256E, H433K, and N434F.

99. The population or pharmaceutical composition of claim 98, wherein the second modified Fc polypeptide has an amino acid sequence set forth as SEQ ID NO: 5.

100. The population or pharmaceutical composition of any one of claims 97-99, wherein the second modified Fc polypeptide is administered to a subject at a dose of 1 mg/kg to 20 mg/kg.

101. The population or pharmaceutical composition of claim 100, wherein the second modified Fc polypeptide is administered to the subject at a dose of 10 mg/kg.

102. The population or pharmaceutical composition of any one of claims 97-101, wherein the second modified Fc polypeptide is administered to a subject once weekly for four weeks.

103. The population or pharmaceutical composition of any one of claims 95-102, wherein the additional therapeutic agent is selected from the group consisting of an anti-inflammatory agent, an immune-suppressive agent, an analgesic, a disease-modifying antirheumatic drug (DMARD), a counterirritant, a platelet-boosting drug, a thrombopoietin receptor (TPOR) agonist, physical therapy, and surgery.

104. The population or pharmaceutical composition of claim 103, wherein the anti-inflammatory agent is selected from the group consisting of non-steroidal anti-inflammatory drug (NSAID), corticosteroid, anti-inflammatory antibody or an antigen-binding fragment thereof, anti-inflammatory cytokine, kinase inhibitor, and intravenous immunoglobulin (IVIG).


SEQUENCE LISTING (6 sequences)

SEQ ID NO: 1 - protein, 238 aa, Homo sapiens
KVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 238 aa, synthetic construct
KVDKKVEPKSCDKTHTCPPCPAPELLGGPSVALFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 4 - protein, 398 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 5 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 6 - protein, 238 aa, synthetic construct
KVDKRVEPKSCDKTHTCPPCPAPELLGGPSVALFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK